(19) United States
(12) Patent Application Publication
(10) Pub. No.: US 2023/0212267 A1
(43) Pub. Date: Jul. 6, 2023

(54) DNA ANTIBODY CONSTRUCTS FOR USE AGAINST SARS-COV-2

(71) Applicants: David WEINER, Merion, PA (US); Kar MUTHUMANI, Cherry Hill, NJ (US); Elizabeth PARZYCH, Philadelphia, PA (US); The Wistar Institute of Anatomy and Biology, Philadelphia, PA (US)

(72) Inventors: David Weiner, Merion, PA (US); Elizabeth Parzych, Philadelphia, PA (US); Kar Muthumani, Cherry Hill, NJ (US)

(21) Appl. No.: 18/000,886

(22) PCT Filed: Jun. 9, 2021
(86) PCT No.: PCT/US2021/036606

§ 371 Date: Dec. 6, 2022

Related U.S. Application Data
(60) Provisional application No. 63/036,795, filed on Jun. 9, 2020.
(60) Provisional application No. 63/036,809, filed on Jun. 9, 2020.
(60) Provisional application No. 63/068,868, filed on Aug. 21, 2020.
(60) Provisional application No. 63/083,173, filed on Sep. 25, 2020.
(60) Provisional application No. 63/114,271, filed on Nov. 16, 2020.

(51) Int. Cl. C07K 16/10; C12N 15/63; A61P 31/14

(52) U.S. Cl. CPC C07K 16/10; C12N 15/63; A61P 31/14; C07K 2317/565

(57) ABSTRACT

Disclosed herein are antibodies to SARS-CoV-2 antigens and recombinant nucleic acid sequences that encode the SARS-CoV-2 antibodies. The disclosure also provides a method of preventing and/or treating a SARS-CoV-2 infection or disease or disorder associated with SARS-CoV-2 infection (e.g., COVID-19) in a subject using said compositions and methods.

### CROSS REFERENCE TO RELATED APPLICATIONS

This application claims priority to U.S. Provisional Application No. 63/036,809, filed Jun. 9, 2020; U.S. Provisional Application No. 63/036,795, filed Jun. 9, 2020; U.S. Provisional Application No. 63/068,868, filed Aug. 21, 2020; U.S. Provisional Application No. 63/083,173, filed Sep. 25, 2020; and U.S. Provisional Application No. 63/114,271, filed Nov. 16, 2020, each of which is hereby incorporated by reference herein in its entirety.

### TECHNICAL FIELD

The present invention relates to a composition comprising a recombinant nucleic acid sequence for generating one or more synthetic antibodies, and functional fragments thereof, in vivo, and a method of preventing and/or treating viral infection in a subject by administering said composition.

### BACKGROUND

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, OC43, NL63, and HKU1 are endemic in the human population.

COVID-19, known previously as 2019-nCoV pneumonia or disease, has rapidly emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. In December 2019 the city of Wuhan in China became the epicenter for an outbreak of the novel coronavirus, SARS-CoV-2. SARS-CoV-2 was isolated and sequenced from human airway epithelial cells from infected patients (Zhu et al., 2020 N Engl J Med, 382:727-733; Wu et al., 2020, Nature, 579:265-269). Disease symptoms can range from mild flu-like to severe cases with life-threatening pneumonia (Huang et al., 2020, Lancet, 395:497-506). The global situation is dynamically evolving, and on Jan. 30, 2020 the World Health Organization declared COVID-19 as a public health emergency of international concern (PHEIC) and on Mar. 11, 2020 it was declared a global pandemic. As of Apr. 1, 2020 there were 932,605 people infected and 46,809 deaths (gisaid.org/epiflu-applications/global-cases-covid-19). Infections have spread to multiple continents. Human-to-human transmission has been observed in multiple countries, and a shortage of disposal personal protective equipment, and prolonged survival times of coronaviruses on inanimate surfaces (Hulkower et al., 2011, Am J Infect Control 39, 401-407), have compounded this already delicate situation and heightened the risk of nosocomial infections. Advanced research activities must be pursued in parallel to push forward protective modalities in an effort to protect billions of vulnerable individuals worldwide.

Thus, there is need in the art for therapeutics that prevent and/or treat COVID-19, thereby providing protection against and promoting survival of COVID-19 infection. The current invention satisfies this need.

### SUMMARY OF THE INVENTION

In one embodiment, the invention relates to an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody.

In one embodiment, the immunoconjugate comprises a therapeutic agent or a detection moiety.

In one embodiment, the antibody is selected from the group consisting of a humanized antibody, a chimeric antibody, a fully human antibody, an antibody mimetic.

In one embodiment, the antibody comprises at least one selected from the group consisting of: a) the heavy chain CDR1 sequence selected from the group consisting of SEQ ID NO:26, SEQ ID NO: 80 and SEQ ID NO:108; b) the heavy chain CDR2 sequence selected from the group consisting of SEQ ID NO:27, SEQ ID NO: 81 and SEQ ID NO:109; c) the heavy chain CDR3 sequence selected from the group consisting of SEQ ID NO:28, SEQ ID NO: 82 and SEQ ID NO:110; d) the light chain CDR1 sequence selected from the group consisting of SEQ ID NO:34, SEQ ID NO: 88 and SEQ ID NO:116; e) the light chain CDR2 sequence selected from the group consisting of SEQ ID NO:35, SEQ ID NO: 89 and SEQ ID NO:117; and f) the light chain CDR3 sequence selected from the group consisting of SEQ ID NO:36, SEQ ID NO: 90 and SEQ ID NO:118.

In one embodiment, the antibody comprises at least one selected from the group consisting of: a) an anti-SARS-CoV-2 antibody heavy chain comprising a sequence having at least 80% identity to an amino acid sequence selected from the group consisting of SEQ ID NO:2, SEQ ID NO:6, SEQ ID NO: 12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; b) an anti-SARS-CoV-2 antibody light chain comprising a sequence having at least 80% identity to an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130; c) a fragment of an anti-SARS-CoV-2 antibody heavy chain comprising at least 80% of the full length sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and d) a fragment of an anti-SARS-CoV-2 antibody light chain comprising at least 80% of the full length sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130.

In one embodiment, the antibody comprises at least one selected from the group consisting of: a) an amino acid sequence having at least 80% identity to an amino acid sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132; and b) a fragment of an anti-SARS-CoV-2 antibody heavy chain comprising at least 80% of the full length sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132.

In one embodiment, the invention relates to a nucleic acid molecule encoding an anti-SARS-CoV-2 antibody or fragment thereof wherein the antibody is selected from the group consisting of a monoclonal antibody, a polyclonal antibody, a single chain antibody, an immunoconjugate, a defucosylated antibody, and a bispecific antibody. In one embodiment, the immunoconjugate comprises a therapeutic agent or a detection moiety.

In one embodiment, the nucleic acid molecule encodes an antibody selected from the group consisting of a humanized antibody, a chimeric antibody, a fully human antibody, an antibody mimetic.

In one embodiment, the nucleic acid molecule encodes an antibody comprising at least one selected from the group consisting of: a) the heavy chain CDR1 sequence selected from the group consisting of SEQ ID NO:26, SEQ ID NO: 80 and SEQ ID NO:108; b) the heavy chain CDR2 sequence selected from the group consisting of SEQ ID NO:27, SEQ ID NO: 81 and SEQ ID NO:109; c) the heavy chain CDR3 sequence selected from the group consisting of SEQ ID NO:28, SEQ ID NO: 82 and SEQ ID NO:110; d) the light chain CDR1 sequence selected from the group consisting of SEQ ID NO:34, SEQ ID NO: 88 and SEQ ID NO:116; e) the light chain CDR2 sequence selected from the group consisting of SEQ ID NO:35, SEQ ID NO: 89 and SEQ ID NO:117; and f) the light chain CDR3 sequence selected from the group consisting of SEQ ID NO:36, SEQ ID NO: 90 and SEQ ID NO:118.

In one embodiment, the nucleic acid molecule encodes an antibody comprising at least one selected from the group consisting of: a) an anti-SARS-CoV-2 antibody heavy chain comprising a sequence having at least 80% identity to an amino acid sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; b) an anti-SARS-CoV-2 antibody light chain comprising a sequence having at least 80% identity to an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130; c) a fragment of an anti-SARS-CoV-2 antibody heavy chain comprising at least 80% of the full length sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and d) a fragment of an anti-SARS-CoV-2 antibody light chain comprising at least 80% of the full length sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130.

In one embodiment, the nucleic acid molecule encodes an antibody comprising at least one selected from the group consisting of: a) an amino acid sequence having at least 80% identity to an amino acid sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132; and b) a fragment of an anti-SARS-CoV-2 antibody heavy chain comprising at least 80% of the full length sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132.

In one embodiment, the nucleic acid molecule further comprises a nucleotide sequence encoding a cleavage domain.

In one embodiment, the nucleic acid molecule comprises at least one selected from the group consisting of: a) a nucleotide sequence selected from the group consisting of SEQ ID NO:23, SEQ ID NO: 77 and SEQ ID NO:105 encoding the heavy chain CDR1 sequence; b) a nucleotide sequence selected from the group consisting of SEQ ID NO:24, SEQ ID NO: 78 and SEQ ID NO:106 encoding the heavy chain CDR2 sequence; c) a nucleotide sequence selected from the group consisting of SEQ ID NO:25, SEQ ID NO: 79 and SEQ ID NO:107 encoding the heavy chain CDR3 sequence; d) a nucleotide sequence selected from the group consisting of SEQ ID NO:31, SEQ ID NO: 85 and SEQ ID NO:113 encoding the light chain CDR1 sequence; e) a nucleotide sequence selected from the group consisting of SEQ ID NO:32, SEQ ID NO: 86 and SEQ ID NO:114 encoding the light chain CDR2 sequence; and f) a nucleotide sequence selected from the group consisting of SEQ ID NO:33, SEQ ID NO: 87 and SEQ ID NO:115 encoding the light chain CDR3 sequence.

In one embodiment, the nucleic acid molecule comprises at least one nucleotide sequence selected from the group consisting of: a) a nucleotide sequence having at least 80% identity to a nucleotide selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; b) a nucleotide sequence having at least 80% identity to a nucleotide selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain; c) a fragment of a nucleotide sequence comprising at least 80% of the full length sequence selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and d) a fragment of a nucleotide sequence comprising at least 80% of the full length sequence selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain.

In one embodiment, the nucleic acid molecule comprises a nucleotide sequence selected from the group consisting of: a) a nucleotide sequence having at least 80% identity to a nucleotide selected from the group consisting of SEQ ID NO:9, SEQ ID NO:15, SEQ ID NO:21, SEQ ID NO:39, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:97, SEQ ID NO:103, SEQ ID NO:125 and SEQ ID NO:131; and b) a fragment of a nucleotide sequence comprising at least 80% of the full length sequence selected from the group consisting of SEQ ID NO:9, SEQ ID NO:15, SEQ ID NO:21, SEQ ID NO:39, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:97, SEQ ID NO:103, SEQ ID NO:125 and SEQ ID NO:131.

In one embodiment, the nucleotide sequence encodes a leader sequence.

In one embodiment, the nucleic acid molecule comprises an expression vector.

In one embodiment, the invention relates to a composition comprising anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody.

In one embodiment, the invention relates to a composition comprising at least one nucleic acid molecule encoding an anti-SARS-CoV-2 antibody or fragment thereof wherein the antibody is selected from the group consisting of a monoclonal antibody, a polyclonal antibody, a single chain antibody, an immunoconjugate, a defucosylated antibody, and a bispecific antibody. In one embodiment, the immunoconjugate comprises a therapeutic agent or a detection moiety.

In one embodiment, the composition comprises a first nucleic acid molecule comprising a nucleotide sequence encoding a heavy chain of an anti-SARS-CoV-2 spike antigen synthetic antibody; and a second nucleic acid molecule comprising a nucleotide sequence encoding a light chain of an anti-SARS-CoV-2 spike antigen synthetic antibody.

In one embodiment, the first nucleic acid molecule comprises a nucleotide sequence encoding at least one selected from the group consisting of: a) an anti-SARS-CoV-2 antibody heavy chain comprising a sequence having at least 80% identity to an amino acid sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and b) a fragment of an anti-SARS-CoV-2 antibody heavy chain comprising at least 80% of the full length sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and the second nucleic acid molecule comprises a nucleotide sequence encoding at least one selected from the group consisting of: c) an anti-SARS-CoV-2 antibody light chain comprising a sequence having at least 80% identity to an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130; and d) a fragment of an anti-SARS-CoV-2 antibody light chain comprising at least 80% of the full length sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130.

In one embodiment, the first nucleic acid molecule comprises a nucleotide sequence selected from the group consisting of: a) a nucleotide sequence having at least 80% identity to a nucleotide selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and b) a fragment of a nucleotide sequence comprising at least 80% of the full length sequence selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and the second nucleic acid molecule comprises a nucleotide sequence selected from the group consisting of: c) a nucleotide sequence having at least 80% identity to a nucleotide selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain; and d) a fragment of a nucleotide sequence comprising at least 80% of the full length sequence selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain.

In one embodiment, the composition further comprises a pharmaceutically acceptable excipient.

In one embodiment, the composition further comprises an adjuvant.

In one embodiment the invention relates to a method of preventing or treating a disease in a subject, the method comprising administering to the subject a) an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, b) a nucleic acid molecule encoding an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, c) a composition comprising at least one anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, or d) a composition comprising at least one nucleic acid molecule encoding an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody.

In one embodiment, the disease is COVID-19.

In one embodiment, the method further comprises administering at least one additional SARS-CoV-2 vaccine or therapeutic agent for the treatment of COVID-19 to the subject.

In one embodiment the invention relates to a method of inducing an immune response against SARS-CoV-2 in a subject, the method comprising administering to the subject a) an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, b) a nucleic acid molecule encoding an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, c) a composition comprising at least one anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody, or d) a composition comprising at least one nucleic acid molecule encoding an anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody.

In one embodiment, the invention relates to a method of inducing an immune response against SARS-CoV-2 in a subject in need thereof, the method comprising administering a combination of a first composition comprising a nucleic acid molecule encoding a synthetic anti-SARS-CoV-2 antibody, or fragment thereof wherein the antibody is selected from the group consisting of a monoclonal antibody, a polyclonal antibody, a single chain antibody, an immunoconjugate, a defucosylated antibody, and a bispecific antibody, and a second composition comprising a nucleic acid molecule encoding a SARS-CoV-2 antigen to the subject.

In one embodiment, the nucleic acid molecule encoding the SARS-CoV-2 antigen comprises a nucleotide sequence encoding a peptide comprising an amino acid sequence having at least about 90% identity over an entire length of the amino acid sequence selected from the group consisting of SEQ ID NO:134, SEQ ID NO:136, SEQ ID NO:138 and SEQ ID NO:140.

In one embodiment, the nucleic acid molecule encoding the SARS-CoV-2 antigen comprises a nucleotide sequence encoding the amino acid sequence selected from the group consisting of SEQ ID NO:134, SEQ ID NO:136, SEQ ID NO:138 and SEQ ID NO:140.

In one embodiment, the nucleic acid molecule encoding the SARS-CoV-2 antigen comprises a nucleotide sequence having at least about 90% identity over an entire length of the nucleic acid sequence selected from the group consisting of SEQ ID NO:133, SEQ ID NO:135, SEQ ID NO:137 and SEQ ID NO:139.

In one embodiment, the nucleic acid molecule encoding the SARS-CoV-2 antigen comprises a nucleotide sequence selected from the group consisting of SEQ ID NO:133, SEQ ID NO:135, SEQ ID NO:137 and SEQ ID NO:139.

In one embodiment, administering includes at least one of electroporation and injection.

In one embodiment, the invention relates to a method of treating or protecting a subject in need thereof from infection with SARS-CoV-2 or a disease or disorder associated therewith, the method comprising administering a combination of a first composition comprising a nucleic acid molecule encoding a synthetic anti-SARS-CoV-2 antibody, or fragment thereof wherein the antibody is selected from the group consisting of a monoclonal antibody, a polyclonal antibody, a single chain antibody, an immunoconjugate, a defucosylated antibody, and a bispecific antibody, and a second composition comprising a nucleic acid molecule encoding a SARS-CoV-2 antigen to the subject.

In one embodiment, the disease is COVID-19.

In one embodiment, the nucleic acid molecule encoding the SARS-CoV-2 antigen comprises a nucleotide sequence encoding a peptide comprising an amino acid sequence having at least about 90% identity over an entire length of the amino acid sequence selected from the group consisting of SEQ ID NO:134, SEQ ID NO:136, SEQ ID NO:138 and SEQ ID NO:140.

In one embodiment, the nucleic acid molecule encoding the SARS-CoV-2 antigen comprises a nucleotide sequence encoding the amino acid sequence selected from the group consisting of SEQ ID NO:134, SEQ ID NO:136, SEQ ID NO:138 and SEQ ID NO:140.

In one embodiment, the nucleic acid molecule encoding the SARS-CoV-2 antigen comprises a nucleotide sequence having at least about 90% identity over an entire length of the nucleic acid sequence selected from the group consisting of SEQ ID NO:133, SEQ ID NO:135, SEQ ID NO:137 and SEQ ID NO:139.

In one embodiment, the nucleic acid molecule encoding the SARS-CoV-2 antigen comprises a nucleotide sequence selected from the group consisting of SEQ ID NO:133, SEQ ID NO:135, SEQ ID NO:137 and SEQ ID NO:139.

In one embodiment, administering includes at least one of electroporation and injection.

### BRIEF DESCRIPTION OF THE DRAWINGS

FIG. 1 depicts a diagram of the expression of CR3022 dMAB using a dual-plasmid system.

FIG. 2 depicts exemplary experimental results demonstrating in vitro expression and characterization of the CR3022 dMAB.

FIG. 3 depicts exemplary experimental results demonstrating that the CR3022 DMAb binds to SARS-CoV-2 receptor-binding domain (RBD) in vitro.

FIG. 4 depicts exemplary experimental results demonstrating the in vivo expression kinetics of the CR3022 dMAB.

FIG. 5 depicts exemplary experimental results demonstrating that in vivo-produced CR3022 dMAB binds to recombinant SARSCoV-2 RBD and S1 domains.

FIG. 6 depicts a diagram of the development of SARS-CoV-2 clones for expression of dMABs using a single or dual-plasmid system.

FIG. 7 depicts exemplary experimental results demonstrating the expression and characterization of the S309 DMAb (neutralizing).

FIG. 8 depicts exemplary experimental results demonstrating an in vitro evaluation of the 2130, 2381 and 2196 DMAb variants.

FIG. 9 depicts exemplary experimental results demonstrating the in vivo expression kinetics of the 2130, 2381 and 2196 DMAb variants.

FIG. 10 depicts exemplary experimental results demonstrating the quantification and binding of in vivo-produced 2130, 2381 and 2196 DMAbs.

FIG. 11 depicts exemplary experimental results demonstrating that sera pools from 2130, 2381 and 2196 DMAb-administered mice demonstrate potent neutralization of SARS-CoV-2 pseudovirus.

FIG. 12 depicts exemplary experimental results demonstrating that 2196_MOD DMAb demonstrates in vivo expression and more potent neutralization than the parental 2196 WT DMAb.

FIG. 13A through FIG. 13B depict flowcharts for mAb development. FIG. 13A and FIG. 13B show that Balb/c mice were immunized with a synthetic, consensus sequence of SARS-CoV-2-full length spike DNA as prime. The DNA was injected at the dose of 25 μg per mouse in two-weeks interval and boosting was done by SARS-CoV-2-RBD protein (50 μg/mouse) after the second immunization. The sera from the immunized mouse were collected and then evaluated through ELISA to detect the presence of antibodies against SARS-CoV-2. Upon confirmation, mouse splenocytes were harvested, B lymphocytes were isolated and used to make hybridomas. Finally, positive hybridoma clones were characterized by an indirect ELISA and those selected were further subcloned and expanded for future characterization and analyses.

FIG. 14A through FIG. 14E depict the construction and expression of SARS-CoV-2-Spike full length protein. FIG. 14A depicts a schematic representation of SARS-CoV-2 full length Spike cloned in to pCDNA3.1 vector. FIG. 14B depicts that the SARS-CoV-2 full length Spike protein was expressed in a mammalian system with a Fc tag and Avi tag at the C-terminal and its size was confirmed in a Bis-Tris PAGE gel. FIG. 14C depicts that it exhibited >95% purity as shown by the HPLC. FIG. 14D depicts the validation of SARS-CoV-2 full length protein's specificity by ELISA using the immune sera from SARS-CoV-2-Spike DNA vaccinated mice (n=4). In case of all the mice sera samples, dose dependent binding curves were obtained. m1 through m4 designates 4 different mice for which binding curves were obtained. FIG. 14E depicts a western blot analysis for IgG mAb clones (WCoVA1, WCoVA2, WCoVA3, WCoVA4, WCoVA5, WCoVA6, WCoVA7, WCoVA8, WCoVA9 and WCoVA10) for their expression of heavy and light chain by SDS-PAGE (12%) analysis.

FIG. 15A through FIG. 15D depict the characterization of SARS-CoV-2-Spike IgG mAbs. FIG. 15A depicts an evaluation of IgG clones for their binding potential to SARS-CoV-2-full length Spike and RBD. ELISA plates were coated with recombinant SARS-CoV-2-full length Spike protein (1 μg/ml) as well as RBD (1 μg/ml) and were tested with serially diluted mAb clones as indicated. FIG. 15B depicts serum IgG end point titers of the mAb clones to SARS-CoV-2-full length Spike protein and RBD were determined using end point titer ELISA. Recombinant Avi-Tag protein was used as binding control where no binding of IgG mAb clones was observed. FIG. 15C depicts a binding specificity analysis of the mAb clones against SARS-CoV-2-RBD protein by Western blot. The antibodies were probed with goat anti-mouse IgG-IRDye CW-800 secondary antibody. FIG. 15D depicts the binding kinetics of SARS-CoV-2-Spike mAbs and their target; SARS-CoV-2-RBD by Surface Plasmon Resonance analysis. Sensograms representing the responses of IgG mAb clones which exhibited robust binding to SARS-CoV-2-RBD, against time, showing the progress of their interaction.

FIG. 16 depicts the binding of IgG mAbs to SARS-CoV-2-RBD protein by SPR analysis. Binding level bar graphs showing the binding of IgG mAbs to SARS-CoV-2-RBD protein by SPR analysis using a Biacore T200 SPR system. Y-axis represents the reference subtracted RU signal that was recorded at the end of association phase (300 s).

FIG. 17A through FIG. 17C depict that the SARS-CoV-2-Spike mAbs compete with ACE2 receptor for SARS-CoV-2 Spike protein binding. FIG. 17A depicts that serial dilutions of SARS-CoV-2-Spike IgG mAbs were added to SARS-CoV-2 coated wells prior to the addition of ACE2 protein. ACE2 binding to SARS-CoV-2-Spike mAbs was measured, competition was displayed by SARS-CoV-2-Spike IgG mAbs against ACE2 receptor binding to SARS-CoV-2-Spike protein. FIG. 17B depicts flow cytometry-based receptor binding inhibition; shown is staining of CHO-ACE2 cells with SARS-CoV-2 Spike and flow cytometric analysis. Cells were analyzed for Spike binding (x axis). FIG. 17C depicts the percentages of cells that scored negative or positive are shown in each quadrant.

FIG. 18A through FIG. 18D depict the neutralization efficacy of SARS-CoV-2-Spike mAbs against SARS-CoV-2 pseudovirus assay. The functionalities of the SARS-CoV-2-Spike IgG mAbs were evaluated using a pseudovirus neutralization assay. Neutralization efficacy of (FIG. 18A) SARS-CoV-2 wild type and (FIG. 18B) D614G mutated pseudovirus by SARS-CoV-2 IgG mAb clones represented in terms of % neutralization. FIG. 18C depicts the IC50 values of the SARS-CoV-2-Spike IgG mAb clones, represented in the bar graph. FIG. 18D depicts the correlation between ELISA titers and viral neutralization titers. Statistical analysis was performed in GraphPad Prism. The experiments were performed once. IC50, half-maximum inhibitory concentration.

FIG. 19A through FIG. 19E depict a glycomic analysis of SARS-CoV-2 antibodies reveals a profile compatible with higher Fc-mediated effector functions. FIG. 19A depicts a schematic structure of antibody glycosylation highlighting several monosaccharides and their known impact on Fc-mediated effector functions. FIG. 19B through FIG. 19E depict the percentage of total core fucose (FIG. 19B), terminal sialic acid (FIG. 19C), bisected GlcNac (FIG. 19D), and terminal galactose (FIG. 19E), within the total glycome of five SARS-CoV2 antibodies, bulk IgG from three control C57BL/6 mice, and bulk IgG from a human control sample (ran in triplicate). Median and interquartile range are displayed.

FIG. 20A through FIG. 20F depict computational modeling of antibody docking with SARS-CoV-2-Spike protein. FIG. 20A and FIG. 20D depict structures of WCoVA7 and WCoVA9 variable regions were predicted by AbYmod. FIG. 20B and FIG. 20C depict a docked model of full-length SARS-CoV-2 spike structure (PDB: 6VYB) and WCoVA7 antibody. Left: Cartoon docking model (Green: Full length Spike protein; Red: Antibody variable region). Right: Surface docking model (Gray: Full length Spike protein; Red: Antibody variable region). FIG. 20E and FIG. 20F depict a docked model of full-length SARS-CoV-2 spike structure (PDB: 6VYB) and WCoVA9 antibody. Cartoon docking model (Green: Full length Spike protein; Red: Antibody variable region). Right: Surface docking model (Gray: Full length Spike protein; Red: Antibody variable region).

FIG. 21A and FIG. 21B depict a comparison of SARS-CoV-2, SARS-CoV and MERS-CoV spike glycoproteins. FIG. 21A: Amino acid alignment of coronavirus spike proteins including 11 SARS-CoV-2 sequences with mutations (GISAID). Grey bars indicates identical amino acids and colored bars represent mutations relative to Wuhan-Hu-1. RBD, Cleavage Site, Fusion Peptide and Transmembrane domains are indicated in red. FIG. 21B: Structural models for SARS-CoV-2, SARS and MERS glycoproteins with one chain represented as cartoon and two chains represented as surface. RBD of SARS-CoV-2 is colored yellow.

FIG. 22A through FIG. 22D depict depicts the design and expression of COVID-19 synthetic DNA vaccine constructs. FIG. 22A: Schematic diagram of COVID-19 synthetic DNA vaccine constructs, pGX9501 (matched) and pGX9503 (outlier (OL)) containing the IgE leader sequence and SARS-CoV-2 spike protein insert. FIG. 22B: RT-PCR assay of RNA extract from COS-7 cells transfected with pGX9501 & pGX9503. Extracted RNA was analyzed by RT-PCR using PCR assays designed for each target and for COS-7 β-Actin mRNA, used as an internal expression normalization gene. Delta C_{T} (Δ C_{T}) was calculated as the C_{T} of the target minus the C_{T} of β-Actin for each transfection concentration and is plotted against the log of the mass of pDNA transfected. FIG. 22C: Analysis of in vitro expression of Spike protein after transfection of 293T cells with pGX9501, pGX9503 or MOCK plasmid by Western blot. 293T cell lysates were resolved on a gel and probed with a polyclonal anti-SARS Spike Protein. Blots were stripped then probed with an anti-β-actin loading control. FIG. 22D: In vitro immunofluorescent staining of 293T cells transfected with 3 μg/well of pGX9501, pGX9503 or pVax (empty control vector). Expression of Spike protein was measured with polyclonal anti-SARS Spike Protein IgG and anti-IgG secondary (green). Cell nuclei were counterstained with DAPI (blue). Images were captured using ImageXpress Pico automated cell imaging system.

FIG. 23 depicts an IgG binding screen of a panel of SARS-CoV-2 and SARS-CoV antigens using serum from INO-4800-treated mice. BALB/c mice were immunized on Day 0 with 25 μg INO-4800 or pVAX-empty vector (Control) as described in the methods. Protein antigen binding of IgG at 1:50 and 1:250 serum dilutions from mice at day 14. Data shown represent mean OD450 nm values (mean+SD) for each group of 4 mice.

FIG. 24A through FIG. 24D depict the humoral responses to SARS-CoV-2 S 1+2 and S RBD protein antigen in BALB/c mice after a single dose of INO-4800 BALB/c mice were immunized on day 0 with indicated doses of INO-4800 or pVAX-empty vector as described in the methods. (FIG. 4A) SARS-CoV-2 S1+2 or (FIG. 4C) SARS-CoV-2 RBD protein antigen binding of IgG in serial serum dilutions from mice at day 14. Data shown represent mean OD450 nm values (mean+SD) for each group of 8 mice (FIG. 24A and FIG. 24B) and 5 mice (FIG. 24C and FIG. 24D). Serum IgG binding endpoint titers to (FIG. 24B) SARS-CoV-2 S1+2 and (FIG. 24D) SARS-CoV-2 RBD protein. Data representative of 2 independent experiments.

FIG. 25A through FIG. 25C depict serum IgG from INO-4800 immunized mice compete with ACE2 receptor for SARS-CoV-2 Spike protein binding. FIG. 25A: Soluble ACE2 receptor binds to CoV-2 full-length spike with an EC₅₀ of 0.025 μg/ml. FIG. 25B: Purified serum IgG from BALB/c mice after second immunization with INO-4800 yields significant competition against ACE2 receptor. Serum IgG samples from the animals were run in triplicate. FIG. 25C: IgGs purified from n=5 mice day 14 post second immunization with INO-4800 show significant competition against ACE2 receptor binding to SARS-CoV-2 S 1+2 protein. The soluble ACE2 concentration for the competition assay is ˜0.1 μg/ml.

FIG. 26 depicts IgGs purified from n=5 mice day 14 post second immunization with INO-4800 show competition against ACE2 receptor binding to SARS-CoV-2 Spike protein compared to pooled naïve mice IgGs. Naïve mice were run in a single column. Vaccinated mice were run in duplicate. If error bars are not visible, error is smaller than the data point.

FIG. 27A and FIG. 27B depict humoral responses to SARS-CoV-2 in Hartley guinea pigs after a single dose of INO-4800. Hartley guinea pigs mice were immunized on Day 0 with 100 μg INO-4800 or pVAX-empty vector as described in the methods. FIG. 27A: SARS-CoV-2 S protein antigen binding of IgG in serial serum dilutions at day 0 and 14. Data shown represent mean OD450 nm values (mean+SD) for the 5 guinea pigs. FIG. 27B: Serum IgG binding titers (mean±SD) to SARS-CoV-2 S protein at day 14. P=0.0079, Mann-Whitney test.

FIG. 28A and FIG. 28B depict serum from INO-4800 immunized guinea pigs mediates the inhibition of ACE-2 binding to SARS-CoV-2 S protein. Hartley guinea pigs were immunized on Day 0 and 14 with 100 μg INO-4800 or pVAX-empty vector as described in the methods. FIG. 28A: Day 28 collected serum (diluted 1:20) was added SARS-CoV-2 coated wells prior to the addition of serial dilutions of ACE-2 protein. Detection of ACE-2 binding to SARS-CoV-2 S protein was measured. Sera collected from 5 INO-4800-treated and 3 pVAX-treated animals were used in this experiment. FIG. 28B: Serial dilutions of guinea pig serum collected on day 21 were added to SARS-CoV-2 coated wells prior to the addition of ACE-2 protein. Detection of ACE-2 binding to SARS-CoV-2 S protein was measured. Sera collected from 4 INO-4800-treated and 5 pVAX-treated guinea pigs was used in this experiment.

FIG. 29A through FIG. 29D depict detection of SARS-CoV-2 S protein-reactive antibodies in the BAL of INO-4800 immunized animals. BALB/c mice were immunized on days 0 and 14 with INO-4800 or pVAX and BAL collected at day 21 (FIG. 29A and FIG. 29B). Hartley guinea pigs were immunized on days 0, 14 and 21 with INO-4800 or pVAX and BAL collected at day 42 (FIG. 29C and FIG. 29D). Bronchoalveolar lavage fluid was assayed for SARS-CoV-2 Spike protein-specific antibodies by ELISA. Data are presented as endpoint titers (FIG. 29A and FIG. 29C), and BAL dilution curves with raw OD 450 nm values (FIG. 29B and FIG. 29D). (FIG. 29A and FIG. 29C) bars represent the average of each group and error bars the standard deviation. **p<0.01 by Mann-Whitney U test. Data are representative of one experiment for each species with n=5/group

FIG. 30A through FIG. 30C depict rapid induction of T cell responses in BALB/c mice post-administration of INO-4800. BALB/c mice (n=5/group) were immunized with 2.5 or 10 μg INO-4800. T cell responses were analyzed in the animals on days 4, 7, 10 for FIG. 30A and FIG. 30B, and day 14 for FIG. 30C. T cell responses were measured by IFN-γ ELISpot in splenocytes stimulated for 20 hours with overlapping peptide pools spanning the SARS-CoV-2 (FIG. 30A), SARS-CoV (FIG. 30B), or MERS-CoV (FIG. 30C) Spike proteins. Bars represent the mean+SD.

FIG. 31A through FIG. 31F depict ELISpot images of IFN-γ+ mouse splenocytes after stimulation with SARS-CoV-2 and SARS antigens. Mice were immunized on day 0 and splenocytes harvested at the indicated time points. IFNγ-secreting cells in the spleens of immunized animals were enumerated via ELISpot assay. Representative images show SARS-CoV-2 specific (FIG. 31A through FIG. 31C) and SARS-CoV-specific (FIG. 31D through FIG. 31F) IFNγ spot forming units in the splenocyte population at days 4, 7, and 10 post-immunization. Images were captured by ImmunoSpot CTL reader.

FIG. 32A and FIG. 32B depict flow cytometric analysis of T cell populations producing IFN-γ upon SARS-CoV-2 S protein stimulation. Splenocytes harvested from BALB/c and C57BL/6 mice 14 days after pVAX or INO-4800 treatment were made into single cell suspensions. The cells were stimulated for 6 hours with SARS-CoV-2 overlapping peptide pools. FIG. 32A: CD4+ and CD8+ T cell gating strategy; singlets were gated on (i), then lymphocytes (ii) followed by live CD45+ cells (iii). Next CD3+ cells were gated (iv) and from that population CD4+ (v) and CD8+ (vi) T-cells were gated. IFNγ+ cells were gated from each of the CD4+ (vii) and CD8+ (viii) T-cell populations. FIG. 32B: The percentage of CD4+ and CD8+ T cells producing IFNγ is depicted. Bars represent mean+SD. 4 BALB/c and 4 C57BL/6 mice were used in this study. *p<0.05, Mann Whitney test.

FIG. 33A and FIG. 33B depict T cell epitope mapping after INO-4800 administration to BALB/c mice. Splenocytes were stimulated for 20 hours with SARS-CoV-2 peptide matrix pools. FIG. 33A: T cell responses following stimulation with matrix mapping SARS-CoV-2 peptide pools. Bars represent the mean+SD. FIG. 33B: Map of the SARS-CoV-2 Spike protein and identification of immunodominant peptides in BALB/c mice. A known immunodominant SARS-CoV HLA-A2 epitope is included for comparison.

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, RSV-LTR promoter, tac promoter, SV40 early promoter or SV40 late promoter and the CMV IE promoter.

“Sample” or “biological sample” as used herein means a biological material isolated from an individual. The biological sample may contain any biological material suitable for detecting the desired biomarkers, and may comprise cellular and/or non-cellular material obtained from the individual.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof, (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITIONS

In some embodiments, the invention provides compositions that bind to SARS-CoV-2 antigen, including, but not limited to, a SARS-CoV-2 spike protein. In some embodiments, the composition that binds to SARS-CoV-2 spike protein is an antibody.

The instant invention relates to the design and development of a synthetic DNA plasmid-encoding human anti-SARS-CoV-2 monoclonal antibody sequences as a novel approach to immunotherapy of SARS-CoV-2 infection, or COVID-19. A single inoculation with this anti-SARS-CoV-2-DMAb generates functional anti-SARS-CoV-2 activity for several weeks in the serum of inoculated animals. Anti-SARS-CoV-2 DMAbs can function as an immune-prophylaxis strategy for SARS-CoV-2 infection, or COVID-19.

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

In one embodiment, the nucleic acid molecule comprises a nucleotide sequence encoding an antibody to the receptor binding domain (RBD) or the Spike protein of the SARS-CoV-2 virus (anti-SARS-CoV-2).

In one embodiment, the nucleic acid molecule comprises a nucleotide sequence encoding a variable heavy chain region and a nucleotide sequence encoding a variable light chain region of an anti-SARS-CoV-2 antibody.

In one embodiment, the invention provides a composition comprising a first nucleic acid molecule comprising a nucleotide sequence encoding a variable heavy chain region of an anti-SARS-CoV-2 antibody and a second nucleic acid molecule comprising a nucleotide sequence encoding a variable light chain region of an anti-SARS-CoV-2 antibody.

Antibodies, including SARS-CoV-2 spike protein fragments, of the present invention include, in certain embodiments, antibody amino acid sequences disclosed herein encoded by any suitable polynucleotide, or any isolated or formulated antibody. Further, antibodies of the present disclosure comprise antibodies having the structural and/or functional features of anti-SARS-CoV-2 spike protein antibodies described herein. In one embodiment, the anti-SARS-CoV-2 spike protein antibody binds SARS-CoV-2 spike protein and, thereby partially or substantially alters at least one biological activity of SARS-CoV-2 spike protein (e.g., receptor binding activity).

In one embodiment, anti-SARS-CoV-2 spike protein antibodies of the invention immunospecifically bind at least one specified epitope specific to the SARS-CoV-2 spike protein and do not specifically bind to other polypeptides. The at least one epitope can comprise at least one antibody binding region that comprises at least one portion of the SARS-CoV-2 spike protein. The term “epitope” as used herein refers to a protein determinant capable of binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

In some embodiments, the invention includes compositions comprising an antibody that specifically binds to SARS-CoV-2 spike protein (e.g., binding portion of an antibody). In one embodiment, the anti-SARS-CoV-2 spike protein antibody is a polyclonal antibody. In another embodiment, the anti-SARS-CoV-2 spike protein antibody is a monoclonal antibody. In some embodiments, the anti-SARS-CoV-2 spike protein antibody is a chimeric antibody. In further embodiments, the anti-SARS-CoV-2 spike protein antibody is a humanized antibody.

The binding portion of an antibody comprises one or more fragments of an antibody that retain the ability to specifically bind to binding partner molecule (e.g., SARS-CoV-2 spike protein). It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “binding portion” of an antibody. These antibody fragments are obtained using conventional techniques known to those with skill in the art, and the fragments are screened for utility in the same manner as are intact antibodies. Binding portions can be produced by recombinant DNA techniques, or by enzymatic or chemical cleavage of intact immunoglobulins.

An antibody that binds to SARS-CoV-2 spike protein of the invention is an antibody that inhibits, blocks, or interferes with at least one SARS-CoV-2 spike protein activity (e.g., receptor binding activity), in vitro, in situ and/or in vivo.

In one embodiment, the SARS-CoV-2 antibody comprises a heavy chain comprising an amino acid sequence as set forth in SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 or SEQ ID NO:128. In one embodiment, the SARS-CoV-2 antibody comprises a light chain comprising an amino acid sequence as set forth in SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 or SEQ ID NO:130.

Given that certain of the monoclonal antibodies can bind to the SARS-CoV-2 spike protein, the VH and VL sequences can be “mixed and matched” to create other anti-SARS-CoV-2 spike protein binding molecules of this disclosure. Binding of such “mixed and matched” antibodies can be tested using standard binding assays known in the art (e.g., immunoblot etc.). In some embodiments, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence.

Accordingly, in one aspect, this disclosure provides an isolated monoclonal antibody, or binding portion thereof comprising: (a) a heavy chain variable region comprising an amino acid sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 or SEQ ID NO:128; and (b) a light chain variable region comprising an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 or SEQ ID NO:130 wherein the antibody specifically binds a SARS-CoV-2 spike protein.

In some embodiments, the heavy and light chain combinations include: (a) a heavy chain variable region comprising the amino acid sequence of SEQ ID NO: 2 and a light chain variable region comprising the amino acid sequence of SEQ ID NO: 4; (b) a heavy chain variable region comprising the amino acid sequence of SEQ ID NO: 6 and a light chain variable region comprising the amino acid sequence of SEQ ID NO: 8; (c) a heavy chain variable region comprising the amino acid sequence of SEQ ID NO: 12 and a light chain variable region comprising the amino acid sequence of SEQ ID NO: 14; (d) a heavy chain variable region comprising the amino acid sequence of SEQ ID NO: 18 and a light chain variable region comprising the amino acid sequence of SEQ ID NO: 20; (e) a heavy chain variable region comprising the amino acid sequence of SEQ ID NO: 30 and a light chain variable region comprising the amino acid sequence of SEQ ID NO: 38; (f) a heavy chain variable region comprising the amino acid sequence of SEQ ID NO: 42 and a light chain variable region comprising the amino acid sequence of SEQ ID NO: 44; (g) a heavy chain variable region comprising the amino acid sequence of SEQ ID NO: 84 and a light chain variable region comprising the amino acid sequence of SEQ ID NO: 92; (h) a heavy chain variable region comprising the amino acid sequence of SEQ ID NO: 94 and a light chain variable region comprising the amino acid sequence of SEQ ID NO: 96; (i) a heavy chain variable region comprising the amino acid sequence of SEQ ID NO: 100 and a light chain variable region comprising the amino acid sequence of SEQ ID NO: 102; (j) a heavy chain variable region comprising the amino acid sequence of SEQ ID NO: 112 and a light chain variable region comprising the amino acid sequence of SEQ ID NO: 120; (k) a heavy chain variable region comprising the amino acid sequence of SEQ ID NO: 122 and a light chain variable region comprising the amino acid sequence of SEQ ID NO: 124; or (l) a heavy chain variable region comprising the amino acid sequence of SEQ ID NO: 128 and a light chain variable region comprising the amino acid sequence of SEQ ID NO: 130.

Given that each of these antibodies can bind to SARS-CoV-2 spike protein and that binding specificity is provided primarily by the CDRl, CDR2, and CDR3 regions, the VH CDRl, CDR2, and CDR3 sequences and VL CDR1, CDR2, and CDR3 sequences can be “mixed and matched” to create other anti-SARS-CoV-2 spike protein binding molecules of this disclosure. SARS-CoV-2 spike protein binding of such “mixed and matched” antibodies can be tested using standard binding assays known in the art (e.g., immunoblot). Preferably, when VH CDR sequences are mixed and matched, the CDRl, CDR2 and/or CDR3 sequence from a particular VH sequence is replaced with a structurally similar CDR sequence(s). Likewise, when VL CDR sequences are mixed and matched, the CDRl, CDR2 and/or CDR3 sequence from a particular VL sequence preferably is replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences disclosed herein.

Accordingly, in another aspect, the invention provides an isolated monoclonal antibody, or binding portion thereof comprising at least one selected from: (a) a heavy chain variable region CDRl comprising an amino acid sequence of SEQ ID NO:26, SEQ ID NO: 80 or SEQ ID NO:108; (b) a heavy chain variable region CDR2 comprising an amino acid sequence of SEQ ID NO:27, SEQ ID NO: 81 or SEQ ID NO:109; (c) a heavy chain variable region CDR3 comprising an amino acid sequence of SEQ ID NO:28, SEQ ID NO: 82 or SEQ ID NO:110; (d) a light chain variable region CDRl comprising an amino acid sequence of SEQ ID NO:34, SEQ ID NO: 88 or SEQ ID NO:116; (e) a light chain variable region CDR2 comprising an amino acid sequence of SEQ ID NO:35, SEQ ID NO: 89 or SEQ ID NO:117; and (f) a light chain variable region CDR3 comprising an amino acid sequence of SEQ ID NO:36, SEQ ID NO: 90 or SEQ ID NO:118; wherein the antibody specifically binds SARS-CoV-2 spike protein.

In another embodiment, the antibody comprises (a) a heavy chain variable region CDRl comprising SEQ ID NO: 26; (b) a heavy chain variable region CDR2 comprising SEQ ID NO: 27; (c) a heavy chain variable region CDR3 comprising SEQ ID NO: 28; (d) a light chain variable region CDRl comprising SEQ ID NO: 34; (e) a light chain variable region CDR2 comprising SEQ ID NO: 35 and (f) a light chain variable region CDR3 comprising SEQ ID NO: 36.

In another embodiment, the antibody comprises (a) a heavy chain variable region CDRl comprising SEQ ID NO: 80; (b) a heavy chain variable region CDR2 comprising SEQ ID NO: 81; (c) a heavy chain variable region CDR3 comprising SEQ ID NO: 82; (d) a light chain variable region CDRl comprising SEQ ID NO: 88; (e) a light chain variable region CDR2 comprising SEQ ID NO: 89 and (f) a light chain variable region CDR3 comprising SEQ ID NO: 90.

In another embodiment, the antibody comprises (a) a heavy chain variable region CDRl comprising SEQ ID NO: 109; (b) a heavy chain variable region CDR2 comprising SEQ ID NO: 110; (c) a heavy chain variable region CDR3 comprising SEQ ID NO: 111; (d) a light chain variable region CDRl comprising SEQ ID NO: 116; (e) a light chain variable region CDR2 comprising SEQ ID NO: 117 and (f) a light chain variable region CDR3 comprising SEQ ID NO: 118.

The foregoing isolated anti-SARS-CoV-2 spike protein antibody CDR sequences establish a novel family of SARS-CoV-2 spike protein binding proteins, isolated in accordance with this invention, and comprising polypeptides that include the CDR sequences listed. To generate and to select CDR's of the invention having SARS-CoV-2 spike protein binding and/or SARS-CoV-2 spike protein detection and/or SARS-CoV-2 spike protein neutralization activity, standard methods known in the art for generating binding proteins of the present invention and assessing the binding and/or detection and/or neutralizing characteristics of those binding protein may be used, including but not limited to those specifically described herein.

In one embodiment, anti-SARS-CoV-2 antibody comprises an amino acid sequence having at least 60%, 70%, 80%, 90%, 95%, 96%, 97%, 98%, or 99% identity to an amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132, or a combination thereof. In one embodiment, the nucleotide sequence encoding an anti-SARS-CoV-2 antibody comprises a nucleic acid sequence encoding SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132, or a combination thereof. In one embodiment, the nucleotide sequence encoding an anti-SARS-CoV-2 antibody comprises a codon optimized nucleic acid sequence encoding a fragment comprising at least 60%, 70%, 80%, 90%, 95%, 96%, 97%, 98%, or 99% of the full length of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132, or a combination thereof.

In one embodiment, the nucleotide sequence encoding an anti-SARS-CoV-2 antibody comprises an RNA sequence transcribed from a DNA sequence encoding an amino acid sequence having at least 60%, 70%, 80%, 90%, 95%, 96%, 97%, 98%, or 99% identity to an amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132, or a combination thereof. In one embodiment, the nucleotide sequence encoding an anti-SARS-CoV-2 antibody comprises an RNA sequence transcribed from a DNA sequence encoding an amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132. In one embodiment, the nucleotide sequence encoding an anti-SARS-CoV-2 antibody comprises an RNA sequence transcribed from a DNA sequence encoding a fragment comprising at least 60%, 70%, 80%, 90%, 95%, 96%, 97%, 98%, or 99% of the full length of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:30, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:84, SEQ ID NO:92, SEQ ID NO:94, SEQ ID NO:96, SEQ ID NO:98, SEQ ID NO:100, SEQ ID NO:102, SEQ ID NO:104, SEQ ID NO:112, SEQ ID NO:120, SEQ ID NO:122, SEQ ID NO:124, SEQ ID NO:126, SEQ ID NO:128, SEQ ID NO:130 or SEQ ID NO:132, or a combination thereof.

In one embodiment, the nucleotide sequence encoding an anti-SARS-CoV-2 antibody comprises a sequence having at least 60%, 70%, 80%, 90%, 95%, 96%, 97%, 98%, or 99% identity to a nucleic acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131 or a combination thereof. In one embodiment, the nucleotide sequence encoding an anti-SARS-CoV-2 antibody comprises a DNA sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131, or a combination thereof. In one embodiment, the nucleotide sequence encoding an anti-SARS-CoV-2 antibody comprises a fragment comprising at least 60%, 70%, 80%, 90%, 95%, 96%, 97%, 98%, or 99% of the full length of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131, or a combination thereof.

In one embodiment, the nucleotide sequence encoding an anti-SARS-CoV-2 antibody comprises an RNA sequence transcribed from a codon optimized nucleotide sequences having at least 60%, 70%, 80%, 90%, 95%, 96%, 97%, 98%, or 99% identity to a nucleic acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131, or a combination thereof. In one embodiment, the nucleotide sequence encoding an anti-SARS-CoV-2 antibody comprises an RNA sequence transcribed from a DNA sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131, or a combination thereof. In one embodiment, the nucleotide sequence encoding an anti-SARS-CoV-2 antibody comprises an RNA sequence transcribed from a fragment comprising at least 60%, 70%, 80%, 90%, 95%, 96%, 97%, 98%, or 99% of the full length of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:29, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:83, SEQ ID NO:91, SEQ ID NO:93, SEQ ID NO:95, SEQ ID NO:97, SEQ ID NO:99, SEQ ID NO:101, SEQ ID NO:103, SEQ ID NO:111, SEQ ID NO:119, SEQ ID NO:121, SEQ ID NO:123, SEQ ID NO:125, SEQ ID NO:127, SEQ ID NO:129 or SEQ ID NO:131, or a combination thereof.

In one embodiment, the invention relates to a combination of a first nucleic acid molecule encoding a heavy chain of an anti-SARS-CoV-2 antibody, and a second nucleic acid molecule encoding a light chain of an anti-SARS-CoV-2 antibody. In one embodiment, the first nucleic acid molecule is a first plasmid comprising a nucleotide sequence encoding a heavy chain of an anti-SARS-CoV-2 antibody and the second nucleic acid molecule is a second plasmid encoding a light chain of an anti-SARS-CoV-2 antibody.

In one embodiment, the first nucleic acid molecule encoding a heavy chain of an anti-SARS-CoV-2 antibody encodes SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 or SEQ ID NO:128, or an amino acid sequence having at least 60%, 70%, 80%, 90%, 95%, 96%, 97%, 98%, or 99% identity to an amino acid sequence as set forth in SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 or SEQ ID NO:128 or a fragment comprising at least 60%, 70%, 80%, 90%, 95%, 96%, 97%, 98%, or 99% of the full length of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 or SEQ ID NO:128. In one embodiment, the first nucleic acid molecule encoding a heavy chain of an anti-SARS-CoV-2 antibody comprises a nucleotide sequence of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 or SEQ ID NO:127, a nucleotide sequence having at least 60%, 70%, 80%, 90%, 95%, 96%, 97%, 98%, or 99% identity to SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 or SEQ ID NO:127, or a fragment comprising at least 60%, 70%, 80%, 90%, 95%, 96%, 97%, 98%, or 99% of the full length of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 or SEQ ID NO:127.

In one embodiment, the second nucleic acid molecule encoding a light chain of an anti-SARS-CoV-2 antibody encodes SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 or SEQ ID NO:130, an amino acid sequence having at least 60%, 70%, 80%, 90%, 95%, 96%, 97%, 98%, or 99% identity to an amino acid sequence as set forth in SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 or SEQ ID NO:130 or a fragment comprising at least 60%, 70%, 80%, 90%, 95%, 96%, 97%, 98%, or 99% of the full length of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 or SEQ ID NO:130. In one embodiment, the second nucleic acid molecule encoding a light chain of an anti-SARS-CoV-2 antibody comprises a nucleotide sequence of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 or SEQ ID NO:129, a nucleotide sequence having at least 60%, 70%, 80%, 90%, 95%, 96%, 97%, 98%, or 99% identity to SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 or SEQ ID NO:129 or a fragment comprising at least 60%, 70%, 80%, 90%, 95%, 96%, 97%, 98%, or 99% of the full length of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 or SEQ ID NO:129.

The composition of the invention can treat, prevent and/or protect against any disease, disorder, or condition associated with SARS-CoV-2 infection. In certain embodiments, the composition can treat, prevent, and or/protect against viral infection. In certain embodiments, the composition can treat, prevent, and or/protect against condition associated with SARS-CoV-2 infection. In certain embodiments, the composition can treat, prevent, and or/protect against COVID-19.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

In some embodiments, the SARS-CoV-2 spike protein binding molecules (e.g., antibodies, etc.) of the present invention, exhibit a high capacity to detect and bind SARS-CoV-2 spike protein in a complex mixture of salts, compounds and other polypeptides, e.g., as assessed by any one of several in vitro and in vivo assays known in the art. The skilled artisan will understand that the SARS-CoV-2 spike protein binding molecules (e.g., antibodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the SARS-CoV-2 spike protein binding molecules (e.g., antibodies, etc.) of the present invention, exhibit a high capacity to reduce or to neutralize SARS-CoV-2 spike protein activity (e.g., receptor binding activity, etc.) as assessed by any one of several in vitro and in vivo assays known in the art. For example, these SARS-CoV-2 spike protein binding molecules (e.g., antibodies, etc.) neutralize SARS-CoV-2-associated or SARS-CoV-2-mediated disease or disorder.

As used herein, a SARS-CoV-2 spike protein binding molecule (e.g., antibody, etc.) that “specifically binds to a SARS-CoV-2 spike protein” binds to a SARS-CoV-2 spike protein with a KD of 1×10⁻⁶ M or less, more preferably 1×10⁻⁷ M or less, more preferably 1×10⁻⁸ M or less, more preferably 5×10⁻⁹ M or less, more preferably 1×10⁻⁹ M or less or even more preferably 3×10⁻¹⁰ M or less. The term “does not substantially bind” to a protein or cells, as used herein, means does not bind or does not bind with a high affinity to the protein or cells, i.e., binds to the protein or cells with a KD of greater than 1×10⁶ M or more, more preferably 1×10⁵ M or more, more preferably 1×10⁴ M or more, more preferably 1×10³ M or more, even more preferably 1×10² M or more. The term “KD”, as used herein, is intended to refer to the dissociation constant, which is obtained from the ratio of Kd to Ka (i.e., Kd/Ka) and is expressed as a molar concentration (M). KD values for a SARS-CoV-2 spike protein binding molecule (e.g., antibody, etc.) can be determined using methods well established in the art. A preferred method for determining the KD of a binding molecule (e.g., antibody, etc.) is by using surface plasmon resonance, preferably using a biosensor system such as a Biacore® system.

As used herein, the term “high affinity” for an IgG antibody refers to an antibody having a KD of 1×lO⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target binding partner molecule. However, “high affinity” binding can vary for other antibody isotypes. For example, “high affinity” binding for an IgM isotype refers to an antibody having a KD of 10⁻⁶ M or less, more preferably 10⁻⁷ M or less, even more preferably 10⁻⁸ M or less.

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

### Recombinant Nucleic Acid Sequence

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence for increased translation; addition of an immunoglobulin (Ig) leader sequence encoding a signal peptide; addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

(1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

(2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

(3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

(4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

(5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

(6) Intron

The recombinant nucleic acid sequence construct can include one or more introns. Each intron can include functional splice donor and acceptor sites. The intron can include an enhancer of splicing. The intron can include one or more signals required for efficient splicing.

(7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

(8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

(9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

(10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

(11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

(12) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide. The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

(13) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

(14) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

(15) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extra-chromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

(16) RNA

In one embodiment, the nucleic acid is an RNA molecule. In one embodiment, the RNA molecule is transcribed from a DNA sequence described herein. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

(17) Circular and Linear Vector

The one or more vectors may be circular plasmid, which may transform a target cell by integration into the cellular genome or exist extra-chromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

(18) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

(19) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 3. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

In one embodiment, the antibody binds a SARS-CoV-2 antigen. In one embodiment, the antibody binds at least one epitope of a SARS-CoV-2 Spike protein. In one embodiment, the antibody binds a SARS-CoV-2 RBD.

The antibody may be modified to reduce or prevent antibody-dependent enhancement (ADE) of disease associated with the antigen as described in more detail below.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments one of the binding sites is capable of binding a tumor associated antigen. In some embodiments, the binding site included in the Fab fragment is a binding site specific for a SARS-CoV-2 antigen. In some embodiments, the binding site included in the single chain Fv fragment is a binding site specific for a SARS-CoV-2 antigen such as a SARS-CoV-2 spike antigen.

In some embodiments, one of the binding sites of a bispecific antibody according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing α- and β-chains, in some embodiments it encompasses γ-chains and δ-chains. Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the bispecific antibody molecule binds a SARS-CoV-2 antigen and the second binding site binds a T cell specific receptor molecule and/or a natural killer (NK) cell specific receptor molecule.

In some embodiments, the first binding site of the antibody molecule binds the SARS-CoV-2 spike antigen, and the second binding site binds a T cell specific receptor molecule and/or a natural killer (NK) cell specific receptor molecule. In some embodiments, the first binding site of the antibody molecule binds a SARS-CoV-2 spike antigen and the second binding site binds one of CD3, the T cell receptor (TCR), CD28, CD16, NKG2D, Ox40, 4-1BB, CD2, CD5 and CD95.

In some embodiments, the first binding site of the antibody molecule binds a T cell specific receptor molecule and/or a natural killer (NK) cell specific receptor molecule and the second binding site binds a SARS-CoV-2 antigen. In some embodiments, the first binding site of the antibody binds a T cell specific receptor molecule and/or a natural killer (NK) cell specific receptor molecule and the second binding site binds the SARS-CoV-2 spike antigen. In some embodiments, the first binding site of the antibody binds one of CD3, the T cell receptor (TCR), CD28, CD16, NKG2D, Ox40, 4-1BB, CD2, CD5 and CD95, and the second binding site binds the SARS-CoV-2 spike antigen.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 4. ANTIGEN

The synthetic antibody is directed to the antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a virus. The antigen can be associated with viral infection. In one embodiment, the antigen can be associated with SARS-CoV-2 infection, or COVID-19. In one embodiment, the antigen can be a SARS-CoV-2 spike antigen.

In one embodiment, the antigen can be a fragment of a SARS-CoV-2 antigen. For example, in one embodiment, the antigen is a fragment of a SARS-CoV-2 spike protein. In one embodiment, the antigen is the receptor binding domain (RBD) of the SARS-CoV-2 spike protein.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

### Viral Antigens

The viral antigen can be a viral antigen or fragment or variant thereof. The virus can be a disease causing virus. The virus can be a coronavirus. The virus can be SARS or the SARS-CoV-2 virus.

The antigen may be a SARS-CoV-2 viral antigen, or fragment thereof, or variant thereof. The SARS-CoV-2 antigen can be from a factor that allows the virus to replicate, infect or survive. Factors that allow a SARS-CoV-2 virus to replicate or survive include, but are not limited to, structural proteins and non-structural proteins. Such a protein can be a spike protein.

### 5. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligrams. In some preferred embodiments, composition according to the present invention comprises about 5 nanograms to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 6. METHODS OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 7. METHODS OF IDENTIFYING OR SCREENING FOR THE ANTIBODY

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 8. METHODS OF DIAGNOSING A DISEASE OR DISORDER

The present invention further relates to a method of diagnosing a subject as having a disease or disorder using an antibody, fragment thereof, or nucleic acid molecule encoding the same as described herein. In some embodiments, the present invention features methods for identifying subjects who are at risk of spreading SARS-CoV-2 infection or COVID-19, including those subjects who are asymptomatic or only exhibit non-specific indicators of SARS-CoV-2 infection or COVID-19. In some embodiments, the present invention is also useful for monitoring subjects undergoing treatments and therapies for SARS-CoV-2 infection or COVID-19, and for selecting or modifying therapies and treatments that would be efficacious in subjects having SARS-CoV-2 infection or COVID-19, wherein selection and use of such treatments and therapies promote immunity to SARS-CoV-2, or prevent infection by SARS-CoV-2.

In one embodiment, the antibody, fragment thereof, or nucleic acid molecule encoding the same can be used in an immunoassay for diagnosing a subject as having an active SARS-CoV-2 infection, having COVID-19, or having immunity to SARS-CoV-2 infection, or for monitoring subjects undergoing treatments and therapies for SARS-CoV-2 infection or COVID-19. Non-limiting exemplary immunoassays include, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY.

In some embodiments the methods include obtaining a sample from a subject and contacting the sample with an antibody of the invention or a cell expressing an antibody of the invention and detecting binding of the antibody to an antigen present in the sample.

In some embodiments, samples can be provided from a subject undergoing treatment regimens or therapeutic interventions, e.g., drug treatments, vaccination, etc. for SARS-CoV-2 infection or COVID-19. Samples can be obtained from the subject at various time points before, during, or after treatment.

The SARS-CoV-2 antibodies of the present invention, or nucleic acid molecules encoding the same, can thus be used to generate a risk profile or signature of subjects: (i) who are expected to have immunity to SARS-CoV-2 infection or COVID-19 and/or (ii) who are at risk of developing SARS-CoV-2 infection or COVID-19. The antibody profile of a subject can be compared to a predetermined or reference antibody profile to diagnose or identify subjects at risk for developing SARS-CoV-2 infection or COVID-19, to monitor the progression of disease, as well as the rate of progression of disease, and to monitor the effectiveness of SARS-CoV-2 infection or COVID-19 treatments. Data concerning the antibodies of the present invention can also be combined or correlated with other data or test results for SARS-CoV-2 infection or COVID-19, including but not limited to age, weight, BMI, imaging data, medical history, smoking status and any relevant family history.

The present invention also provides methods for identifying agents for treating SARS-CoV-2 infection or COVID-19 that are appropriate or otherwise customized for a specific subject. In this regard, a test sample from a subject, exposed to a therapeutic agent, drug, or other treatment regimen, can be taken and the level of one or more SARS-CoV-2 antibody can be determined. The level of one or more SARS-CoV-2 antibody can be compared to a sample derived from the subject before and after treatment, or can be compared to samples derived from one or more subjects who have shown improvements in risk factors as a result of such treatment or exposure.

In one embodiment, the invention is a method of diagnosing SARS-CoV-2 infection or COVID-19. In one embodiment, the method includes determining immunity to infection or reinfection by SARS-CoV-2. In some embodiments, these methods may utilize at least one biological sample (such as urine, saliva, blood, serum, plasma, amniotic fluid, or tears), for the detection of one or more SARS-CoV-2 antibody of the invention in the sample. Frequently the sample is a “clinical sample” which is a sample derived from a patient. In one embodiment, the biological sample is a blood sample.

In one embodiment, the method comprises detecting one or more SARS-CoV-2 antigen in at least one biological sample of the subject. In various embodiments, the level of one or more SARS-CoV-2 antigen of the invention in the biological sample of the subject is compared to a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

### 9. METHODS OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not be limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 10. METHODS OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

In certain embodiments, the invention provides a method of treating protecting against, and/or preventing a SARS-CoV-2 virus infection. In one embodiment, the method treats, protects against, and/or prevents a disease or disorder associated with SARS-CoV-2 virus infection. In one embodiment, the method treats, protects against, and/or prevents COVID-19.

In one embodiment the subject has, or is at risk of, SARS-CoV-2 virus infection.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The synthetic antibody can treat, prevent, and/or protect against disease in the subject administered the composition. The synthetic antibody by binding the antigen can treat, prevent, and/or protect against disease in the subject administered the composition. The synthetic antibody can promote survival of the disease in the subject administered the composition. In one embodiment, the synthetic antibody can provide increased survival of the disease in the subject over the expected survival of a subject having the disease who has not been administered the synthetic antibody. In various embodiments, the synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the synthetic antibody can provide increased protection against the disease in the subject over the expected protection of a subject who has not been administered the synthetic antibody. In various embodiments, the synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

In one embodiment, immunotherapy with the anti-SARS-CoV-2 DMAb of the invention will have a direct therapeutic effect. In one embodiment, immunotherapy with the anti-SARS-CoV-2 DMAb of the invention can be used as immune “adjuvant” treatment to reduce viral protein load, in order to provide host immunity and optimize the effect of antiviral drugs.

### 11. COMBINATION WITH SARS-COV-2 ANTIGEN

In one embodiment, the invention relates to the administration of a SARS-CoV-2 antibody or a nucleic acid encoded SARS-CoV-2 antibody in combination with a nucleic acid molecule encoding a SARS-CoV-2 antigen. In some embodiments therefore, the invention relates to immunogenic compositions, such as vaccines, comprising a SARS-CoV-2 antibody or a nucleic acid encoded SARS-CoV-2 antibody in combination with a SARS-CoV-2 antigen, a fragment thereof, a variant thereof. The vaccine can be used to protect against any number of strains of SARS-CoV-2, thereby treating, preventing, and/or protecting against SARS-CoV-2 infection or associated pathologies, including COVID-19. The vaccine can significantly induce an immune response of a subject administered the vaccine, thereby protecting against and treating SARS-CoV-2 infection or associated pathologies, including COVID-19.

The vaccine can be a DNA vaccine, a peptide vaccine, or a combination DNA and peptide vaccine. The DNA vaccine can include a nucleic acid sequence encoding the SARS-CoV-2 antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the SARS-CoV-2 antigen by a peptide bond. The peptide vaccine can include a SARS-CoV-2 antigenic peptide, a SARS-CoV-2 antigenic protein, a variant thereof, a fragment thereof, or a combination thereof. The combination DNA and peptide vaccine can include the above described nucleic acid sequence encoding the SARS-CoV-2 antigen and the SARS-CoV-2 antigenic peptide or protein, in which the SARS-CoV-2 antigenic peptide or protein and the encoded SARS-CoV-2 antigen have the same amino acid sequence.

In some embodiments, the vaccine can induce a humoral immune response in the subject administered the vaccine. The induced humoral immune response can be specific for the SARS-CoV-2 antigen. The induced humoral immune response can be reactive with the SARS-CoV-2 antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the SARS-CoV-2 antigen. The neutralizing antibodies can be reactive with the SARS-CoV-2 antigen. The neutralizing antibodies can provide protection against and/or treatment of COVID-19 infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the SARS-CoV-2 antigen. These IgG antibodies can be reactive with the SARS-CoV-2 antigen. Preferably, the humoral response is cross-reactive against two or more strains of the COVID-19. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the SARS-CoV-2 antigen. The induced cellular immune response can be reactive to the SARS-CoV-2 antigen. Preferably, the cellular response is cross-reactive against two or more strains of the COVID-19. The induced cellular immune response can include eliciting a CD8⁺ T cell response. The elicited CD8⁺ T cell response can be reactive with the SARS-CoV-2 antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8+ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8⁺IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the SARS-CoV-2 antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4⁺IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ⁺ TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly.

SARS-CoV-2 Antigen

As described above, the vaccine comprises a SARS-CoV-2 antigen, a fragment thereof, a variant thereof, a nucleic acid molecule encoding the same, or a combination thereof. Coronaviruses, including SARS-CoV-2, are encapsulated by a membrane and have a type 1 membrane glycoprotein known as spike (S) protein, which forms protruding spikes on the surface of the coronavirus. The spike protein facilitates binding of the coronavirus to proteins located on the surface of a cell, for example, the metalloprotease amino peptidase N, and mediates cell-viral membrane fusion. In particular, the spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion. Accordingly, the SARS-CoV-2 antigen can comprise a SARS-CoV-2 spike protein, a S1 subunit of a SARS-CoV-2 spike protein, or a S2 subunit of a SARS-CoV-2 spike protein.

Upon binding cell surface proteins and membrane fusion, the coronavirus enters the cell and its singled-stranded RNA genome is released into the cytoplasm of the infected cell. The singled-stranded RNA genome is a positive strand and thus, can be translated into a RNA polymerase, which produces additional viral RNAs that are minus strands. Accordingly, the SARS-CoV-2 antigen can also be a SARS-CoV-2 RNA polymerase.

The viral minus RNA strands are transcribed into smaller, subgenomic positive RNA strands, which are used to translate other viral proteins, for example, nucleocapsid (N) protein, envelope (E) protein, and matrix (M) protein. Accordingly, the SARS-CoV-2 antigen can comprise a SARS-CoV-2 nucleocapsid protein, a SARS-CoV-2 envelope protein, or a SARS-CoV-2 matrix protein.

The viral minus RNA strands can also be used to replicate the viral genome, which is bound by nucleocapsid protein. Matrix protein, along with spike protein, is integrated into the endoplasmic reticulum of the infected cell. Together, the nucleocapsid protein bound to the viral genome and the membrane-embedded matrix and spike proteins are budded into the lumen of the endoplasmic reticulum, thereby encasing the viral genome in a membrane. The viral progeny are then transported by golgi vesicles to the cell membrane of the infected cell and released into the extracellular space by endocytosis.

In some embodiments, the SARS-CoV-2 antigen can be a SARS-CoV-2 spike protein, a SARS-CoV-2 RNA polymerase, a SARS-CoV-2 nucleocapsid protein, a SARS-CoV-2 envelope protein, a SARS-CoV-2 matrix protein, a fragment thereof, a variant thereof, or a combination thereof. The SARS-CoV-2 antigen can be a consensus antigen derived from two or more SARS-CoV-2 spike antigens, two or more SARS-CoV-2 RNA polymerases, two or more SARS-CoV-2 nucleocapsid proteins, two or more envelope proteins, two or more matrix proteins, or a combination thereof. The SARS-CoV-2 consensus antigen can be modified for improved expression. Modification can include codon optimization, RNA optimization, addition of a kozak sequence for increased translation initiation, and/or the addition of an immunoglobulin leader sequence to increase the immunogenicity of the SARS-CoV-2 antigen. In some embodiments the SARS-CoV-2 antigen includes an IgE leader, which can be the amino acid sequence set forth in SEQ ID NO:141.

SARS-CoV-2 Spike Antigen

The SARS-CoV-2 antigen can be a SARS-CoV-2 spike antigen, a fragment thereof, a variant thereof, or a combination thereof. The SARS-CoV-2 spike antigen is capable of eliciting an immune response in a mammal against one or more SARS-CoV-2 strains. The SARS-CoV-2 spike antigen can comprise an epitope(s) that makes it particularly effective as an immunogen against which an anti-SARS-CoV-2 immune response can be induced.

The SARS-CoV-2 spike antigen can be a consensus sequence derived from two or more strains of SARS-CoV-2. The SARS-CoV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the SARS-CoV-2 spike antigen. The SARS-CoV-2 spike antigen can comprise a signal peptide such as an immunoglobulin signal peptide, for example, but not limited to, an immunoglobulin E (IgE) or immunoglobulin (IgG) signal peptide. In some embodiments, the SARS-CoV-2 spike antigen can comprise a hemagglutinin (HA) tag. The SARS-CoV-2 spike antigen can be designed to elicit stronger and broader cellular and/or humoral immune responses than a corresponding codon optimized spike antigen.

The SARS-CoV-2 consensus spike antigen can be the amino acid sequence SEQ ID NO:134. In some embodiments, the SARS-CoV-2 consensus spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:134.

The SARS-CoV-2 consensus spike antigen can be the nucleic acid sequence SEQ ID NO:133, which encodes SEQ ID NO:134. In some embodiments, the SARS-CoV-2 consensus spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:133. In other embodiments, the SARS-CoV-2 consensus spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:134.

The SARS-CoV-2 consensus spike antigen can be operably linked to an IgE leader sequence. The SARS-CoV-2 consensus spike antigen operably linked to an IgE leader sequence can be the amino acid sequence SEQ ID NO:136. In some embodiments, the SARS-CoV-2 consensus spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:136.

The SARS-CoV-2 consensus spike antigen can be the nucleic acid sequence SEQ ID NO:135, which encodes SEQ ID NO:136. In some embodiments, the SARS-CoV-2 consensus spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:135. In other embodiments, the SARS-CoV-2 consensus spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:136.

Immunogenic fragments of SEQ ID NO:134 or SEQ ID NO:136 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:134 or SEQ ID NO:136. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:134 or SEQ ID NO:136 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:134 or SEQ ID NO:136. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:133 or SEQ ID NO:135. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:133 or SEQ ID NO:135. Immunogenic fragments can be at least 95%, at least 96%, at least 97% at least 98% or at least 99% homologous to fragments of SEQ ID NO:133 or SEQ ID NO:135. In some embodiments, immunogenic fragments include sequences that encode a leader sequence, such as for example an immunoglobulin leader, such as the IgE leader. In some embodiments, fragments are free of coding sequences that encode a leader sequence.

Outlier SARS-CoV-2 Spike Antigen

The SARS-CoV-2 antigen can be an outlier SARS-CoV-2 spike antigen, a fragment thereof, a variant thereof, or a combination thereof. The outlier SARS-CoV-2 spike antigen is capable of eliciting an immune response in a mammal against one or more SARS-CoV-2 strains. The outlier SARS-CoV-2 spike antigen can comprise an epitope(s) that makes it particularly effective as an immunogen against which an anti-SARS-CoV-2 immune response can be induced.

The outlier SARS-CoV-2 spike antigen can be a consensus sequence derived from two or more strains of SARS-CoV-2. The outlier SARS-CoV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the outlier SARS-CoV-2 spike antigen. The outlier SARS-CoV-2 spike antigen can comprise a signal peptide such as an immunoglobulin signal peptide, for example, but not limited to, an immunoglobulin E (IgE) or immunoglobulin (IgG) signal peptide. The outlier SARS-CoV-2 spike antigen can be designed to elicit stronger and broader cellular and/or humoral immune responses than a corresponding spike antigen.

The outlier SARS-CoV-2 spike antigen can be the amino acid sequence SEQ ID NO:138. In some embodiments, the outlier SARS-CoV-2 spike antigen can be the amino acid sequence having at least about 90%, 91%, 92%, 93%, 94%, 95%, 96%, 97%, 98%, 99%, or 100% identity over an entire length of the amino acid sequence set forth in SEQ ID NO:138.

The outlier SARS-CoV-2 spike antigen can be the nucleic acid sequence SEQ ID NO:137, which encodes SEQ ID NO:138. In some embodiments, the outlier SARS-CoV-2 spike antigen can be the nucleic acid sequence having at least about 90%, 91%, 92%, 93%, 94%, 95%, 96%, 97%, 98%, 99% or 100% identity over an entire length of the nucleic acid sequence set forth in SEQ ID NO:137. In other embodiments, the outlier SARS-CoV-2 spike antigen can be the nucleic acid sequence that encodes the amino acid sequence having at least about 90%, 91%, 92%, 93%, 94%, 95%, 96%, 97%, 98%, 99%, or 100% identity over an entire length of the amino acid sequence set forth in SEQ ID NO:138.

The outlier SARS-CoV-2 spike antigen can be operably linked to an IgE leader sequence. The outlier SARS-CoV-2 spike antigen operably linked to an IgE leader sequence can be the amino acid sequence SEQ ID NO:140. In some embodiments, the outlier SARS-CoV-2 spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:140.

The outlier SARS-CoV-2 spike antigen can be the nucleic acid sequence SEQ ID NO:139, which encodes SEQ ID NO:140. In some embodiments, the outlier SARS-CoV-2 spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:139. In other embodiments, the outlier SARS-CoV-2 spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:140.

Immunogenic fragments of SEQ ID NO:138 or SEQ ID NO:140 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:138 or SEQ ID NO:140. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:138 or SEQ ID NO:140 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:138 or SEQ ID NO:140. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:137 or SEQ ID NO:139. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:137 or SEQ ID NO:139. Immunogenic fragments can be at least 95%, at least 96%, at least 97% at least 98% or at least 99% homologous to fragments of SEQ ID NO:137 or SEQ ID NO:139. In some embodiments, immunogenic fragments include sequences that encode a leader sequence, such as for example an immunoglobulin leader, such as the IgE leader. In some embodiments, fragments are free of coding sequences that encode a leader sequence.

### 12. USE IN COMBINATION

The present invention also provides a method of treating, protecting against, and/or preventing disease in a subject in need thereof by administering a combination of the synthetic antibody and a therapeutic agent. In one embodiment, the therapeutic agent is an antiviral agent. In one embodiment, the therapeutic is an antibiotic agent. In one embodiment, the therapeutic agent is a SARS-CoV-2 vaccine. In one embodiment, the therapeutic agent is a small-molecule drug or biologic.

The synthetic antibody and a therapeutic agent may be administered using any suitable method such that a combination of the synthetic antibody and therapeutic agent are both present in the subject. In one embodiment, the method may comprise administration of a first composition comprising a synthetic antibody of the invention by any of the methods described in detail above and administration of a second composition comprising a therapeutic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising a therapeutic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising a therapeutic agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the therapeutic agent. In one embodiment, the method may comprise administration of a first composition comprising a therapeutic agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the therapeutic agent. In one embodiment, the method may comprise administration of a first composition comprising a synthetic antibody of the invention by any of the methods described in detail above and a second composition comprising a therapeutic agent concurrently. In one embodiment, the method may comprise administration of a first composition comprising a synthetic antibody of the invention by any of the methods described in detail above and a second composition comprising a therapeutic agent concurrently. In one embodiment, the method may comprise administration of a single composition comprising a synthetic antibody of the invention and a therapeutic agent.

Non-limiting examples of antibiotics that can be used in combination with the synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

### 13. GENERATION OF SYNTHETIC ANTIBODIES IN VITRO AND EX VIVO

In one embodiment, the synthetic antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The present invention has multiple aspects, illustrated by the following non-limiting examples.

### 14. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: In Vivo Production of Monoclonal Antibodies Against SARS-CoV-2/COVID-19 Using Synthetic DNA

CR3022 is a cross-reactive monoclonal Ab (mAb) that binds SARS-CoV-2. CR3022 binds an epitope on the SARS-CoV Spike (S) protein within the receptor-binding domain (RBD).

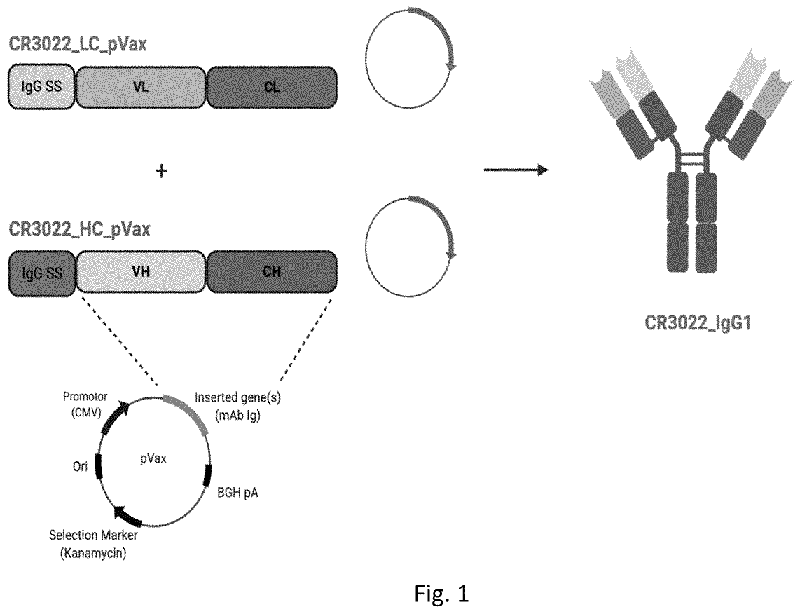

An optimized DNA-encoded monoclonal antibody (dMABs) was developed against SARS-CoV-2 virus for prevention, treatment and/or diagnostic use, including treatment or prevention of a SARS-CoV-2-mediated disease (e.g., COVID-19). Novel engineering approaches were used to develop a dMAB for in vivo delivery of anti-SARS-CoV-2/COVID-19 mAbs with enhanced production (expression, processing, assembly, secretion): 1) Ig gene (heavy chain/HC and light chain/LC) sequences were optimized at the nucleic acid level (DNA and RNA), 2) an optimized IgG leader sequence was placed ahead of genes to ensure secretion into circulation, and 3) a modified pVax expression vector which contains various components to ensure transcript processing was used (FIG. 1).

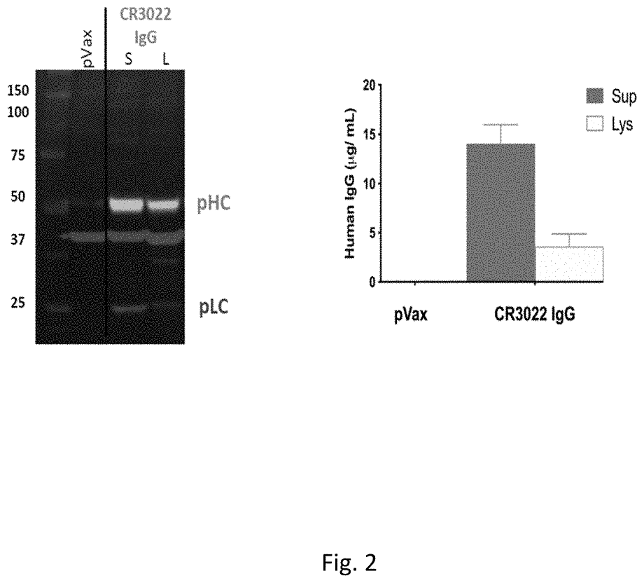

Co-transfection of CR3022 LC and HC plasmids in vitro resulted in production and secretion of CR3022 IgG1 antibody, which was detected in culture supernatant western blot and quantified by ELISA (FIG. 2).

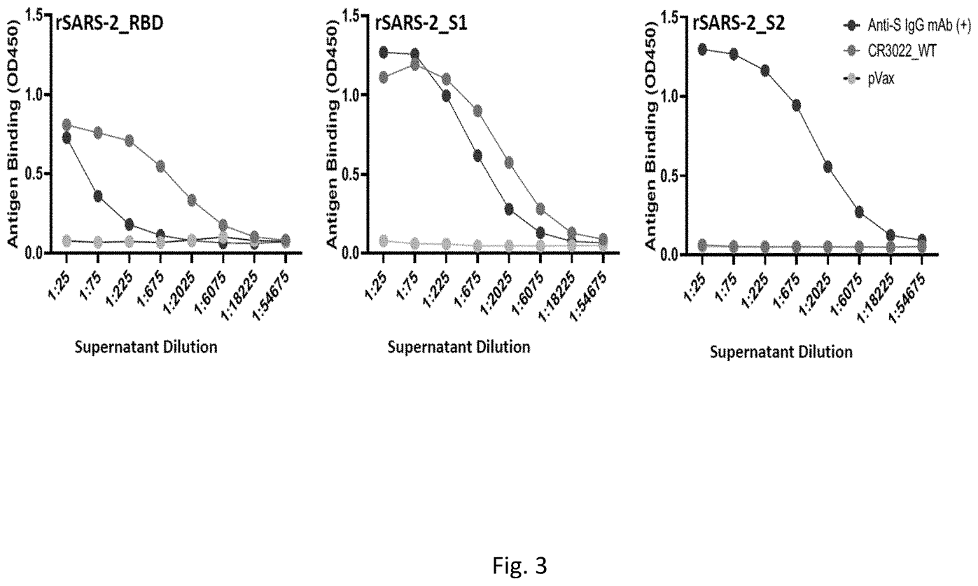

In vitro-expressed CR3022 dMAB demonstrated binding to appropriate domains of the SARS-CoV-2 Spike (S) protein (FIG. 3). Consistent with previous reports, it binds recombinant versions of the receptor binding domain (RBD). The RBD is located within the larger Subunit 1 (S1) domain of the S protein, therefore, the CR3022 dMAB also bound recombinant S1 (FIG. 3).

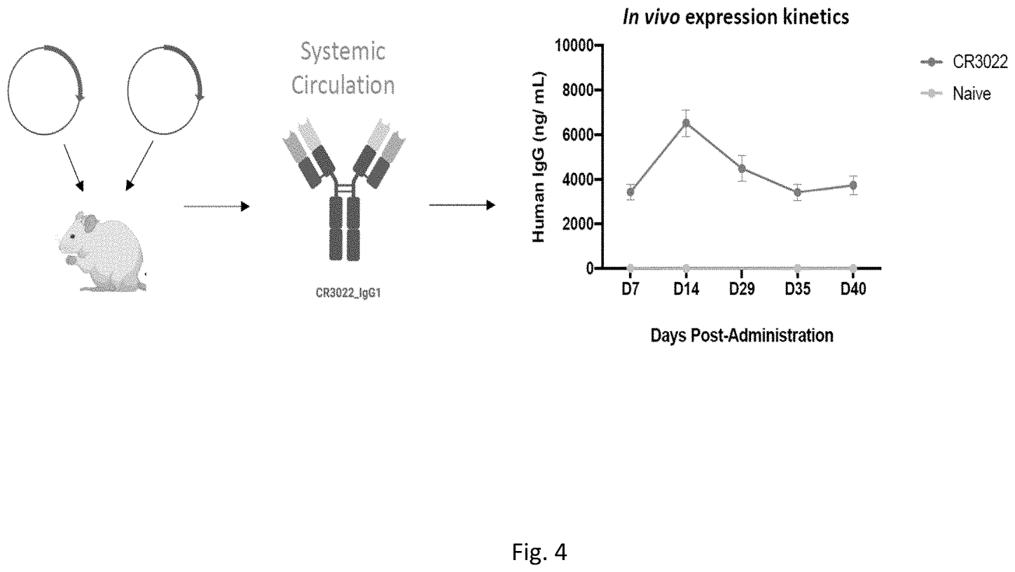

An analysis of the in-vivo expression kinetics showed consistent and durable expression of the human CR3022 IgG1 dMAB in mice following dual-plasmid administration/electroporation (FIG. 4).

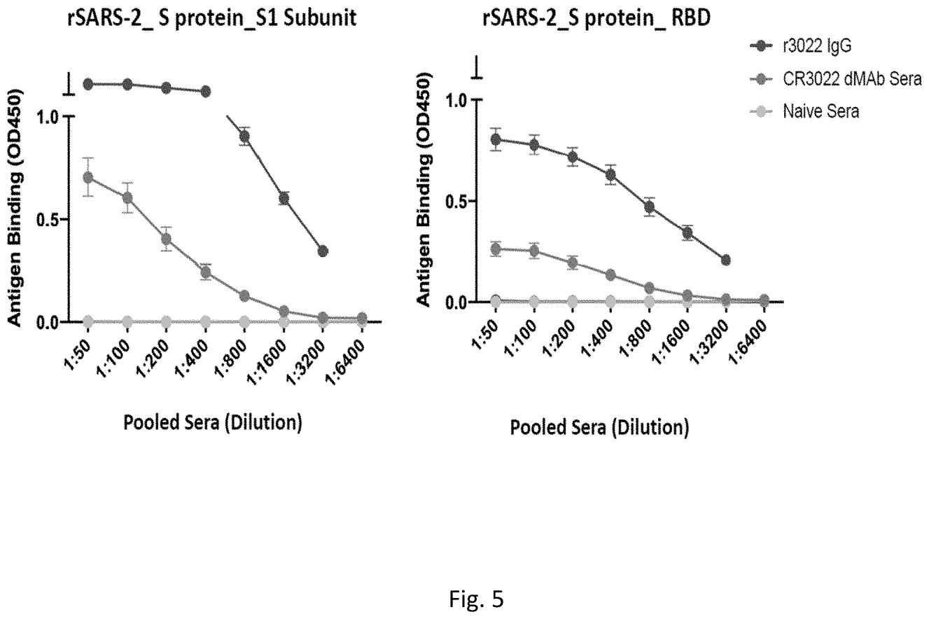

Sera from mice administered the CR3022 dMAB via electroporation demonstrated binding to recombinant SARS-CoV-2 RBD and S1 domains via ELISA (FIG. 5).

### Example 2: In Vivo Production of Monoclonal Antibodies Against SARS-CoV-2/COVID-19 Using Synthetic DNA

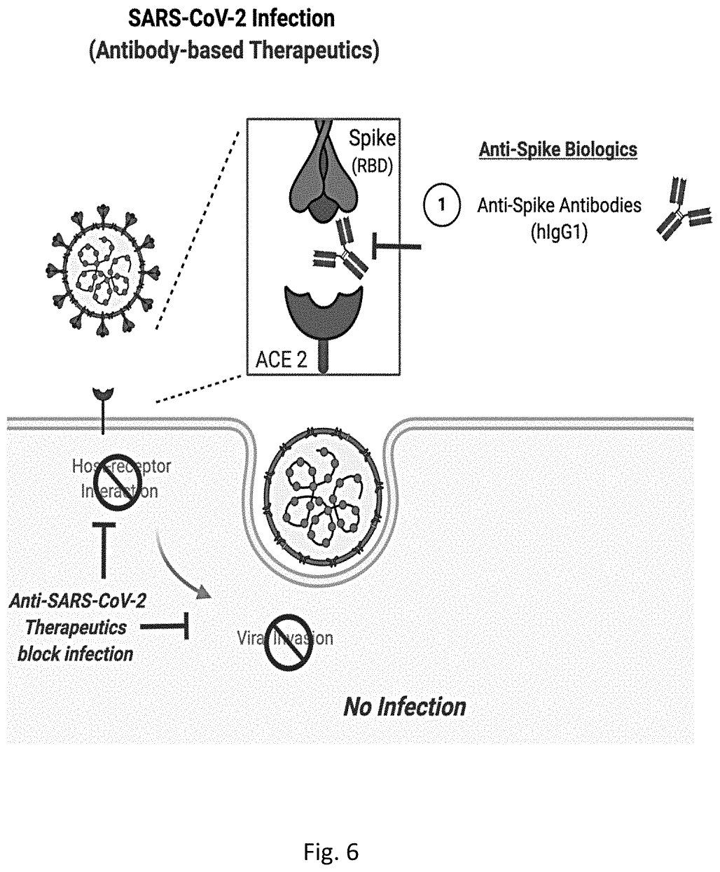

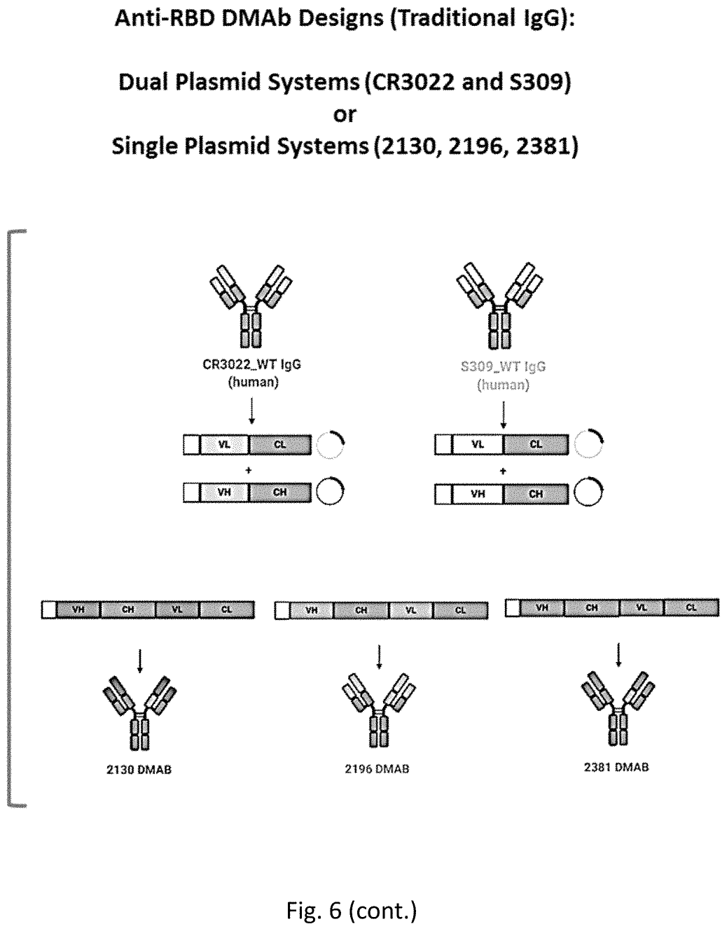

Optimized DNA-encoded monoclonal antibodies (dMABs) were developed against SARS-CoV-2 virus for prevention, treatment and/or diagnostic use (e.g., for treatment of COVID-19 disease). DMABs were developed for in vivo delivery of anti-SARS-CoV-2/COVID-19 mAbs with enhanced production (expression, processing, assembly, secretion) using a single plasmid or dual plasmid system (FIG. 6).

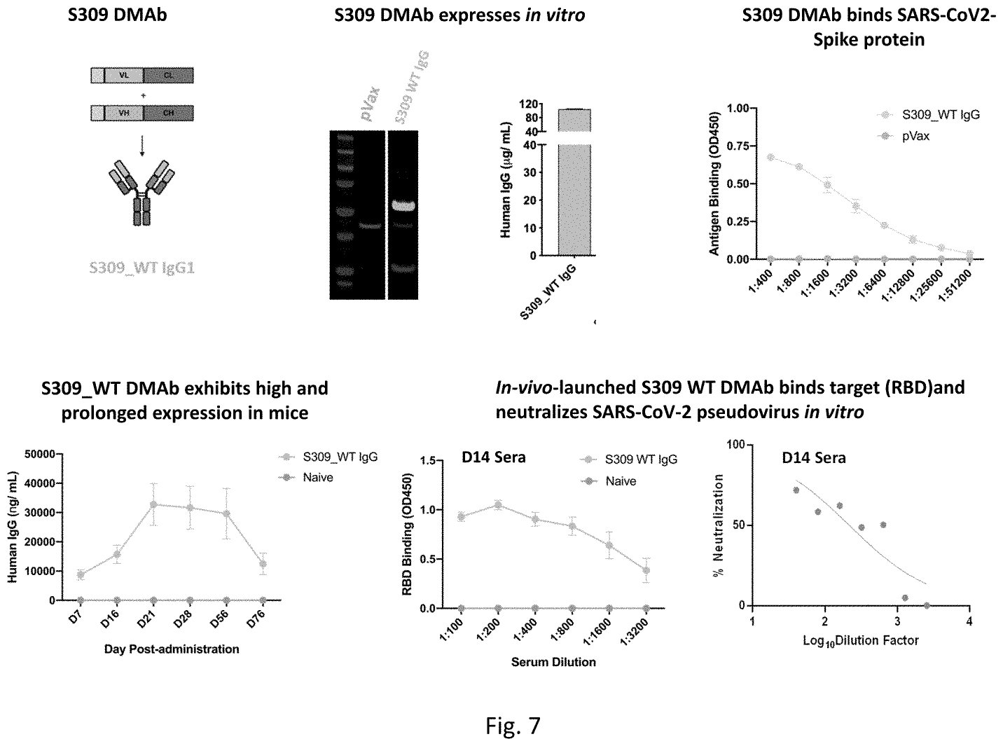

Co-transfection of S309 LC and HC plasmids in vitro resulted in production and secretion of neutralizing antibodies which bind to the SARS-CoV-2 spike protein receptor binding domain (RBD) (FIG. 7).

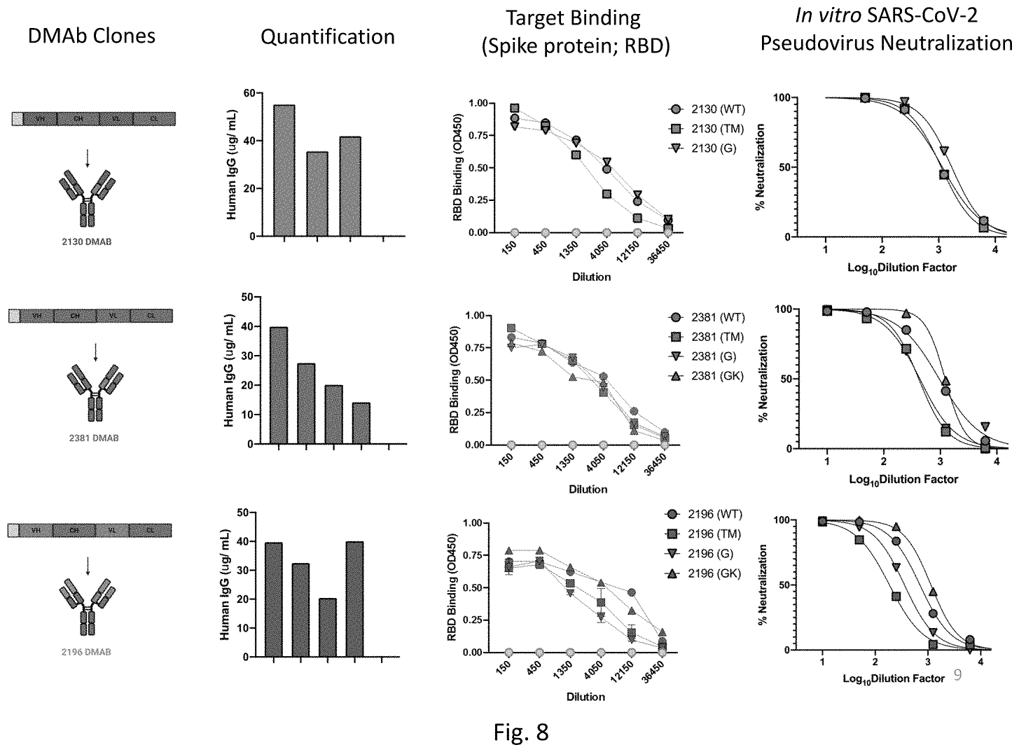

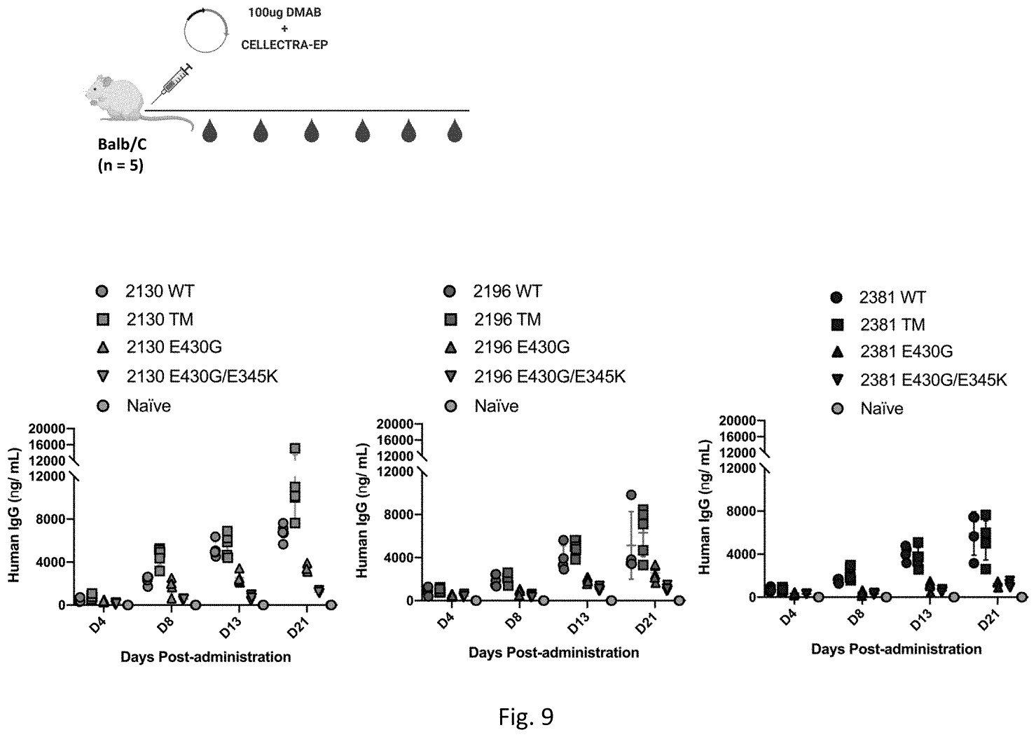

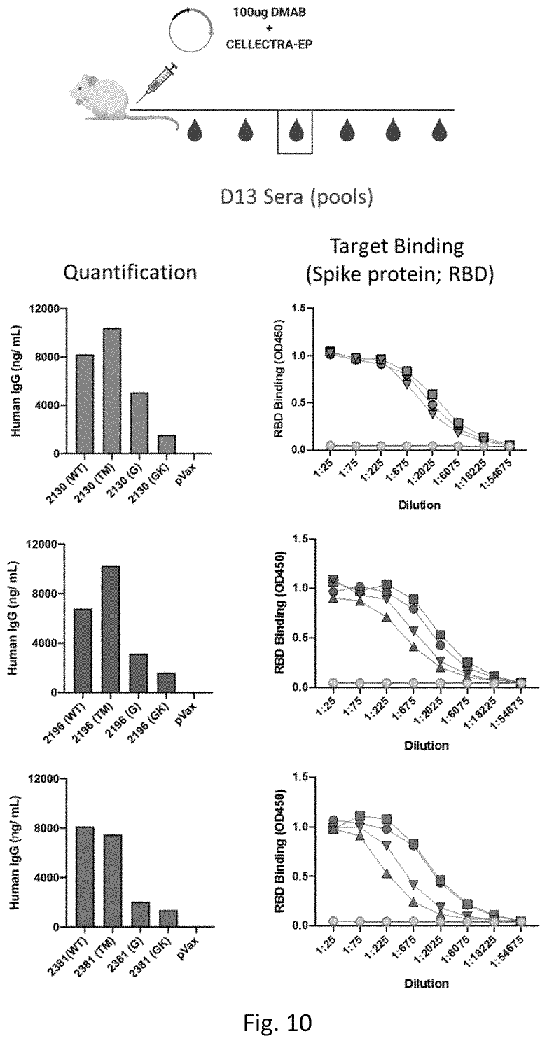

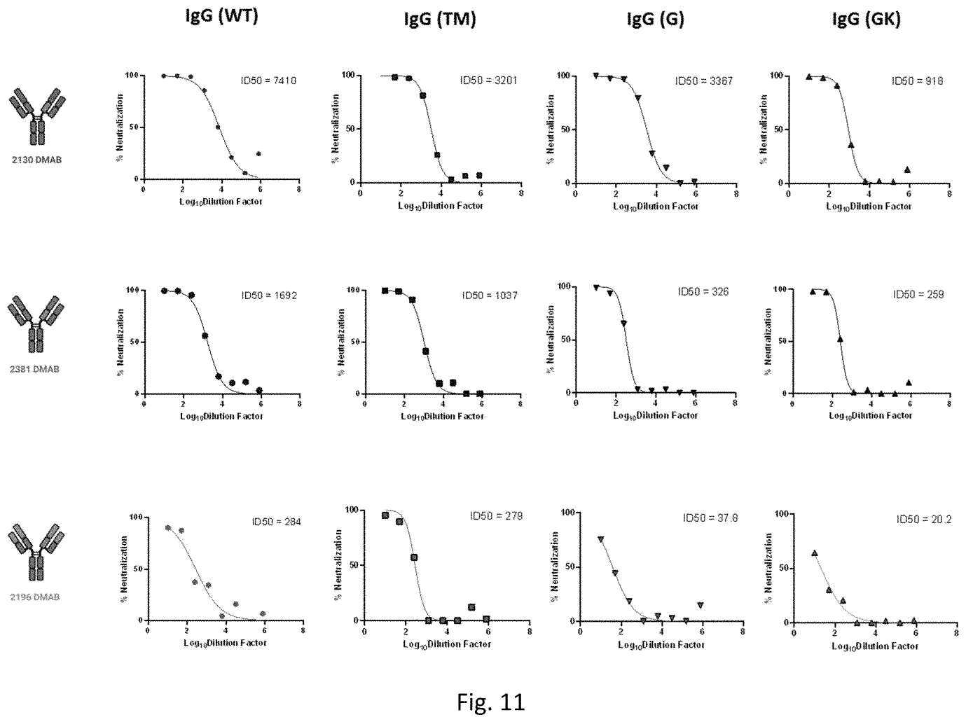

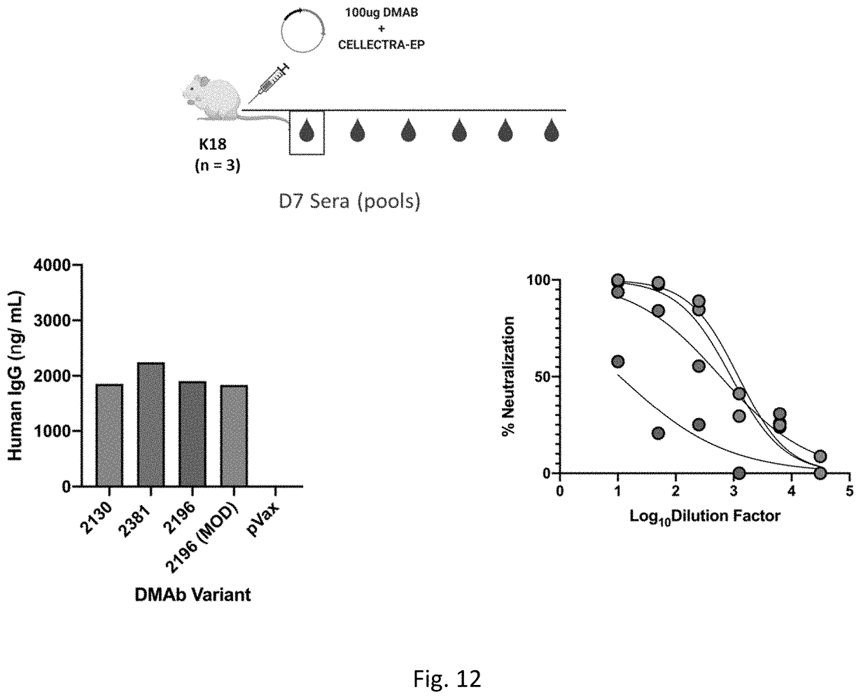

Transfection of 2130, 2381 and 2196 DMAb plasmids in vitro resulted in expression of the dMABs and binding to appropriate domains of the SARS-CoV-2 Spike (S) protein (FIG. 8 and FIG. 9). An analysis of the in-vivo expression kinetics showed consistent and durable expression of the 2130, 2381 and 2196 DMAbs following administration/electroporation (FIG. 10). Sera from mice administered the 2130, 2381 and 2196 DMAbs via electroporation demonstrated binding to recombinant SARS-CoV-2 RBD and S1 domains via ELISA (FIG. 11). Finally, a modified DMAb (2196_MOD DMAb) demonstrated in vivo expression and had more potent neutralization than the parental 2196 WT DMAb (FIG. 12).

### Example 3: Novel Monoclonal Antibodies Against the SARS-CoV-2 Spike Glycoprotein: Potential Utility as Therapeutic for COVID-19 Patients

The Spike protein of SARS-CoV-2 virus is reported to bind to its receptor ACE2. mAbs which target the vulnerable sites on viral surface proteins are increasingly known as a promising class of drugs against infectious diseases and have shown therapeutic efficacy for a number of viruses (Wang et al., 2020a). In line with this, identifying and cloning mAbs which can specifically target surface viral proteins to block the viral entry to host cells seems to be a highly attractive approach for the prevention and treatment of SARS-CoV-2 (Chen et al., 2020). The spike protein of SARS-CoV-2 undergoes major conformational alterations and exposes the RBD and important residues for receptor binding in order to engage the host cell receptor ACE2. Thus, the binding of RBD to ACE2 receptor protein leads to the detachment of S1 from S2, ultimately resulting in virus-host membrane fusion mediated by S2 and the entry of virus. Thus, the role of spike protein in the infection process of SARS-CoV-2 into host cells is highly critical and hence is a highly potent target for developing effective mAbs (Chen et al., 2020). Antibody responses against SARS-CoV-2 along with other enveloped viruses usually comprise of IgM, IgG and IgA antibodies to glycoproteins of the virus envelope and to nucleoproteins. IgG antibodies against the envelope proteins exhibit different functional features which enables them to provide the most effective systemic antibody response against the virus (French and Moodley, 2020).

Without being bound by theory, it was hypothesized that immunotherapeutic approaches including vaccines and antibodies may provide benefit to patients. In this regard, plasma, from patients recovered from disease, has been used to treat severely ill COVID-19 patients (Ni et al., 2020; Zhang et al., 2020). This has prompted the development of antibodies against spike protein for therapeutic use. In this study, 10 IgG mAbs specific to SARS-CoV-2-Spike protein were developed and successfully cloned. In order to be effective, the antibodies should meet several characteristics including specificity, high affinity binding to antigen and the ability to compete with the spike protein binding to receptor ACE2, thus blocking the infection of cells by the virus. All the 10 mAbs were found to have specific and strong binding to SARS-CoV-2-RBD. Further they also caused blocking of the interaction between SARS-CoV-2-RBD and ACE2 receptor. Further, the glycomic profile of the antibodies (especially WCoVA7) suggested them to have high Fc-mediated effector functions. Therefore, Fc-mediated effector functions of these antibodies need to be examined further. Further they also caused blocking of the interaction between SARS-CoV-2-RBD and ACE2 receptor. In addition, all the 10 IgG clones exhibited efficient neutralization of SARS-CoV-2 protein pseudotyped virus infection with the lowest IC50 value obtained at 2.6 ng/ml and majority with less than 10 ng/ml. Further, in this study it is shown that these IgG clones efficiently neutralized SARS-CoV-2-D614G mutated virus, with 4 of them exhibiting IC50 value less than 10 ng/ml. Significantly low IC50 values in terms of neutralization efficacy strongly indicates the high potential of these Spike antibodies against SARS-CoV-2. Notably, a number of different naturally occurring variants in the SARS-CoV-2-Spike protein have been reported. Initial findings implied that increased fatality rate due to COVID-19 may be linked with D614G, the most dominant variant. This mutation might supposedly result in conformational alteration in the Spike protein, ultimately leading to enhanced infectivity (Li et al., 2020). Without being bound by theory, it is hypothesized that this varied degree of infectivity and transmissibility might attribute to the different reactivity observed with the SARS-CoV-2-Spike neutralization antibodies between the wild type and the D614G mutated SARS-CoV-2 virus in this study. Altogether, these anti-SARS-CoV-2-Spike-ACE2 blocking mAbs hold significant potential and thus can be explored further as specific therapeutic option against this severe worldwide pandemic.

The results presented here underscore the potential utility of passive immunotherapy for the treatment of COVID-19. The identification and characterization of monoclonal antibodies against the Spike protein of SARS-CoV-2 add a valuable set of agents with therapeutic potential. Of the 10 mAbs described, all of them exhibit specificity and high affinity towards RBD of spike. Hence, monoclonal antibodies have advantages over the use of convalescent polyclonal sera from patients recovering from COVID-19. While the mAbs are independent from one another, the epitope recognized by the antibodies is not clear. The functional characteristics of the antibodies show their ability to neutralize SARS-CoV-2 Spike protein pseudotyped virus which is a surrogate measure of their effect on the virus. The antibodies should be evaluated in the infection studies with SARS-CoV-2 virus. The development of unique and specific mAbs against the Spike antigen and epitopes is warranted as they would enable the use of a “cocktail” (mixture of specific biologically active mAbs) to simultaneously engage multiple neutralizing epitopes on the virions for enhanced therapeutic potency. Further, the development of biologically active anti-SARS CoV-2 neutralizing mAbs may also provide information about the epitopes within the antigen as the likely targets to develop an active vaccine for prophylactic purposes. In addition, the highly potent SARS-CoV-2-Spike mAbs possess potential to serve as candidate biologics from both prophylactic and therapeutic standpoints as well as tool for the development of SARS-CoV-2 specific diagnostic assays of high clinical significance.

The materials and methods used are now described

Cell Culture

HEK293T cells were obtained from the ATCC and CHO-ACE2 cell line (stably expresses ACE2 on the cell surface) was procured from the Creative Biolabs, USA. Both the cell types were maintained in D10 media comprised of Dulbecco's Modified Eagle Medium (Invitrogen Life Science Technologies, USA) with heat-inactivated fetal calf serum (10%), glutamine (3 mM), penicillin (100 U/ml), and streptomycin (100 U/ml). R10 media comprising of RPMI1640 (Invitrogen Life Science Technologies, USA), heat-inactivated fetal calf serum (10%), glutamine (3 mM), penicillin (100 U/ml), and streptomycin (100 U/ml) was used for the mouse splenocyte cells. All the cells were maintained at 37° C. and 5% CO₂.

Construction of SARS-CoV-2-Spike Synthetic DNA and Protein

The SARS-CoV-2-Spike plasmid DNA encoded construct was determined by the alignment of RBD protein sequences available in the PubMed database. The sequences corresponding to the immunogen insert was genetically optimized for enhanced expression in human and an IgE leader sequence was added to facilitate expression (Muthumani et al., 2016). The synthetic vaccine construct was designed and was provided to a commercial vendor (Genscript, NJ, USA) for synthesis. The insert was then subcloned into a modified pMV101 expression vector under the control of the cytomegalovirus immediate-early promoter as described earlier (Muthumani et al., 2015; Muthumani et al., 2016).

Generation and Evaluation of Anti-SARS-CoV-2 Hybridomas

Balb/c mice were immunized with synthetic, consensus sequence of SARS-CoV-2-Spike antigens on day 0 and 14 at 50 μg per mouse by intramuscular route of immunization followed by subcutaneous delivery of recombinant Spike-RBD protein (100 μg/mouse). Sera from the immunized mice were collected and evaluated by ELISA to detect the presence of antibodies targeting the SARS-CoV-2 Spike. After confirmation, mouse splenocytes were used to generate hybridomas as described previously (Choi et al., 2020). Subsequently, positive hybridoma clones were characterized by an indirect ELISA and those selected were further subcloned and expanded. The antibodies were purified from hybridoma supernatants and used for further studies.

Enzyme-Linked Immunosorbent Assay (ELISA)

ELISA assays were performed with the mAb clones in order to measure target antigen binding. For measuring total IgG in hybridoma supernatants, MaxiSorp high binding 96 well ELISA plates (ThermoFisher, USA) were coated with 1 μg/mL recombinant SARS-CoV-2-RBD (Sino Biological, USA) as well as full length-Spike overnight at 4° C. Following blocking with 10% FBS in PBS for an hour, the plates were incubated with serially diluted mAb clones using PBS with 1% FBS for 2 hours. Then the samples were probed with anti-mouse IgG antibodies conjugated to horseradish peroxidase (HRP) (Sigma Aldrich, USA) at 1:20,000 dilution for 1 hour. Following this, tetramethylbenzidine (TMB) substrate (Sigma Aldrich, USA) was added to all the wells and incubated for 10 minutes. 2N H₂SO₄ was then used to stop the reaction. Finally, the optical density was measured at 450 nm with the help of an ELISA plate reader (Biotek, USA). Further, SARS-CoV-2-RBD and full-length Spike specific antibody mean endpoint titers for all the mAb clones were also determined. The titer is determined at the highest dilution with S/N (Signal/Negative) ratio ≥2.1. The signal was designated as positive binding to SARS-CoV-2-RBD or full-length Spike compared to the negative control which was binding of an irrelevant unrelated mAb to the antigen. The OD450 in the negative control is the average of two technical replicates and was designated as the antibody endpoint titer for the IgG mAb clones.

Western Blot Analysis

A binding Western blot analysis was performed to evaluate anti-CoV-2 mAb-binding specificity. Briefly, 5 μg of in-house generated recombinant human SASR-CoV-2-full length Spike and 2.5 μg of SARS-CoV-2-RBD (Sino Biological Inc, USA) proteins were run in 12% NUPAGE Novex polyacrylamide gels (Invitrogen Life Science Technologies, USA) and transferred to PVDF membranes (Invitrogen Life Science Technologies, USA). The membranes were blocked using Odyssey blocking buffer (LiCor BioSciences, USA) and then incubated with the supernatants from mAb clones for overnight at 4° C. After incubation, the membranes were washed with PBS containing 0.05% Tween 20 or PBST. Subsequently, the membranes were stained with IRDye800 goat anti-mouse secondary antibody (LI-COR Biosciences, USA) at RT and then again washed with PBST. Finally, the membranes were scanned using a LI-COR Odyssey CLx imager. Further, for determining the heavy and light chain expressions of the mAb clones, this assay was performed, in which 6 ng of each mAb clones was run in 12% NuPAGE Novex polyacrylamide gels (Invitrogen Life Science Technologies, USA) and subsequently probed with Goat anti-mouse IgG secondary antibody (LiCor BioSciences, USA).

SPR (Surface Plasmon Resonance) Analysis of SARS-CoV-2-Spike Monoclonal Antibody Clones' Binding to RBD Protein

Binding of mAb clones to SARS-CoV-2-Spike protein was measured using a Biacore T200 SPR system. SARS-CoV-2-RBD (Sino Biological Inc, USA) protein was immobilized with running buffer of 10 mM HEPES, pH 7.4, 150 mM NaCl, 0.05% Tween20 using standard amine coupling procedures to a carboxymethyl dextran sensor chip (CMD200L, Xantec Bioanalytics). Briefly, the chip was first washed with 0.1 M sodium borate, pH 9.0, 1M NaCl, followed by activation with EDC/NHS for 8 min using a running buffer of MilliQ distilled water. After activation, 10 μg/mL of each protein in 10 mM sodium acetate, pH 5 until the desired immobilization level was achieved. Approximately 2500 RU SARS-CoV-RBD protein was immobilized on the flow cell. 5000 RU of bovine serum albumin (BSA) was also immobilized to another flow cell which served as a negative control. After immobilization, the remaining activated sites were blocked with 1 M ethanolamine, pH 8.5. The running buffer was then switched to 10 mM HEPES, pH 7.4, 150 mM NaCl, 0.05% Tween20. Each IgG mAb clones were tested at three different concentrations in duplicate (0.13 nM, 0.41 nM and 1.2 nM). Dilutions were prepared in running buffer. The association time was 300 s and the dissociation time was 600 s with a flow rate of 30 μL/min and a measurement temperature of 20° C. After each injection, the surface was regenerated by injecting 20 mM glycine, pH 2.0 for 60 seconds. Data were collected and analyzed using Biacore Evaluation Software. Further, kinetic parameters for SARS-CoV-2-RBD binding to IgG mAb clones were determined using a Protein A/G coated carboxymethyl dextran sensor chip (Xantec Bioanalytics, Germany) in a Biacore T200 SPR system. Approximately 400 RU of each mAb clone was captured on the chip surface for each concentration of antigen. SARS-CoV-2-RBD was tested at concentrations ranging from 0-100 nM and the flow rate was 30 μL/min. The reference surface was coated with 400 RU of mouse IgG isotype control. The association time was 210 seconds and the dissociation time was 900 seconds. After each concentration of antigen, the antibody-antigen complex was removed from the chip using 20 mM glycine pH 2.0. Data are the mean of duplicate determinations and kinetic parameters were determined using the 1:1 binding model in the Biacore T200 Evaluation software.

Glycan Analysis

500 μl of hybridoma supernatants were concentrated using Amicon Ultra-0⋅5 Centrifugal Filter Unit (Millipore Sigma). Bulk IgG from three C57BL/6 mice and human plasma sample (Innovative Research) were used as controls. Total IgG was purified using Pierce™ Protein G Spin Plate for IgG Screening (Thermo Fisher), and IgGs were further concentrated using Amicon Ultra-0⋅5 Centrifugal Filter Unit (Millipore Sigma). N-glycans were released using peptide-N-glycosidase F (PNGase F) and labelled with 8-aminopyrene-1,3,6-trisulfonic acid (APTS) using the GlycanAssure APTS Kit (Thermo Fisher), following the manufacturer's protocol. Labelled N-glycans were analysed using the 3500 Genetic Analyzer capillary electrophoresis system. Relative abundance of IgG glycan structures was quantified by calculating the area under the curve of each glycan structure divided by the total glycans.

SARS-CoV-2-Surrogate Virus Neutralization Assay

The SARS-CoV-2-surrogate virus neutralization test kit (Genscript, USA) was used for detecting the potential of the mAbs to neutralize SARS-CoV-2-RBD and ACE2 interaction. It is a species and isotype independent blocking ELISA detection tool which determines the circulating neutralizing antibodies against SARS-CoV-2 that can block protein-protein interaction between RBD and human ACE2 receptor. Briefly, 500 ng/ml of each mAb clones and controls were pre incubated with HRP conjugated RBD (HRP-RBD) for 30 minutes at 37° C. to facilitate binding between mAb clones and HRP-RBD. Subsequently, the mixture was added to the capture plate pre coated with human ACE2 receptor protein and incubated for 15 minutes at 37° C. Following washing of the plate using Wash solution (Genscript, USA), TMB substrate was added to all the wells and incubated for 15 minutes. Then, stop solution (Genscript, USA) was added to each well to quench the reaction. Finally, the absorbance was measured at 450 nm using an ELISA plate reader (Biotek, USA) and the inhibition values were determined. The cutoff value of 20 was considered based on a panel of confirmed COVID-19 patient sera and healthy control sera validated by Genscript, USA.

Flow Cytometry-Based Receptor Binding Inhibition Assay

The inhibition of the binding of SARS-CoV-2-spike protein to the ACE2 receptor was also evaluated through a flow cytometry-based assay using commercially available CHO-ACE2 cell line. For this assay, 2.5 ug/ml of S1+S2 ECD-his tagged (Sino Biological, USA) was incubated with each of the 10 IgG mAb clones (500 ng/ml) on ice for 60 min. The mixtures were then transferred to the already plated CHO-ACE2 cells (150,000 cells/well) and then again incubated on ice for 90 min. Subsequently, the cells were washed twice with PBS followed by staining with Surelight® APC conjugated anti-his antibody (Abcam, USA). Spike protein pre-incubated with recombinant human ACE2 (Invitrogen Life Science Technologies, USA) was used as positive control. All the data were acquired from an LSRII flow cytometer (BD Biosciences) and analyzed with the help of FlowJo software (Version 10; Tree Star, USA).

SARS-CoV-2 Pseudovirus Production and Neutralization Assays

The SARS-CoV-2 pseudovirus was produced by co-transfection of HEK293T cells with 1:1 ratio of DNA plasmid encoding SARS-CoV-2 Spike protein (GenScript, USA) and backbone plasmid pNL4-3.Luc.R-E-(NIH AIDS Reagent) using GeneJammer (Agilent, USA) in D10 medium. The supernatant containing pseudovirus was harvested 48 hours post-transfection and enriched with FBS to 12% total volume, steri filtered and stored at −80° C. The pseudovirus was titrated using the stable commercially available CHO-ACE2 cell line. For the neutralization assay, 10,000 CHO-ACE2 cells in 100 μL D10 media were plated in 96-well plates and rested overnight at 37° C. and 5% CO₂ prior to the neutralization assay. The following day, serially diluted samples were incubated with SARS-CoV-2 pseudovirus at room temperature for 90 minutes, before the mixture was added to the already plated CHO-ACE2 cells. The cells were incubated at 37° C. and 5% CO₂ for 72 hours, and subsequently harvested and lysed with BriteLite reagent (PerkinElmer, USA). Luminescence from the plates were recorded with a BioTek plate reader and used to compute percentage neutralization of the samples at each dilution.

Statistical Analysis

Statistical analyses were performed using Graph Pad Prism software by either a student's t-test or the nonparametric Spearman's correlation test for calculating the statistical significance. The data are represented as the mean±Standard Error of the Mean (SEM). p value <0.05 was considered significant for all the tests.

The experimental results are now described

Generation and Characterization of Antibodies Targeting SARS-CoV-2-Spike

Increasing lines of evidence suggest Coronavirus neutralizing antibodies (NAbs) to demonstrate high efficacy in treating a diverse range of infectious diseases owing to their highly specific antiviral activity and safety. Notably, SARS-CoV and MERS-CoV RBD-vaccine studies revealed strong polyclonal antibody responses in the in vivo setting, which caused inhibition of viral entry, implying the high potential of anti-Spike antibodies to inhibit the entry of SARS-CoV-2 coronavirus (Ju et al., 2020; Shi et al., 2020).

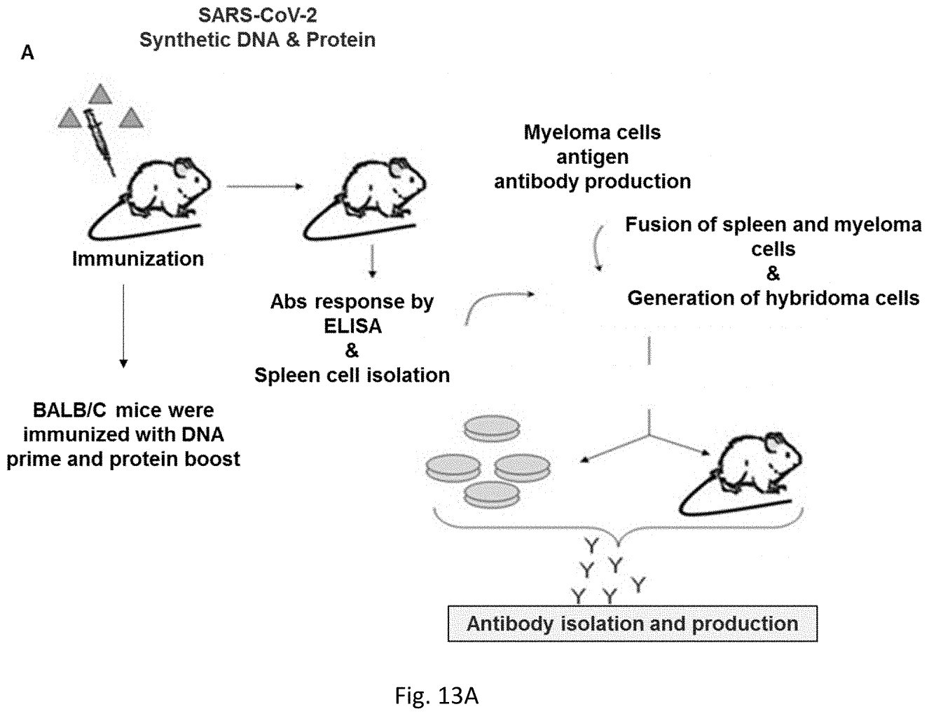

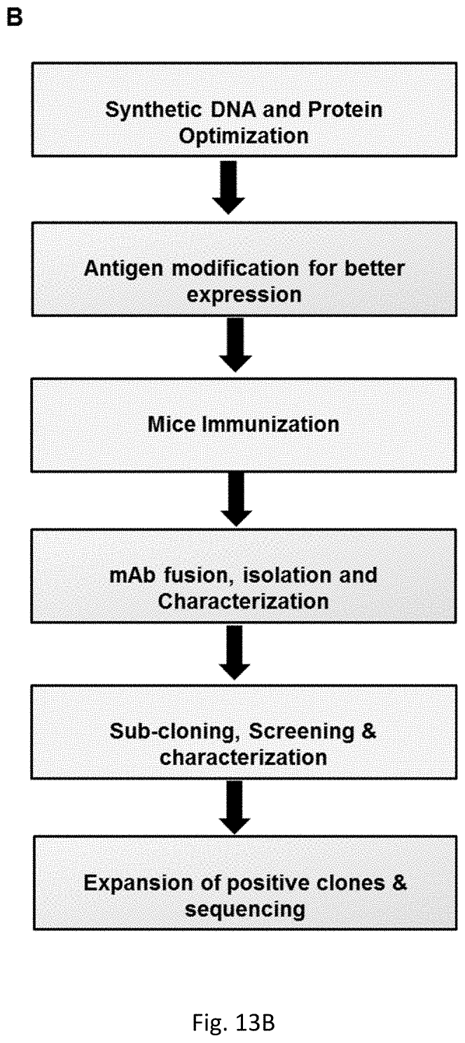

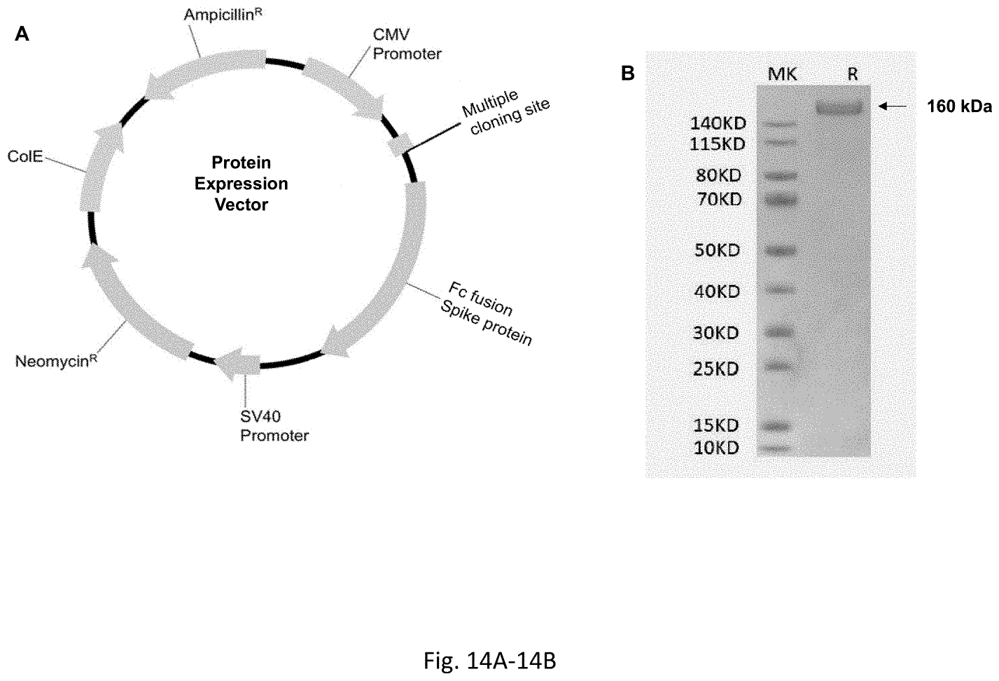

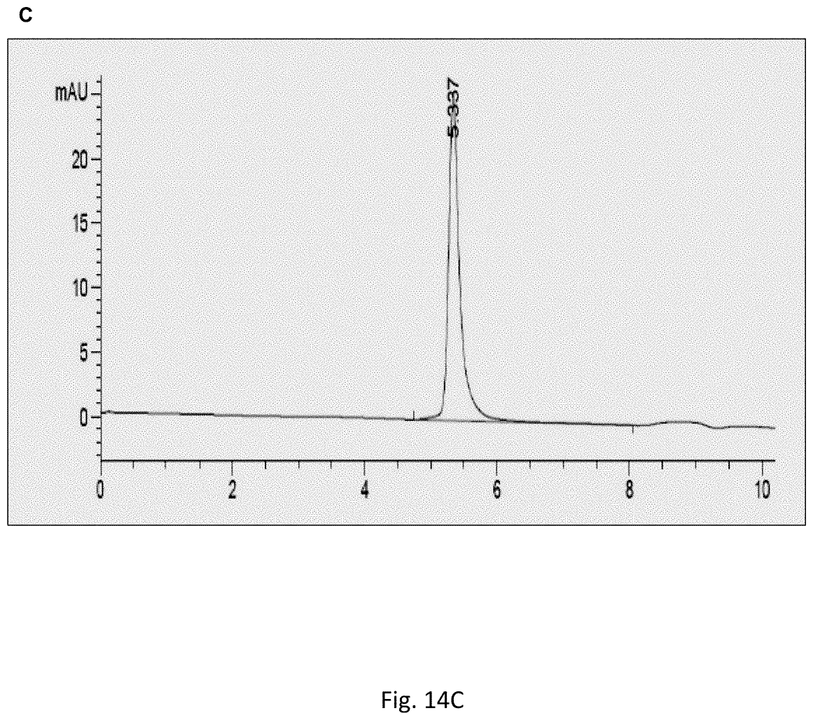

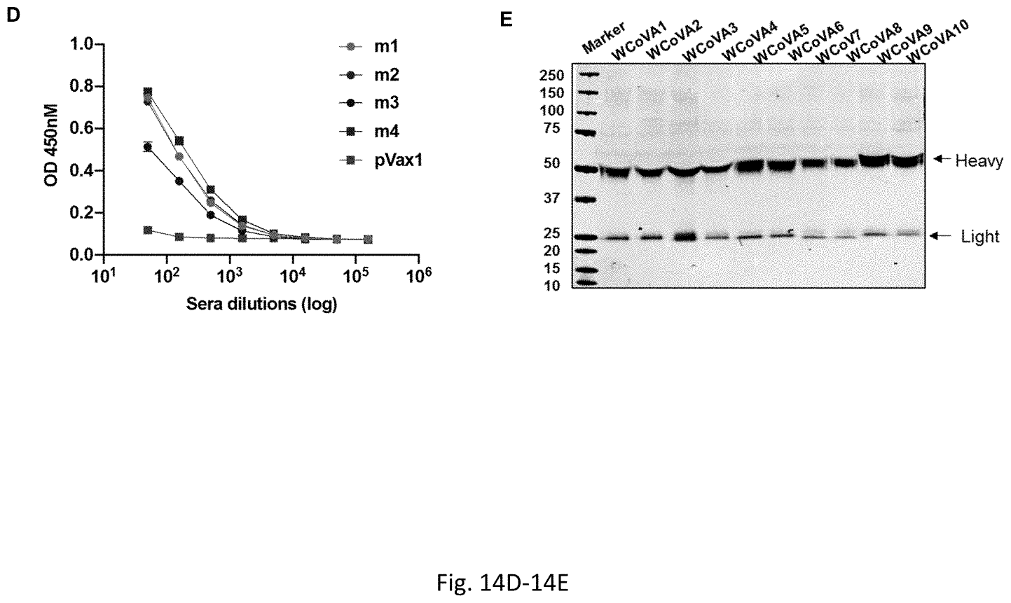

In this study, Balb/c mouse was used for immunization with synthetic DNA plasmid constructs encoding the consensus sequence of full-length SARS-CoV-2-Spike antigen (Smith et al., 2020) as prime and SARS-CoV-2-Spike-RBD recombinant protein as boost as described earlier (Choi et al., 2020). The strategy and the steps used for immunization and follow up procedures are outlined in FIG. 13A-B. Finally, B lymphocytes were isolated from the spleens of the immunized mouse and were used to generate hybridomas. For the analysis of sera from the immunized animal, full length spike protein expression construct was generated as shown in FIG. 14A. SARS-CoV-2 Spike full length protein was expressed using the mammalian cell system with a Fc tag and Avi-tag at the C-terminal and its size and purity were verified by SDS-PAGE and HPLC techniques (FIG. 14B-C). Full length spike protein (160 KDa) revealed a clear band at the correct position on SDS-PAGE gel. This protein was also found to be of high purity as suggested by the sharp single peak obtained from HPLC. Furthermore, the protein specificity was confirmed by ELISA using immune sera from mice vaccinated with SARS-CoV-2 Spike DNA (FIG. 14D). Above 800 hybridomas were generated, which were then screened in order to determine the clones with ability to produce antibodies with the highest affinity against SARS-CoV-2 Spike protein. This screening led to the identification of the best 10 IgG mAbs (WCoVA1, WCoVA2, WCoVA3, WCoVA4, WCoVA5, WCoVA6, WCoVA7 WCoVA8, WCoVA9 and WCoVA10). The isotype analysis of the mAbs showed WCoVA1, WCoVA2, WCoVA3, WCoVA4, WCoVA7 and WCoVA8 clones with IgG1, whereas WCoVA5, WCoVA6, WCoVA9 and WCoVA10 clones with IgG2a. The individual hybridoma clones were further evaluated for binding and specificity.

Binding and Specificity Analysis of SARS-CoV-2 Spike Monoclonal Antibodies

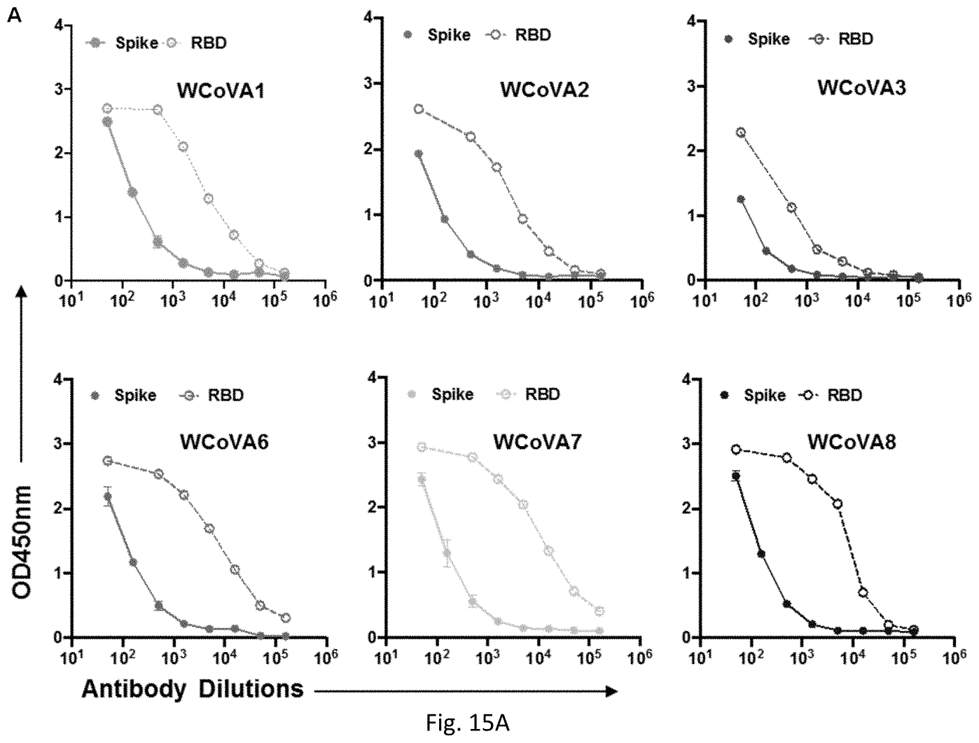

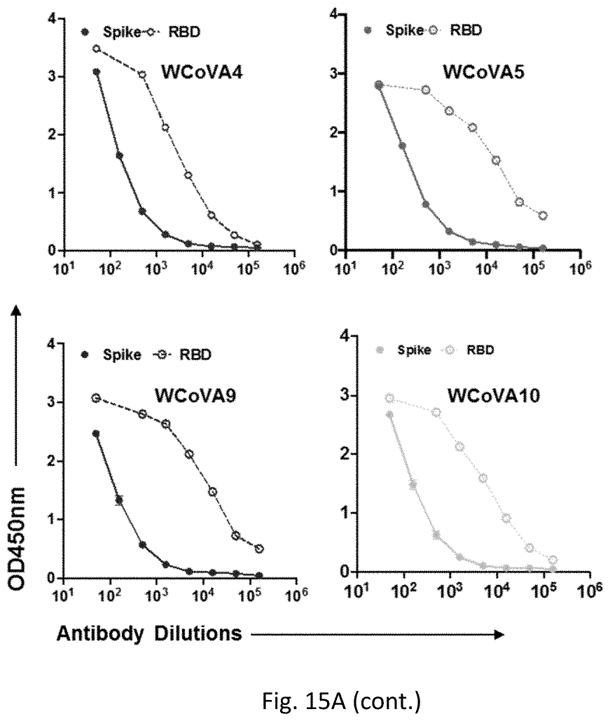

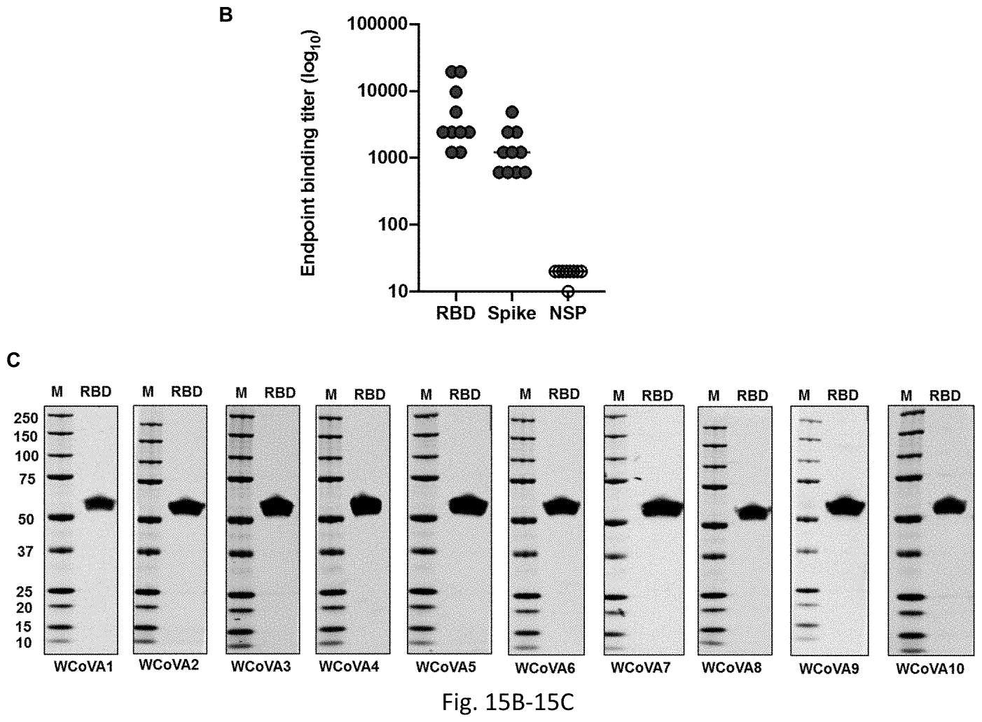

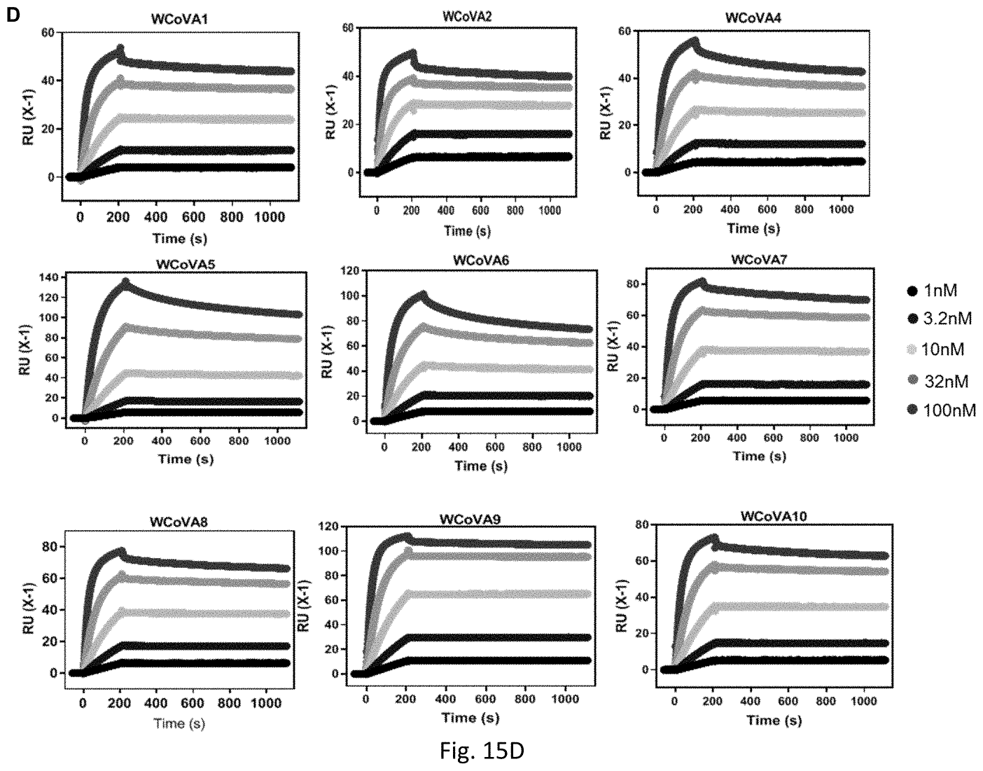

The antibody specificity was confirmed by Western blot analysis for the heavy and light chain expression (FIG. 14E). Further, the ability of these mAbs to bind to SARS-CoV-2 full length Spike as well as RBD was investigated by indirect ELISA. The results showed that all the mAbs specifically and strongly bound to both full-length Spike and RBD proteins, whereas no binding was observed to the non-specific (NSP) control. FIG. 15A shows a dose-dependent binding curve for IgG clones represented by the average ELISA signals plotted versus different dilutions of mAb clones. All the clones showed a high end-point titer without any background (FIG. 15B). Furthermore, the binding specificity of mAb clones were also confirmed by Western blot analysis. For this analysis, SARS-CoV-2-RBD protein was loaded and subsequently probed with equal concentrations of mAb clones as mentioned in the materials and methods section. The findings revealed that all the 10 mAb clones bound specifically to SARS-CoV-2-RBD protein (FIG. 15C).

Kinetic Analysis of SARS-CoV-2-Spike Antibodies and their Target by Surface Plasmon Resonance Analysis

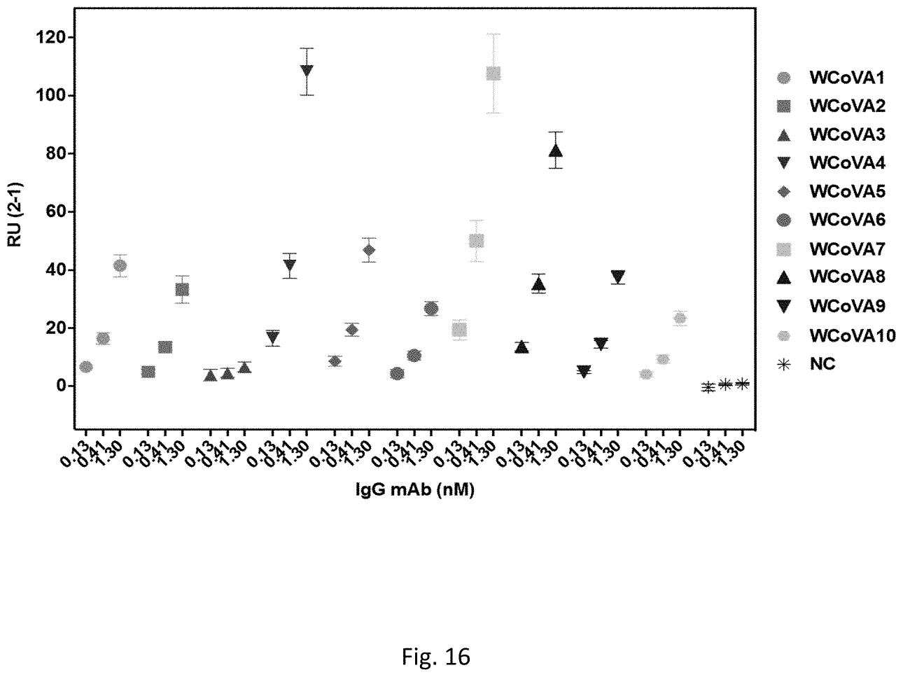

Surface plasmon resonance binding analysis presents an important tool for mAb-antigen binding characterization. The binding kinetics of the mAb clones and their target, RBD regions of the SARS-CoV-2 Spike were analyzed by SPR. Initially, CoV-S-RBD was immobilized on a carboxymethyl dextran sensor chip via amine coupling. Recombinant ACE2 was used to test that the SARS-CoV-2-RBD was functional after immobilization. All the mAb clones were tested at 0.13 nM, 0.41 nM and 1.3 nM concentrations and 9 out of 10 clones showed dose-dependent binding to SARS-CoV-2-RBD (FIG. 16). These 9 mAb clones were then further characterized to determine the kinetic parameters for the interaction. For this experiment, the mAb clones were immobilized on a Protein A/G sensor chip which allows for an estimation of the active antibody immobilized on the chip surface. Target immobilization level for the antibodies was 400 RU, given the MW ratio between the antibody and SARS-CoV-2-RBD, this would result in a theoretical Rmax of 80 RU. The sensograms for these clones are shown in FIG. 15D and the binding kinetic values for all the 9 IgG mAb clones are summarized in Table 1. Very low KD values (<1 nM) were obtained in case of all the 9 IgG mAb clones which strongly indicates their high affinity interaction with SARS-CoV-2-RBD protein. WCoVA9 had both the highest affinity and the slowest dissociation rate. Three of the clones (WCoVA5, WCoVA8 and WCoVA9) had Rmax values greater than 100% of the expected Rmax. This suggests that these antibodies may bind to 2 molecules of SARS-CoV-2-RBD per antibody molecule. Three of the clones (WCoVA1, WCoVA2 and WCoVA4) had Rmax values around 50% of the expected Rmax.

TABLE 1
Binding kinetics of IgG mAb clones and their target SARS-CoV-2-RBD
analyzed by SPR through immobilization of SARS-CoV-2-RBD using
standard amine coupling.

| | | | | | | | | % |
|---|---|---|---|---|---|---|---|---|
| | | | | | | Mean | | Active |
| | Kₒₙ | k_{off} | t_{1/2} | K_{D} | Rₘₐₓ | Capture | Expected | IgG |
| Clone | (×10⁵M⁻¹s⁻¹) | (×10⁻⁴s⁻1) | (min) | (nM) | (RU) | Level | Rmax | (1:1) |
| WCoVA1 | 3.7 | 0.78 | 148 | 0.21 | 45 | 405 | 81 | 56 |
| WCoVA2 | 7 | 0.72 | 160 | 0.10 | 40 | 445 | 89 | 45 |
| WCoVA4 | 4.1 | 1.6 | 72 | 0.38 | 47 | 438 | 88 | 54 |
| WCoVA5 | 2.1 | 2 | 58 | 0.95 | 125 | 408 | 82 | 153 |
| WCoVA6 | 3.7 | 2.2 | 53 | 0.59 | 86 | 458 | 92 | 94 |
| WCoVA7 | 3.3 | 0.93 | 124 | 0.28 | 74 | 380 | 76 | 97 |
| WCoVA8 | 3.9 | 0.82 | 140 | 0.21 | 69 | 250 | 50 | 138 |
| WCoVA9 | 4.7 | 0.19 | 607 | 0.042 | 104 | 390 | 78 | 133 |
| WCoVA10 | 3.6 | 0.70 | 165 | 0.19 | 66 | 380 | 76 | 87 |

Angiotensin-Converting Enzyme 2 (ACE2) Inhibition by SARS-CoV-2-Spike mAbs

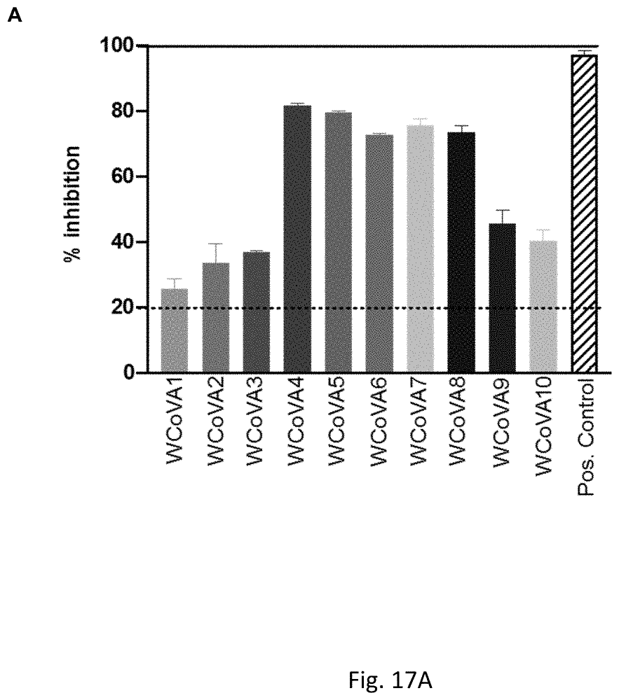

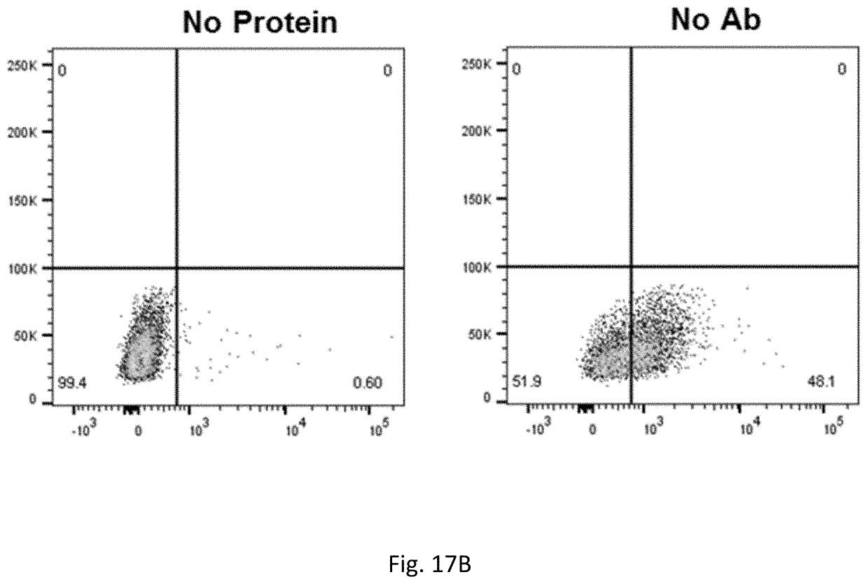

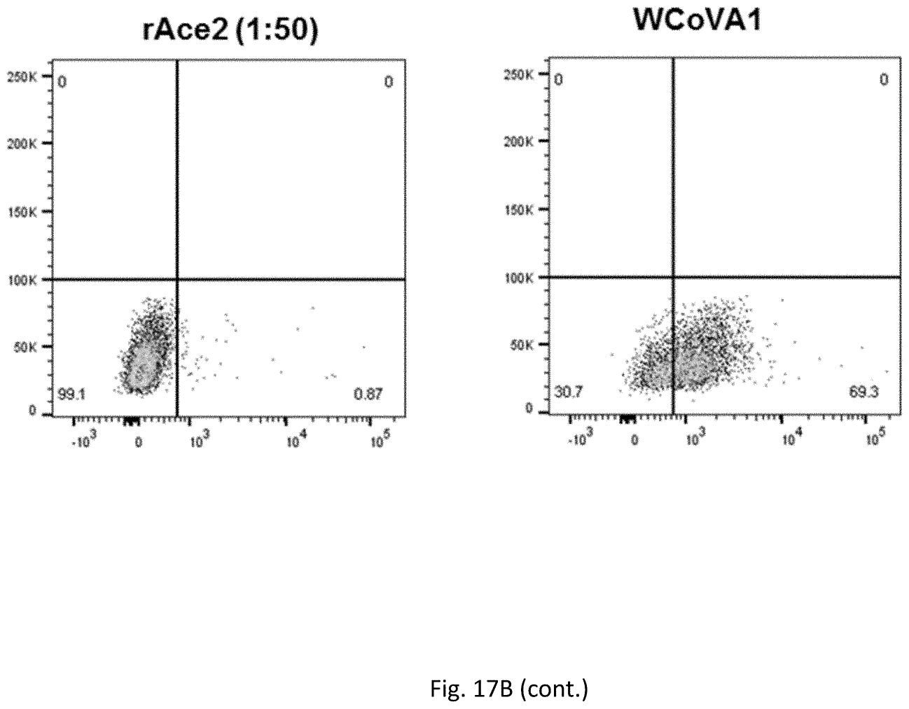

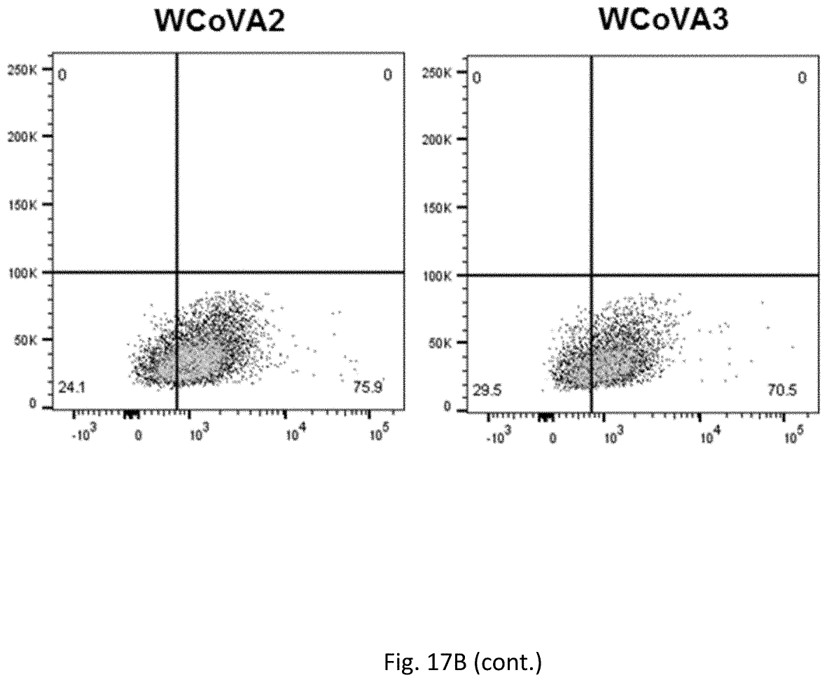

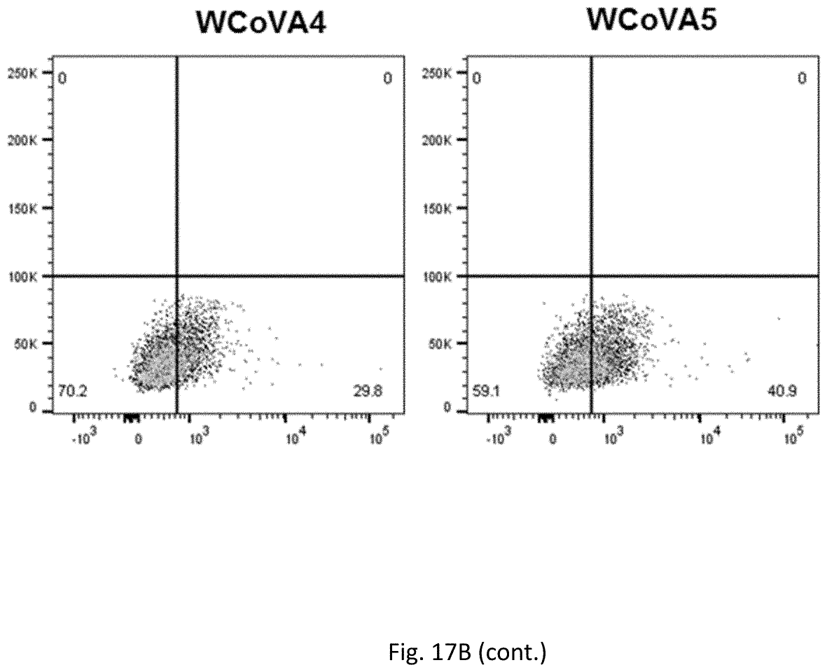

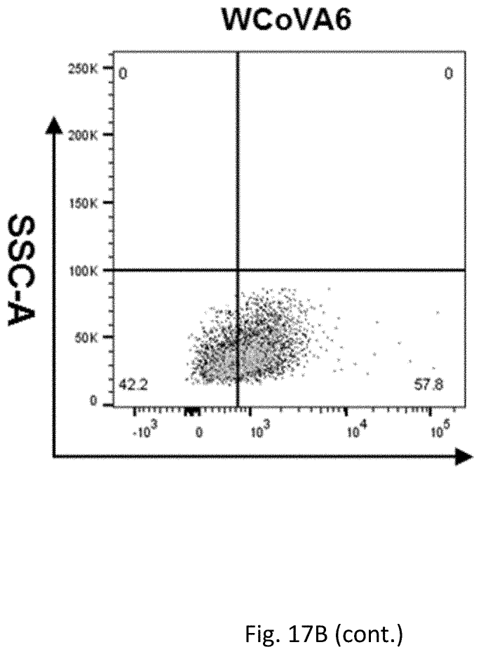

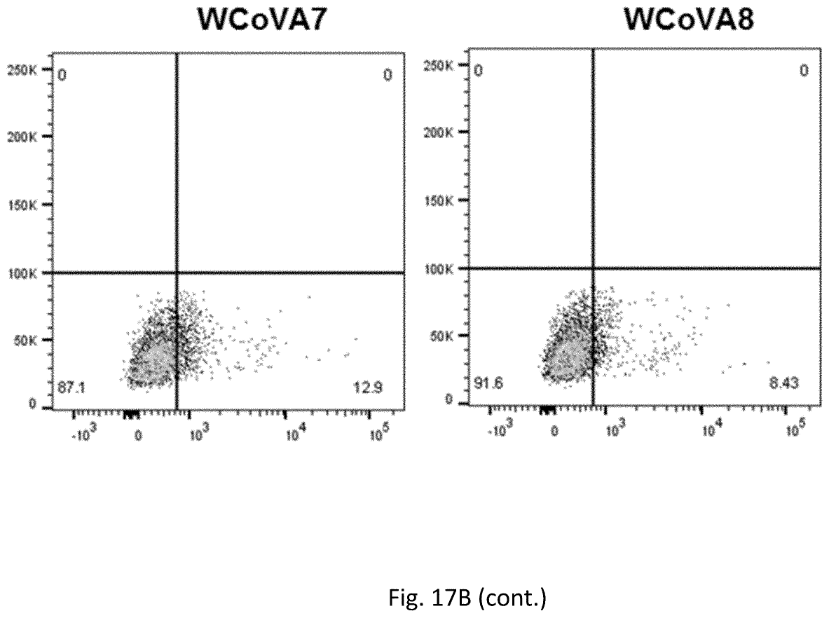

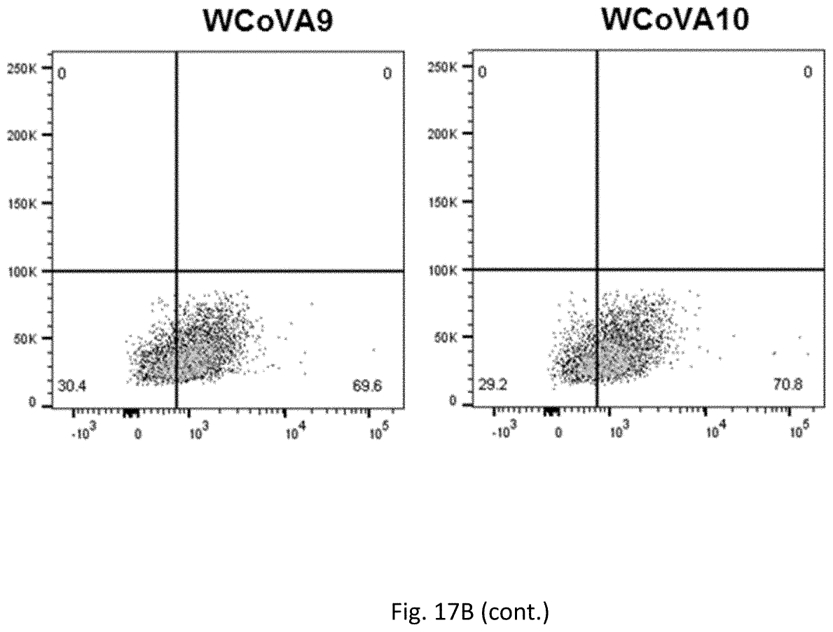

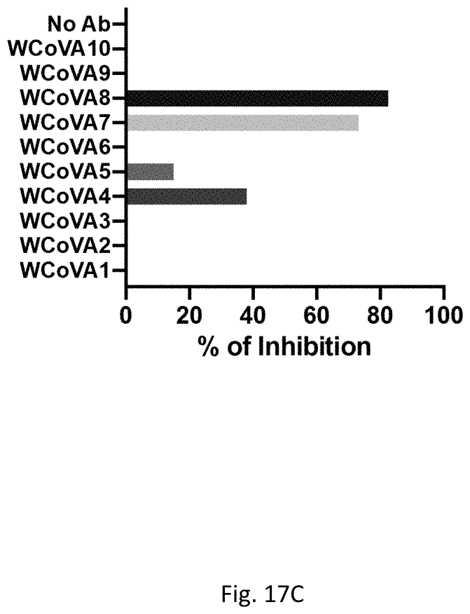

Shang and group reported on the crystal structure of the SARS-CoV-2 spike protein's RBD in complex with ACE2 in their study and showed that the ACE2-binding ridge in SARS-CoV-2 RBD possess a compact conformation (Shang et al., 2020). NAbs with the ability to target the SARS-CoV-2 RBD have the potential to block ACE2 binding by the virus, and prevent viral entry and possibly protect cells therapeutically from infection (Walker et al., 2020). Therefore, therapeutically active mAbs targeting the interaction between the SARS-CoV-2 Spike protein and the ACE2 receptor is of interest for screening the hybridomas. Hence, whether these mAbs can block the interaction between SARS-CoV-2-RBD and ACE2 was evaluated with the help of a blocking ELISA. The results showed that that the mAb clones can efficiently block SARS-CoV-2-RBD-ACE2 interaction with variable efficiency (FIG. 17A). A total of 5 clones (WCoVA4, WCoVA5, WCoVA6, WCoVA7 and WCoVA8) were able to cause more than 70% inhibition of SARS-CoV-2-RBD and ACE2 interaction in this assay. The potential of the identified SARS-CoV-2-Spike IgG mAbs as inhibitors of viral entry was then evaluated. For this purpose, a flow cytometry-based assay was used to test the interference of these mAb clones with the binding of the SARS-CoV-2 Spike protein to CHO-ACE2 cells. Four mAb clones (WCoVA4, WCoVA5, WCoVA7 and WCoVA8) blocked the binding of SARS-Spike to ACE2 as shown by flow cytometry (FIG. 17B and FIG. 17C).

Neutralization of SARS-CoV-2 Pseudovirus by SARS-CoV-2-Spike Antibodies

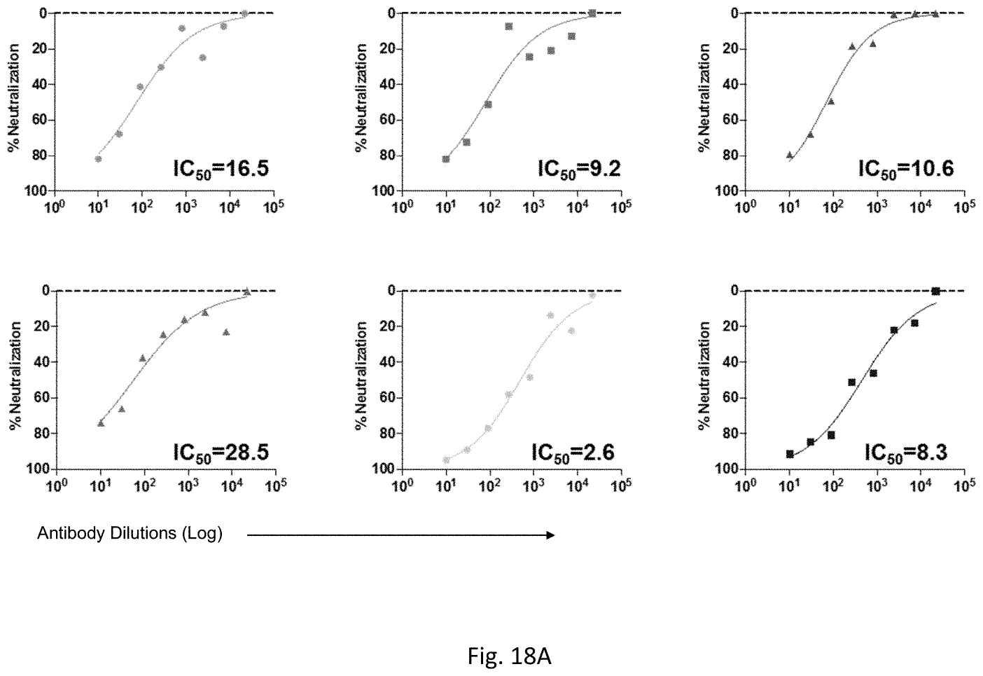

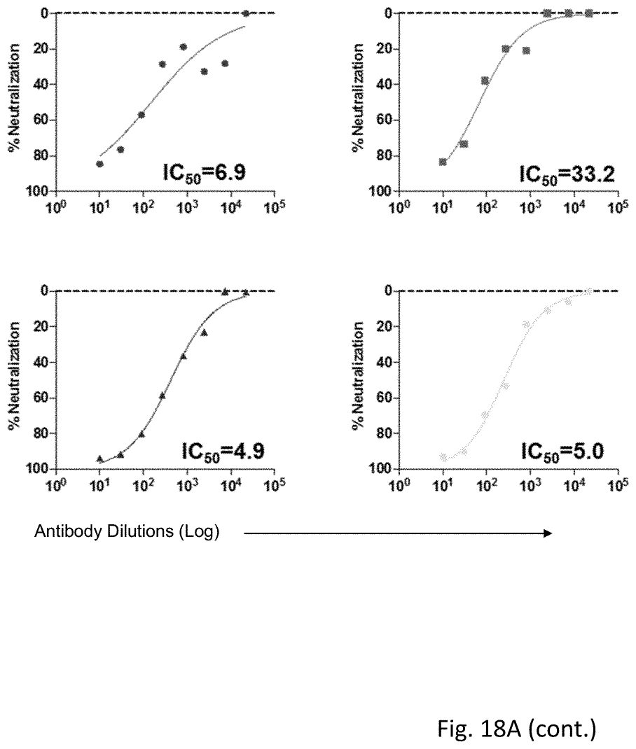

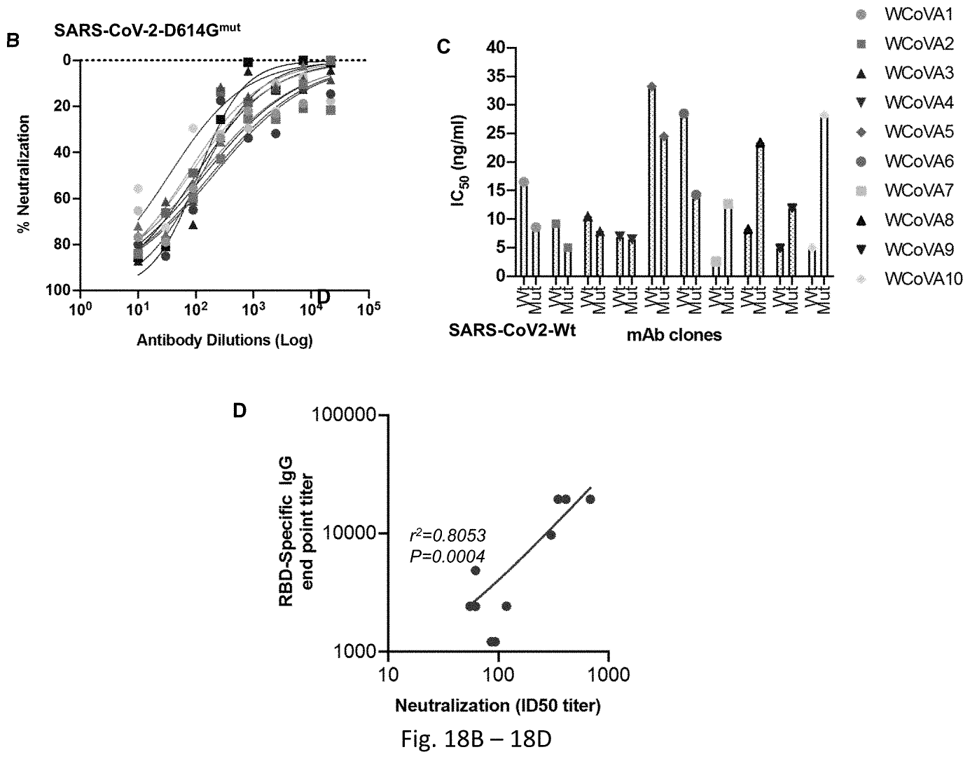

NAbs possess the ability to inhibit viral infection through blockage of the replication cycle of virus (Tong et al., 2020). Therefore, the functionalities of the SARS-CoV-2-Spike mAbs were evaluated using a pseudovirus neutralization assay developed (Smith et al., 2020). As expected, pseudovirus neutralization was observed by positive control ACE2-Ig with an IC50 value of 0.9 μg/mL, but not in the case of negative control murine antibody TA99. Antibodies secreted by 10 out of 10 IgG mAb clones were capable of neutralizing more than 50% of both the wild type (FIG. 18A) and D614G mutated virus (FIG. 18B) at the lowest sample dilution (10-fold). In addition, the IC50 values of these clones were assessed, which suggested them to effectively block infection. mAb clones; WCoVA4, WCoVA7, WCoVA9 and WCoVA10 were observed to be the most potent against the wild type virus, with IC50 values of 6.9, 2.6, 4.9 and 5.0 ng/ml respectively. Further, WCoVA1, WCoVA2, WCoVA3 and WCoVA4 were found to be of higher efficacy against D614G mutated virus with IC50 values of 8.85, 5.05, 7.93 and 6.42 ng/ml respectively (FIG. 18C). Also, a significant correlation between ELISA titers and neutralization titers of these SARS-CoV-2-Spike mAbs was observed (FIG. 18D). These results suggested increased antiviral activity of these mAbs to be related to their increased binding affinity with RBD, but their detailed interactions require further studies.

Glycomic Analysis of SARS-CoV-2 Antibodies

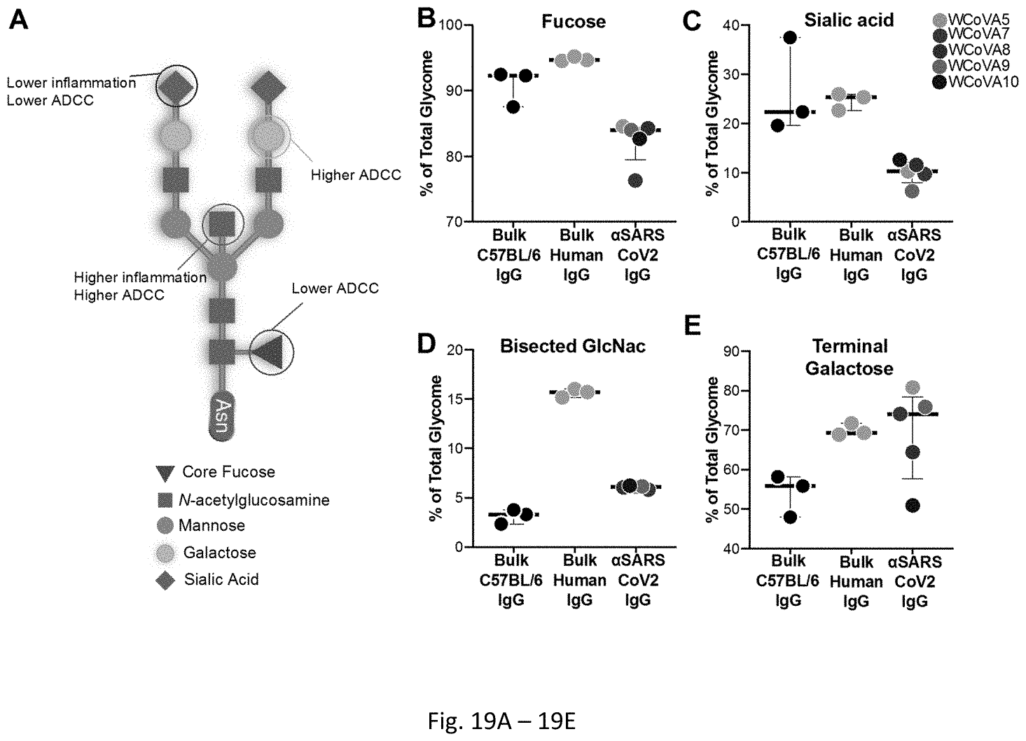

Non-neutralizing Fc-mediated effector functions of antibodies, including antibody-dependent cellular cytotoxicity (ADCC), play an important role in controlling viral infections (Alpert et al., 2012; Baum et al., 1996; Bhiman and Lynch, 2017; Bruel et al., 2016; Chung et al., 2011; Halper-Stromberg and Nussenzweig, 2016; Isitman et al., 2016; Lee and Kent, 2018; Madhavi et al., 2015). Antibody glycosylation strongly impacts its effector functions. The presence of core fucose results in a weaker binding to Fcγ receptor IIIA and reduces ADCC (Masuda et al., 2007). Although core fucose has the most significant impact on ADCC, other glycomic features have also been shown to impact ADCC: terminal sialic acid reduces ADCC, (Naso et al., 2010; Raju, 2008; Raju and Scallon, 2007) bisecting GlcNAc induces both innate immune function and inflammation (Davies et al., 2001; Takahashi et al., 2009; Umana et al., 1999) and terminal galactose induces ADCC (FIG. 19A) (Thomann et al., 2016). The glycosylation of WCoVA5, WCoVA7, WCoVA8, WCoVA9, and WCoVA10 was examined. All of these antibodies (especially WCoVA7) have lower levels of core fucose, lower levels of sialic acid, and higher levels of bisecting GlcNac compared to bulk C57BL/6 IgG (FIG. 19B-D). In addition, WCoVA5, WCoVA7, and WCoVA9 have high levels of terminal galactose (FIG. 19B-E). This glycomic profile (especially for WCoVA7) suggests that these antibodies would have high Fc-mediated effector functions.

Table 2 provides an overview of the characteristics of the down selected antibodies.

TABLE 2
Characteristics of the down selected antibodies

| | | | | | | ACE2 | Neutral- | Neutral- |
|---|---|---|---|---|---|---|---|---|
| | | | | | ACE2 | blocking | ization of | ization of |
| | Spike | RBD | Specificity | | blocking | by Flow | SARS- | SARS- |
| Antibody | Binding | binding | by | SPR by | by | based | CoV2- | CoV2- |
| Clones | ELISA | ELISA | WB | Biacore | ELISA | assay | WT | D614G^{mut} |
| WCoVA1 | +++ | ++ | +++ | ++ | + | − | + | ++ |
| WCoVA2 | ++ | ++ | +++ | +++ | + | − | ++ | +++ |
| WCoVA3 | + | + | +++ | − | + | − | ++ | ++ |
| WCoVA4 | +++ | +++ | +++ | ++ | +++ | ++ | +++ | +++ |
| WCoVA5 | +++ | ++ | +++ | + | +++ | + | + | + |
| WCoVA6 | ++ | ++ | +++ | + | +++ | − | + | + |
| WCoVA7 | ++ | +++ | +++ | ++ | +++ | +++ | +++ | ++ |
| WCoVA8 | ++ | +++ | +++ | ++ | +++ | +++ | ++ | + |
| WCoVA9 | ++ | +++ | +++ | +++ | ++ | − | +++ | ++ |
| WCoVA10 | +++ | +++ | +++ | +++ | ++ | − | +++ | + |

Computational Characterization of Antibody-Antigen Docking

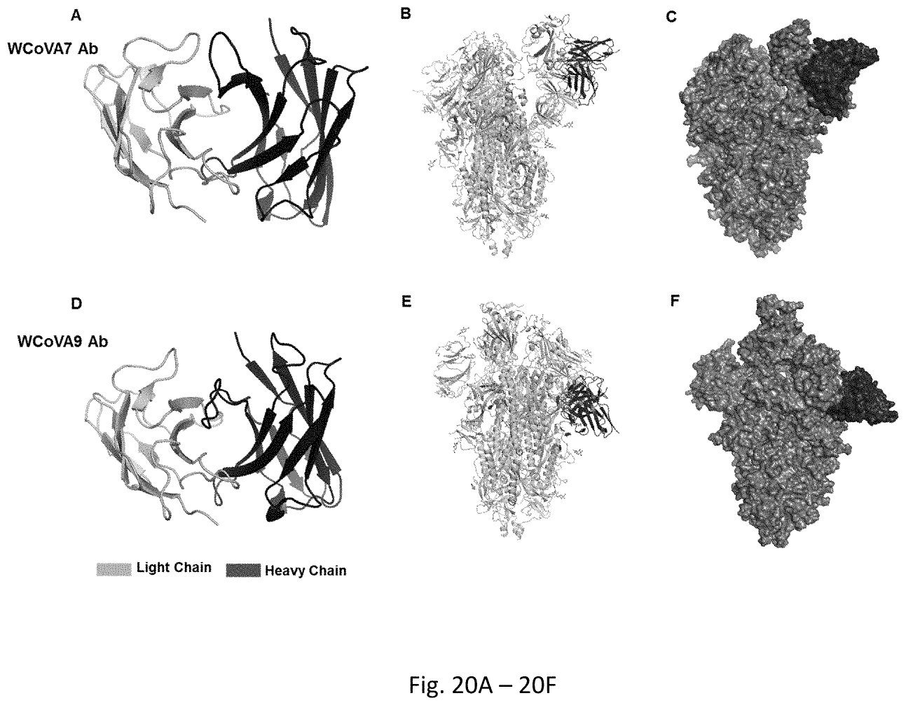

Based on overall specificity, clones WCoVA7 and WCoVA9 were selected for sequencing and both heavy and light chains were amplified. These sequences were then used as tools for antibody-antigen docking. The structures of WCoVA7 and WCoVA9 variable regions were predicted using AbYmod. SARS-CoV-2 spike structure was obtained from the protein data bank (PDB: 6VYB), and modeling of SARS-CoV-2 spike protein with antibody complexes was performed at ZDOCK server. ZDOCK performs an exhaustive, grid-based search for the docking modes of two component proteins (Pierce et al., 2011). SARS-CoV-2 spike protein was stationary in the output predictions while antibodies were moved. Antibody-antigen docking reveled that, WCoVA7 antibody bound to the interface of RBD domain (FIG. 20A-C), whereas WCoVA9 antibody bound to the interface between N-terminal domain (NTD) and RBD domain (FIG. 20B).

TABLE 3
IMGT Analysis of V(D)J Junctions for WCoVA7

| | | | V- | | | | |
|---|---|---|---|---|---|---|---|
| | V-GENE | | REGION | J-GENE | D-GENE | | |
| | and | | identity | and | and | AA | Junction |
| Sequence | allele | Functionality | % (nt) | allele | allele | Junction | frame |
| V_{H} | Musmus | productive | 100.00% | Musmus | Musmus | CARIYY | in-frame |
| | IGHV4- | | (288/288 | IGHJ4*01 F | IGHD2- | GSPGA | |
| | 1*02 F | | nt) | | 2*01 F | MDYW | |
| V_{L} | Musmus | productive | 99.28% | Musmus | — | CQQDY | in-frame |
| | IGKV6- | | (277/279 | IGKJ2*01 F | | FSPYT | |
| | 32*01 F | | nt) | | | F | |

TABLE 4
IMGT Analysis of V(D)J Junctions for WCoVA9

| | | | V- | | | | |
|---|---|---|---|---|---|---|---|
| | V-GENE | | REGION | J-GENE | D-GENE | | |
| | and | Function- | identity % | and | and | AA | Junction |
| Sequence | allele | ality | (nt) | allele | allele | Junction | frame |
| V_{H} | Musmus | Productive | 98.96% | Musmus | Musmus | CAPYY | in-frame |
| | IGHV14- | | (285/288 | IGHJ4*01 F | IGHD1- | DYVGA | |
| | 3*02 F | | nt) | | 2*01 F | MDYW | |
| V_{L} | Musmus | productive | 99.64% | Musmus | — | CQQLY | in-frame |
| | IGKV12- | | (278/279 | IGKJ1*01 F | | NTPLTF | |
| | 98*01 F | | nt) | | | | |

### Example 4: Antibody and DMAb Sequences

| SEQ | | |
|---|---|---|
| ID | | |
| NO: | Sequence Type | Description |
| 1 | DNA | Optimized CR3022 SARS-CoV-2 DMAb |
| | | heavy chain |
| 2 | Amino Acid | Optimized CR3022 SARS-CoV-2 DMAb |
| | | heavy chain |
| 3 | DNA | Optimized CR3022 SARS-CoV-2 DMAb light |
| | | chain |
| 4 | Amino Acid | Optimized CR3022 SARS-CoV-2 DMAb light |
| | | chain |
| 5 | DNA | Clone 1F8 Heavy Chain |
| 6 | Amino Acid | Clone 1F8 Heavy Chain |
| 7 | DNA | Clone 1F8 Light Chain |
| 8 | Amino Acid | Clone 1F8 Light Chain |
| 9 | DNA | 1F8 full length DMAb |
| 10 | Amino Acid | 1F8 full length DMAb |
| 11 | DNA | Clone 3B12 Heavy Chain |
| 12 | Amino Acid | Clone 3B12 Heavy Chain |
| 13 | DNA | Clone 3B12 Light Chain |
| 14 | Amino Acid | Clone 3B12 Light Chain |
| 15 | DNA | 3B12 full length DMAb |
| 16 | Amino Acid | 3B12 full length DMAb |
| 17 | DNA | Clone 3B11 Heavy Chain |
| 18 | Amino Acid | Clone 3B11 Heavy Chain |
| 19 | DNA | Clone 3B11 Light Chain |
| 20 | Amino Acid | Clone 3B11 Light Chain |
| 21 | DNA | 3B11 full length DMAb |
| 22 | Amino Acid | 3B11 full length DMAb |
| 23 | DNA | 256 (80R) Heavy Chain CDR1 |
| 24 | DNA | 256 (80R) Heavy Chain CDR2 |
| 25 | DNA | 256 (80R) Heavy Chain CDR3 |
| 26 | Amino Acid | 256 (80R) Heavy Chain CDR1 |
| 27 | Amino Acid | 256 (80R) Heavy Chain CDR2 |
| 28 | Amino Acid | 256 (80R) Heavy Chain CDR3 |
| 29 | DNA | 256 (80R) Heavy Chain |
| 30 | Amino Acid | 256 (80R) Heavy Chain |
| 31 | DNA | 256 (80R) Light Chain CDR1 |
| 32 | DNA | 256 (80R) Light Chain CDR2 |
| 33 | DNA | 256 (80R) Light Chain CDR3 |
| 34 | Amino Acid | 256 (80R) Light Chain CDR1 |
| 35 | Amino Acid | 256 (80R) Light Chain CDR2 |
| 36 | Amino Acid | 256 (80R) Light Chain CDR3 |
| 37 | DNA | 256 (80R) Light Chain |
| 38 | Amino Acid | 256 (80R) Light Chain |
| 39 | DNA | 256 (80R) full length DMAb |
| 40 | Amino Acid | 256 (80R) full length DMAb |
| 41 | DNA | S309 SARS-CoV-2 DMAb heavy chain |
| 42 | Amino Acid | S309 SARS-CoV-2 DMAb heavy chain |
| 43 | DNA | S309 SARS-CoV-2 DMAb light chain |
| 44 | Amino Acid | S309 SARS-CoV-2 DMAb light chain |
| 45 | DNA | 2130_WT/WT_1plasmid |
| 46 | Amino Acid | 2130_WT/WT_1plasmid |
| 47 | DNA | 2130_WT/G_1plasmid |
| 48 | Amino Acid | 2130_WT/G_1plasmid |
| 49 | DNA | 2130_WT/GK_1plasmid |
| 50 | Amino Acid | 2130_WT/GK_1plasmid |
| 51 | DNA | 2130_WT/TM_1plasmid |
| 52 | Amino Acid | 2130_WT/TM_1plasmid |
| 53 | DNA | 2381_WT/WT_1plasmid |
| 54 | Amino Acid | 2381_WT/WT_1plasmid |
| 55 | DNA | 2381_WT/G_1plasmid |
| 56 | Amino Acid | 2381_WT/G_1plasmid |
| 57 | DNA | 2381_WT/GK_1plasmid |
| 58 | Amino Acid | 2381_WT/GK_1plasmid |
| 59 | DNA | 2381_WT/TM_1plasmid |
| 60 | Amino Acid | 2381_WT/TM_1plasmid |
| 61 | DNA | 2196_WT/WT_1plasmid |
| 62 | Amino Acid | 2196_WT/WT_1plasmid |
| 63 | DNA | 2196_WT/G_1plasmid |
| 64 | Amino Acid | 2196_WT/G_1plasmid |
| 65 | DNA | 2196_WT/GK_1plasmid |
| 66 | Amino Acid | 2196_WT/GK_1plasmid |
| 67 | DNA | 2196_WT/TM_1plasmid |
| 68 | Amino Acid | 2196_WT/TM_1plasmid |
| 69 | DNA | 2196_MOD/WT_1plasmid |
| 70 | Amino Acid | 2196_MOD/WT_1plasmid |
| 71 | DNA | 2196_MOD/G_1plasmid |
| 72 | Amino Acid | 2196_MOD/G_1plasmid |
| 73 | DNA | 2196_MOD/GK_1plasmid |
| 74 | Amino Acid | 2196_MOD/GK_1plasmid |
| 75 | DNA | 2196_MOD/TM_1plasmid |
| 76 | Amino Acid | 2196_MOD/TM_1plasmid |
| 77 | DNA | WCoVA7 Heavy Chain CDR1 |
| 78 | DNA | WCoVA7 Heavy Chain CDR2 |
| 79 | DNA | WCoVA7 Heavy Chain CDR3 |
| 80 | Amino Acid | WCoVA7 Heavy Chain CDR1 |
| 81 | Amino Acid | WCoVA7 Heavy Chain CDR2 |
| 82 | Amino Acid | WCoVA7 Heavy Chain CDR3 |
| 83 | DNA | WCoVA7 Heavy Chain |
| 84 | Amino Acid | WCoVA7 Heavy Chain |
| 85 | DNA | WCoVA7 Light Chain CDR1 |
| 86 | DNA | WCoVA7 Light Chain CDR2 |
| 87 | DNA | WCoVA7 Light Chain CDR3 |
| 88 | Amino Acid | WCoVA7 Light Chain CDR1 |
| 89 | Amino Acid | WCoVA7 Light Chain CDR2 |
| 90 | Amino Acid | WCoVA7 Light Chain CDR3 |
| 91 | DNA | WCoVA7 Light Chain |
| 92 | Amino Acid | WCoVA7 Light Chain |
| 93 | DNA | WCoVA7 dMAB Heavy Chain (mIgG1Kappa) |
| 94 | Amino Acid | WCoVA7 dMAB Heavy Chain (mIgG1Kappa) |
| 95 | DNA | WCoVA7 dMAB Light Chain (mIgG1Kappa) |
| 96 | Amino Acid | WCoVA7 dMAB Light Chain (mIgG1Kappa) |
| 97 | DNA | WCoVA7 full length dMAB (mIgG1Kappa) |
| 98 | Amino Acid | WCoVA7 full length dMAB (mIgG1Kappa) |
| 99 | DNA | WCoVA7 dMAB Heavy Chain (hIgG1Kappa) |
| 100 | Amino Acid | WCoVA7 dMAB Heavy Chain (hIgG1Kappa) |
| 101 | DNA | WCoVA7 dMAB Light Chain (hIgG1Kappa) |
| 102 | Amino Acid | WCoVA7 dMAB Light Chain (hIgG1Kappa) |
| 103 | DNA | WCoVA7 full length dMAB (hIgG1Kappa) |
| 104 | Amino Acid | WCoVA7 full length dMAB (hIgG1Kappa) |
| 105 | DNA | WCoVA9 Heavy Chain CDR1 |
| 106 | DNA | WCoVA9 Heavy Chain CDR2 |
| 107 | DNA | WCoVA9 Heavy Chain CDR3 |
| 108 | Amino Acid | WCoVA9 Heavy Chain CDR1 |
| 109 | Amino Acid | WCoVA9 Heavy Chain CDR2 |
| 110 | Amino Acid | WCoVA9 Heavy Chain CDR3 |
| 111 | DNA | WCoVA9 Heavy Chain |
| 112 | Amino Acid | WCoVA9 Heavy Chain |
| 113 | DNA | WCoVA9 Light Chain CDR1 |
| 114 | DNA | WCoVA9 Light Chain CDR2 |
| 115 | DNA | WCoVA9 Light Chain CDR3 |
| 116 | Amino Acid | WCoVA9 Light Chain CDR1 |
| 117 | Amino Acid | WCoVA9 Light Chain CDR2 |
| 118 | Amino Acid | WCoVA9 Light Chain CDR3 |
| 119 | DNA | WCoVA9 Light Chain |
| 120 | Amino Acid | WCoVA9 Light Chain |
| 121 | DNA | WCoVA9 dMAB Heavy Chain |
| | | (mIgG2bKappa) |
| 122 | Amino Acid | WCoVA9 dMAB Heavy Chain |
| | | (mIgG2bKappa) |
| 123 | DNA | WCoVA9 dMAB Light Chain (mIgG2bKappa) |
| 124 | Amino Acid | WCoVA9 dMAB Light Chain (mIgG2bKappa) |
| 125 | DNA | WCoVA9 full length dMAB (mIgG2bKappa) |
| 126 | Amino Acid | WCoVA9 full length dMAB (mIgG2bKappa) |
| 127 | DNA | WCoVA9 dMAB Heavy Chain (hIgG2bKappa) |
| 128 | Amino Acid | WCoVA9 dMAB Heavy Chain (hIgG2bKappa) |
| 129 | DNA | WCoVA9 dMAB Light Chain (hIgG2bKappa) |
| 130 | Amino Acid | WCoVA9 dMAB Light Chain (hIgG2bKappa) |
| 131 | DNA | WCoVA9 full length dMAB (hIgG2bKappa) |
| 132 | Amino Acid | WCoVA9 full length dMAB (hIgG2bKappa) |

### Example 5: Rapid Development of a Synthetic DNA Vaccine for COVID-19

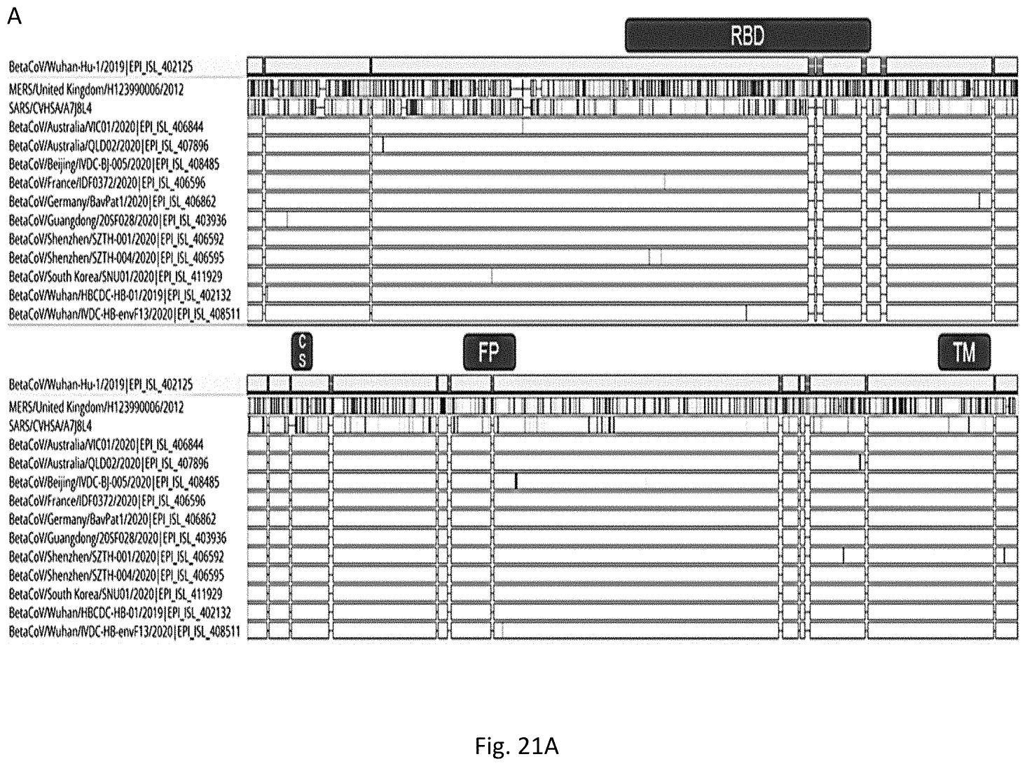

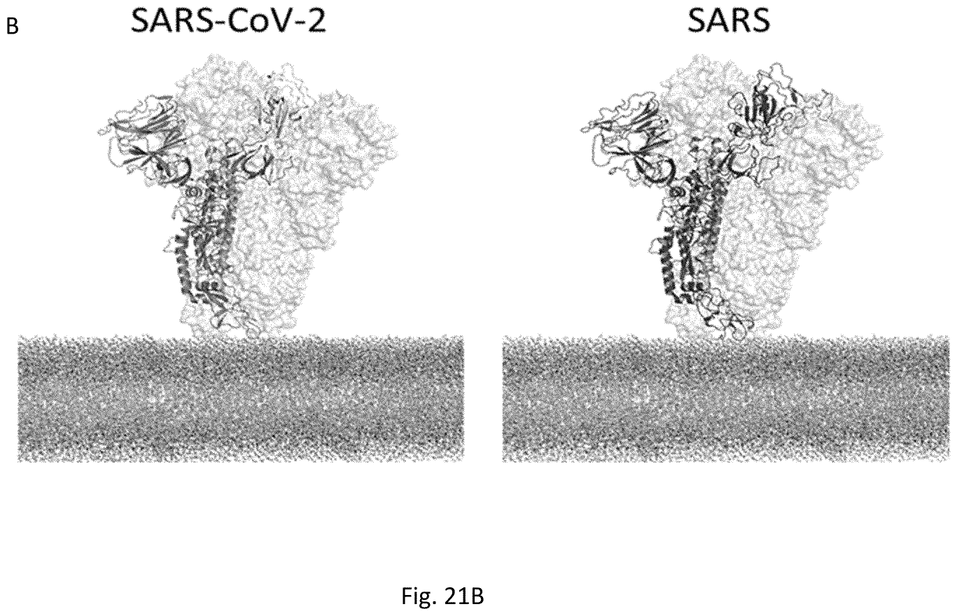

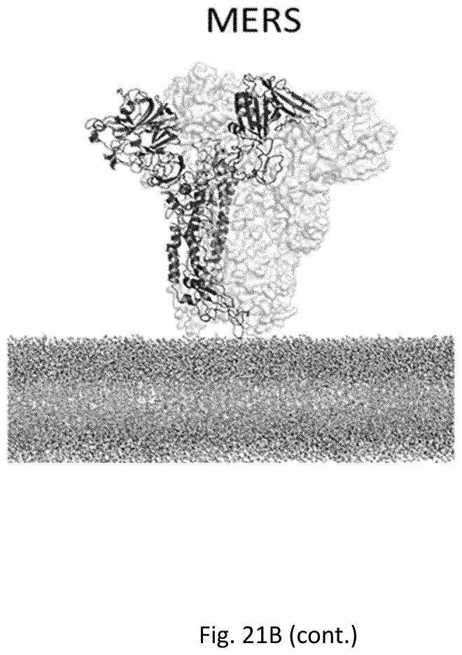

The SARS-CoV-2 spike is most similar in sequence and structure to SARS-CoV spike protein (Wrapp et al., 2020, Science, eabb2507), and shares a global protein fold architecture with the MERS-CoV spike protein (FIG. 21). Unlike glycoproteins of HIV and influenza, the prefusion form of the coronavirus trimeric spike is conformationally dynamic, fully exposing the receptor-binding site infrequently (Kirchdoerfer et al., 2018, Sci Rep, 8:15701). The receptor-binding site is a vulnerable target for neutralizing antibodies. In fact, MERS nAbs targeted at the receptor-binding domain (RBD) tend to have greater neutralizing potency than other epitopes (Wang et al., 2018, J Virol, 92:e02002-17). A recent report demonstrated that one neutralizing anti-SARS antibody could cross-react to the RBD of SARS-CoV-2 (Tian et al., 2020, Emerg Microbes Infect, 9:382-385). These data suggest that the SARS-CoV-2 RBD is an important target for vaccine development. Recent data has revealed SARS-CoV-2 S protein binds the same host receptor, angiotensin-converting enzyme 2 (ACE-2) as SARS-CoV S protein (Wrapp et al., 2020, Science, eabb2507).

Here, the design and initial preclinical testing of SARS-CoV-2 synthetic DNA vaccine candidates are described. Expression of the SARS-CoV-2 S antigen RNA and protein are shown after in vitro transfection of COS and 293T cells, respectively with the vaccine candidates. The induction of immunity by the selected immunogen was followed in mice and guinea pigs, measuring SARS-CoV-2 S protein-specific antibody levels in serum and in the lung fluid, and competitive inhibition of ACE2 binding. The INO-4800 vaccine induces cellular and humoral host immune responses that can be observed within days following a single immunization, including cross-reactive responses against SARS-CoV. Taken together, the data demonstrate the immunogenicity of this SARS-CoV-2 synthetic DNA vaccine candidate, targeting the SARS-CoV-2 S protein, supporting further translational studies to rapidly accelerate the development of this candidate to respond to the current global health crisis.

The novel coronavirus, SARS-CoV-2, and associated COVID-19 disease is rapidly spreading, and has become a global pandemic. Here, accelerated preclinical development of a synthetic DNA-based SARS-CoV-2 vaccine, INO-4800, is described to combat this emerging infectious disease. Synthetic DNA vaccine design and synthesis was immediately initiated upon public release of the SARS-CoV-2 genome sequences on Jan. 11, 2020. The majority of the in vitro and in vivo studies described herein were executed within 6 weeks of the SARS-CoV-2 genome sequence becoming available. The data support the expression and immunogenicity of the INO-4800 synthetic DNA vaccine candidate in multiple animal models. Humoral and T cells responses were observed in mice after a single dose. In guinea pigs clinical delivery parameters were employed and antibody titers were observed after a single dose.

Halting a rapidly emerging infectious disease requires an orchestrated response from the global health community and requires improved strategies to accelerate vaccine development. In response to the 2019/2020 coronavirus outbreak the highly adaptable synthetic DNA medicine platform was rapidly employed. The design and manufacture of synthetic DNA vaccines for novel antigens is a process in which the target antigen sequence is inserted into a highly characterized and clinically-tested plasmid vector backbone (pGX0001). The construct design and engineering parameters are optimized for in vivo gene expression.

SARS-CoV-2 S protein was chosen as the antigen target. The SARS-CoV-2 S protein is a class I membrane fusion protein, which the major envelope protein on the surface of coronaviruses. Initial studies indicate that SARS-CoV-2 interaction with its host receptor (ACE-2) can be blocked by antibodies (Zhou et al., 2020, Nature, 579:270-273). In vivo immunogenicity studies in both mouse and guinea pig models revealed levels of S protein-reactive IgG in the serum of INO-4800 immunized animals. In addition to full-length S1+S2 and S1, INO-4800 immunization induced RBD binding antibodies, a domain known to be a target for neutralizing antibodies from SARS-CoV convalescent patients (Zhu et al., 2007, Proc Natl Acad Sci USA, 104:12123-12128; He et al., 2005, Virology, 334:74-82). The experiments presented herein further demonstrate the functionality of these antibodies through competitive inhibition of SARS-CoV-2 spike protein binding to the ACE2 receptor in the presence of sera from INO-4800 immunized animals. Importantly, anti-SARS-CoV-2 binding antibodies were detected in lung washes of INO-4800-immunized mice and guinea pigs. The presence of these antibodies in the lungs has the potential to protect against infection of these tissues and prevent LRD, which is associated with the severe cases of COVID-19. In addition to humoral responses, cellular immune responses have been shown to be associated with more favorable recovery in MERS-CoV infection (Zhao et al., Sci Immunol, 2:eaan5393), and are likely to be important against SARS-CoV infection (Oh et al., 2012, Emerg Microbes Infect, 1:e23). Here, the experiments showed the induction of T cell responses against SARS-CoV-2 as early as day 7 post-vaccine delivery. Rapid cellular responses have the potential to lower viral load and could potentially reduce the spread of SARS-CoV-2 and the associated COVID-19 illness.

In addition to the ability of INO-4800 to rapidly elicit humoral and cellular responses following a single immunization, the synthetic DNA medicine platform has several synergistic characteristics which position it well to respond to disease outbreaks, such as COVID-19. As mentioned previously, the ability to design and synthesize candidate vaccine constructs means that in vitro and in vivo testing can potentially begin within days of receiving the viral sequence, allowing for an accelerated response to vaccine development. The well-defined and established production processes for DNA plasmid manufacture result in a rapid and scalable manufacture process which has the potential to circumvent the complexities of conventional vaccine production in eggs or cell culture. The cost of goods related to DNA manufacture is also significantly lower than currently seen for mRNA-based technologies. A report on the stability profile afforded to through the use of the optimized DNA formulation has recently been published (Tebas et al., 2019, J Infect Dis, 220:400-410). The stability characteristics mean that the DNA drug product is non-frozen and can be stored for 4.5+ years at 2-8° C., room temperature for 1 year and 1 week at 37° C., while maintaining potency at temperatures upwards of 60° C. In the context of a pandemic outbreak, the stability profile of a vaccine plays directly to its ability to be deployed and stockpiled in an efficient and executable manner.

Although vaccine-induced immunopathology has been raised as a potential concern for SARS and MERS vaccine candidates, and possibly for SARS-CoV-2 vaccines, these concerns are likely vaccine-platform dependent and, to-date, no evidence of immune pathogenesis has been reported for MERS DNA vaccines in mice or non-human primate models (Muthumani et al., 2015, Sci Transl Med, 7:301ra13) or SARS DNA vaccine in mice (Yang et al., 2004, Nature, 428:561-564). Lung immunopathology characterized by Th2-related eosinophilia has been reported for whole inactivated virus (IV), recombinant protein, peptide, and/or recombinant viral vector vaccines following SARS-CoV challenge (Tseng et al., 2012, PLoS One, 7:e35421; Iwata-Yoshikawa et al., 2014, J Virol, 88:8597-8614; Bolles et al., 2011, J Virol, 85:12201-12215; Yasui et al., 2008, J Immunol, 181:6337-6348; Wang et al., 2016, ACS Infect Dis, 2:361-376), and more recently in a MERS-CoV challenge model (Agrawal et al., 2016, Hum Vaccin Immunother, 12:2351-2356). However, protective efficacy without lung immunopathology has also been reported for SARS-CoV and MERS-CoV vaccines (Yang et al., 2004, Nature, 428:561-564; Muthumani et al., 2015, Sci Transl Med, 7:301ra132; Luo et al., 2018, Virol Sin 33, 201-204; Qin et al., 2006, Vaccine, 24:1028-1034; Roberts et al., 2010, Viral Immunol 23, 509-519; Deng et al., 2018, Emerg Microbes Infect, 7:60; Zhang et al., 2016, Cell Mol Immunol, 13:180-190; Luke et al., 2016, Sci Transl Med, 8:326ra321; Darnell et al., 2007, J Infect Dis, 196:1329-1338). It is important to note the majority of studies demonstrating CoV vaccine-induced immunopathology utilized the BALB/c mouse, a model known to preferentially develop Th2-type responses. The DNA vaccine platform induces Th1-type immune responses and has demonstrated efficacy without immunopathology in models of respiratory infection, including SARS-CoV (Yang et al., 2004, Nature, 428:561-564), MERS-CoV (Muthumani et al., 2015, Sci Transl Med, 7:301ra132), and RSV (Smith et al., 2017, Vaccine, 35:2840-2847). Future studies will assess vaccine-enhanced disease in INO-4800 immunized animals.

Additional preclinical studies are ongoing to further characterize INO-4800 in small and larger animals. Availability of reagents is a major challenge for development of vaccines against newly emerging infectious diseases, and this limited the ability, in this early stage study, to report on the live virus neutralizing activity of the antibodies in animal models. However, it is reported herein that INO-4800 induced antibodies block SARS-CoV-2 Spike binding to the host receptor ACE2, using a surrogate neutralization assay. Additional studies with live virus neutralizing assays are informative for the investigation of antibody functionality, and demonstrate the ability of INO-4800 immunization to mediate protection of animals against viral challenge.

In summary, the result describing the immunogenicity of SARS-CoV-2 vaccine candidate, INO-4800 are promising, and it is particularly encouraging to measure antibody and T cell levels at an early time point after a single dose of the vaccine supporting the further evaluation of this vaccine.

The material and methods used for the experiments are now described

Cell Lines

Human embryonic kidney (HEK)-293T and COS-7 cell lines were obtained from ATCC (Old Town Manassas, Va.). All cell lines were maintained in DMEM supplemented with 10% fetal bovine serum (FBS) and penicillin-streptomycin.

In Vitro RNA Expression (qRT-PCR)

In vitro mRNA expression of the plasmid was demonstrated by transfection of COS-7 with serially diluted plasmids followed by analysis of the total RNA extracted from the cells using reverse transcription and PCR. Transfections of four concentrations of the plasmid were performed using FuGENE® 6 transfection reagent (Promega) which resulted in final masses ranging between 80 and 10 ng per well. The transfections were performed in duplicate. Following 18 to 26 hours of incubation the cells were lysed with RLT Buffer (Qiagen). Total RNA was isolated from each well using the Qiagen RNeasy kit following the kit instructions. The resulting RNA concentration was determined by OD_{260/280} and samples of the RNA were diluted to 10 ng per μL. One hundred nanograms of RNA was then converted to cDNA using the High Capacity cDNA Reverse Transcription (RevT) kit (Applied Biosystems) following the kit instructions. RevT reactions containing RNA but no reverse transcriptase (minus RT) were included as controls for plasmid DNA or cellular genomic DNA sample contamination. Eight μL of sample cDNA were then subjected to PCR using primers and probes that are specific to the target sequence. In a separate reaction, the same quantity of sample cDNA was subjected to PCR using primers and probe designed for COS-7 cell line β-actin sequences. Using a QuantStudio 7 Flex Real Time PCR Studio System (Applied Biosystems), samples were first subjected to a hold of 1 minute at 95° C. and then 40 cycles of PCR with each cycle consisting of 1 second at 95° C. and 20 seconds at 60° C. Following PCR, the amplifications results were analyzed as follows. The negative transfection controls, the minus RevT controls, and the NTC were scrutinized for each of their respective indications. The threshold cycle (C_{T}) of each transfection concentration for the INO-4800 COVID-19 target mRNA and for the β-actin mRNA was generated from the QuantStudio software using an automatic threshold setting. The plasmid was considered to be active for mRNA expression if the expression in any of the plasmid transfected wells compared to the negative transfection controls were greater than 5 C_{T}.

In Vitro Protein Expression (Western Blot)

Human embryonic kidney cells, 293T were cultured and transfected as described previously (Yan et al., 2007, Mol Ther, 15:411-421). 293T cells were transfected with pDNA using TurboFectin8.0 (OriGene) transfection reagent following the manufacturer's protocol. Forty-eight hours later cell lysates were harvested using modified RIPA cell lysis buffer. Proteins were separated on a 4-12% BIS-TRIS gel (ThermoFisher Scientific), then following transfer, blots were incubated with an anti-SARS-CoV spike protein polyclonal antibody (Novus Biologicals) then visualized with horseradish peroxidase (HRP)-conjugated anti-mouse IgG (GE Amersham).

Immunofluorescence of Transfected 293T Cells

For in vitro staining of Spike protein expression 293T cells were cultured on 4-well glass slides (Lab-Tek) and transfected with 3 μg per well of pDNA using TurboFectin8.0 (OriGene) transfection reagent following the manufacturer's protocol. Cells were fixed 48 hrs after transfection with 10% Neutral-buffered Formalin (BBC Biochemical, Washington State) for 10 min at room temperature (RT) and then washed with PBS. Before staining, chamber slides were blocked with 0.3% (v/v) Triton-X (Sigma), 2% (v/v) donkey serum in PBS for 1 hr at RT. Cells were stained with a rabbit anti-SARS-CoV spike protein polyclonal antibody (Novus Biologicals) diluted in 1% (w/v) BSA (Sigma), 2% (v/v) donkey serum, 0.3% (v/v) Triton-X (Sigma) and 0.025% (v/v) 1 g ml⁻¹ Sodium Azide (Sigma) in PBS for 2 hrs at RT. Slides were washed three times for 5 min in PBS and then stained with donkey anti-rabbit IgG AF488 (lifetechnologies) for 1 hr at RT. Slides were washed again and mounted and covered with DAPI-Fluoromount (SouthemBiotech).

Animals

Female, 6 week old C57/BL6 and BALB/c mice were purchased from Charles River Laboratories (Malvern, Pa.) and The Jackson Laboratory (Bar Harbor, Me.). Female, 8 week old Hartley guinea pigs were purchased from Elm Hill Labs (Chelmsford, Mass.). For mouse studies, on day 0 doses of 2.5, 10 or 25 μg pDNA were administered to the tibialis anterior (TA) muscle by needle injection followed by CELLECTRA® in vivo electroporation (EP). The CELLECTRA® EP delivery consists of two sets of pulses with 0.2 Amp constant current. Second pulse sets is delayed 3 seconds. Within each set there are two 52 ms pulses with a 198 ms delay between the pulses. On days 0 and 14 blood was collected. Parallel groups of mice were serially sacrificed on days 4, 7, and 10 post-immunization for analysis of cellular immune responses. For guinea pig studies, on day 0, 100 μg pDNA was administered to the skin by Mantoux injection followed by CELLECTRA® in vivo EP. Blood was collected on day 0 and 14. For ACE2 competition ELISAs and lung bronchoalveolar lavage, mice and guinea pigs were immunized twice at days 0 and 14. Sera was collected 14 days post-2^{nd} immunization for mice and 28 days post-2^{nd} immunization for guinea pigs.

Antigen Binding ELISA

ELISAs were performed to determine sera antibody binding titers. Nunc ELISA plates were coated with 1 μg ml⁻¹ recombinant protein antigens in Dulbecco's phosphate-buffered saline (DPBS) overnight at 4° C. Plates were washed three times then blocked with 3% bovine serum albumin (BSA) in DPBS with 0.05% Tween 20 for 2 hours at 37° C. Plates were then washed and incubated with serial dilutions of mouse or guinea pig sera and incubated for 2 hours at 37° C. Plates were again washed and then incubated with 1:10,000 dilution of horse radish peroxidase (HRP) conjugated anti-guinea pig IgG secondary antibody (Sigma-Aldrich, cat. A7289) or (HRP) conjugated anti-mouse IgG secondary antibody (Sigma-Aldrich) and incubated for 1 hour at RT. After final wash plates were developed using SureBlue™ TMB 1-Component Peroxidase Substrate (KPL, cat. 52-00-03) and the reaction stopped with TMB Stop Solution (KPL, cat. 50-85-06). Plates were read at 450 nm wavelength within 30 minutes using a Synergy HTX (BioTek Instruments, Highland Park, Vt.). Binding antibody endpoint titers (EPTs) were calculated as previously described in Bagarazzi M et al. 2012 (Bagarazzi et al., 2012, Sci Transl Med, 4:155ra138). Binding antigens tested included, SARS-CoV-2 antigens: S1 spike protein (Sino Biological 40591-V08H), S1+S2 ECD spike protein (Sino Biological 40589-V08B1), RBD (University of Texas, at Austin (McLellan Lab.)); SARS-COV antigens: Spike S1 protein (Sino Biological 40150-V08B1), S (1-1190) (Immune Tech IT-002-001P) and Spike C-terminal (Meridian Life Science R18572).

ACE-2 Competition ELISA

Mice

ELISAs were performed to determine sera IgG antibody competition against human ACE2 with a human Fc tag. Nunc ELISA plates were coated with 1 μg/ml rabbit anti-His6× in 1×PBS for 4-6 hours at room temperature and washed 4 times with washing buffer (1×PBS and 0.05% Tween 20). Plates were blocked overnight at 4° C. with blocking buffer (1×PBS, 0.05% Tween 20, 5% evaporated milk and 1% FBS). Plates were washed four times with washing buffer then incubated with full length (S1+S2) spike protein containing a C-terminal His tag (Sino Biologics, cat. 40589-V08B1) at 10 ug/ml for 1 hour at room temperature. Plates were washed and then serial dilutions of purified mouse IgG mixed with 0.1 ug/ml recombinant human ACE2 with a human Fc tag (ACE2-IgHu) were incubated for 1-2 hours at room temperature. Plates were again washed and then incubated with 1:10,000 dilution of horse radish peroxidase (HRP) conjugated anti-human IgG secondary antibody (Bethyl, cat. A80-304P) and incubated for 1 hour at room temperature. After final wash plates were developed using 1-Step Ultra TMB-ELISA Substrate (Thermo, cat. 34029) and the reaction stopped with 1 M Sulfuric Acid. Plates were read at 450 nm wavelength within 30 minutes using a SpectraMax Plus 384 Microplate Reader (Molecular Devices, Sunnyvale, Calif.). Competition curves were plotted and the area under the curve (AUC) was calculated using Prism 8 analysis software with multiple t-tests to determine statistical significance.

Guinea Pigs

96 well half area assay plates (Costar) were coated with 25 μl/well of 5 μg/mL of SARS-CoV-2 spike S1+S2 protein (Sino Biological) diluted in 1×DPBS (Thermofisher) overnight at 4° C. Plates were washed with 1×PBS buffer with 0.05% TWEEN (Sigma). 100 μl/well of 3% (w/v) BSA (Sigma) in 1×PBS with 0.05% TWEEN were added and incubated for 1 hr at 37° C. Serum samples were diluted 1:20 in 1% (w/v) BSA in 1×PBS with 0.05% TWEEN. After washing the assay plate, 25 μl/well of diluted serum was added and incubated 1 hr at 37° C. Human recombinant ACE-2-Fc-tag (Sinobiological) was added directly to the diluted serum, followed by 1 hr of incubation at 37° C. Plates were washed and 25 μl/well of 1:10,000 diluted goat anti-hu Fc fragment antibody HRP (bethyl) was added to the assay plate. Plates were incubated 1 hr at room temperature. For development the SureBlue/TMB Stop Solution (KPL, MD) was used and O.D. was recorded at 450 nm.

Bronchoalveolar Lavage Collection

Bronchoalveolar lavage (BAL) fluid was collected by washing the lungs of euthanized and exsanguinated mice with 700-1000 ul of ice-cold PBS containing 100 μm EDTA, 0.05% sodium azide, 0.05% Tween-20, and 1× protease inhibitor (Pierce) (mucosal prep solutions (MPS) with a blunt-ended needle. Guinea pig lungs were washed with 20 ml of MPS via 16 G catheter inserted into the trachea. Collected BAL fluid was stored at −20 C until the time of assay.

IFN-γ ELISpot

Spleens from mice were collected individually in RPMI1640 media supplemented with 10% FBS (R10) and penicillin/streptomycin and processed into single cell suspensions. Cell pellets were re-suspended in 5 mL of ACK lysis buffer (Life Technologies, Carlsbad, Calif.) for 5 min RT, and PBS was then added to stop the reaction. The samples were again centrifuged at 1,500 g for 10 min, cell pellets re-suspended in R10, and then passed through a 45 μm nylon filter before use in ELISpot assay. ELISpot assays were performed using the Mouse IFN-γ ELISpot^{PLUS} plates (MABTECH). 96-well ELISpot plates pre-coated with capture antibody were blocked with R10 medium overnight at 4° C. 200,000 mouse splenocytes were plated into each well and stimulated for 20 hours with pools of 15-mer peptides overlapping by 9 amino acid from the SARS-CoV-2, SARS-CoV, or MERS-CoV Spike proteins (5 peptide pools per protein). Additionally, matrix mapping was performed using peptide pools in a matrix designed to identify immunodominant responses. Cells were stimulated with a final concentration of 5 μL of each peptide per well in RPMI+10% FBS (R10). The spots were developed based on manufacturer's instructions. R10 and cell stimulation cocktails (Invitrogen) were used for negative and positive controls, respectively. Spots were scanned and quantified by ImmunoSpot CTL reader. Spot-forming unit (SFU) per million cells was calculated by subtracting the negative control wells.

Flow Cytometry

Intracellular cytokine staining was performed on splenocytes harvested from BALB/c and C57BL/6 mice stimulated with the overlapping peptides spanning the SARS-CoV-2 S protein for 6 hours at 37° C., 5% CO2. Cells were stained with the following antibodies: FITC anti-mouse CD107a, PerCP-Cy5.5 anti-mouse CD4 (BD Biosciences), APC anti-mouse CD8a (BD Biosciences), ViViD Dye (LIVE/DEAD® Fixable Violet Dead Cell Stain kit; Invitrogen, L34955), APC-Cy7 anti-mouse CD3e (BD Biosciences), and BV605 anti-mouse IFN-γ (eBiosciences). Phorbol Myristate Acetate (PMA) were used as a positive control, and complete medium only as the negative control. Cells were washed, fixed and, cell events were acquired using an FACS CANTO (BD Biosciences), followed by FlowJo software (FlowJo LLC, Ashland, Oreg.) analysis.

Structural Modeling

The structural models for SARS-CoV and MERS-CoV were constructed from PDB IDs 6acc and 5×59 in order to assemble a prefusion model with all three RBDs in the down conformation. The SARS-CoV-2 structural model was built by using SARS-CoV structure (PDB id:6acc) as a template. Rosetta remodel simulations were employed to make the appropriate amino acid mutations and to build de novo models for SARS-CoV-2 loops not structurally defined in the SARS-CoV structure (Huang et al., 2011, PLoS One, 6:e24109). Amino acid positions neighboring the loops were allowed to change backbone conformation to accommodate the new loops. The structural figures were made using PyMOL.

Statistics

All statistical analyses were performed using GraphPad Prism 7 or 8 software (La Jolla, Calif.). These data were considered significant if p<0.05. The lines in all graphs represent the mean value and error bars represent the standard deviation. No samples or animals were excluded from the analysis. Randomization was not performed for the animal studies. Samples and animals were not blinded before performing each experiment.

The experimental results are now described

Design and Synthesis COVID-19 Synthetic DNA Vaccine Constructs

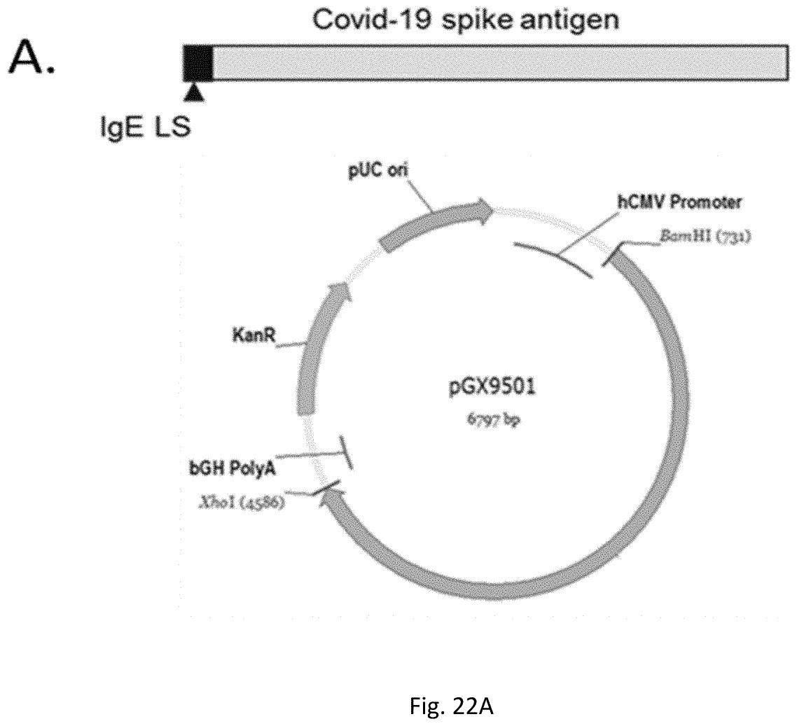

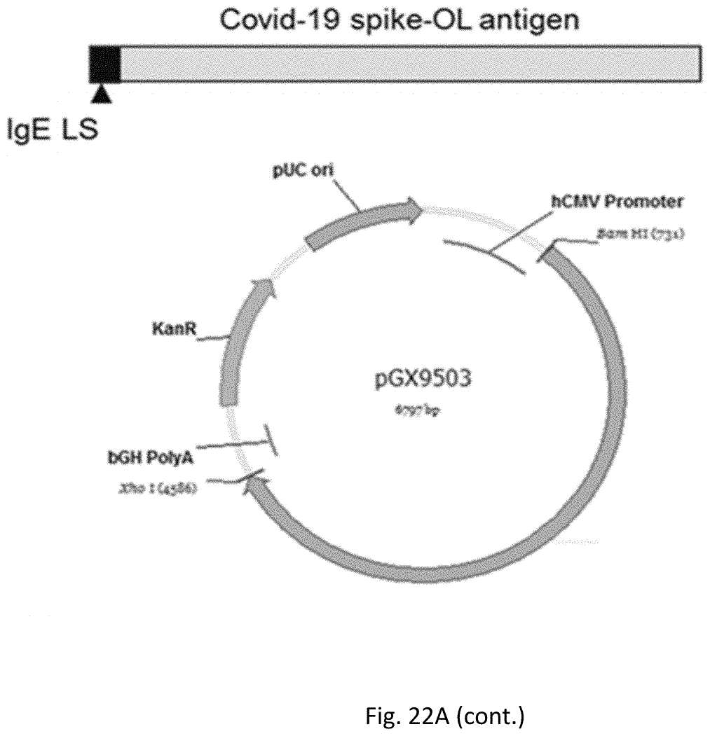

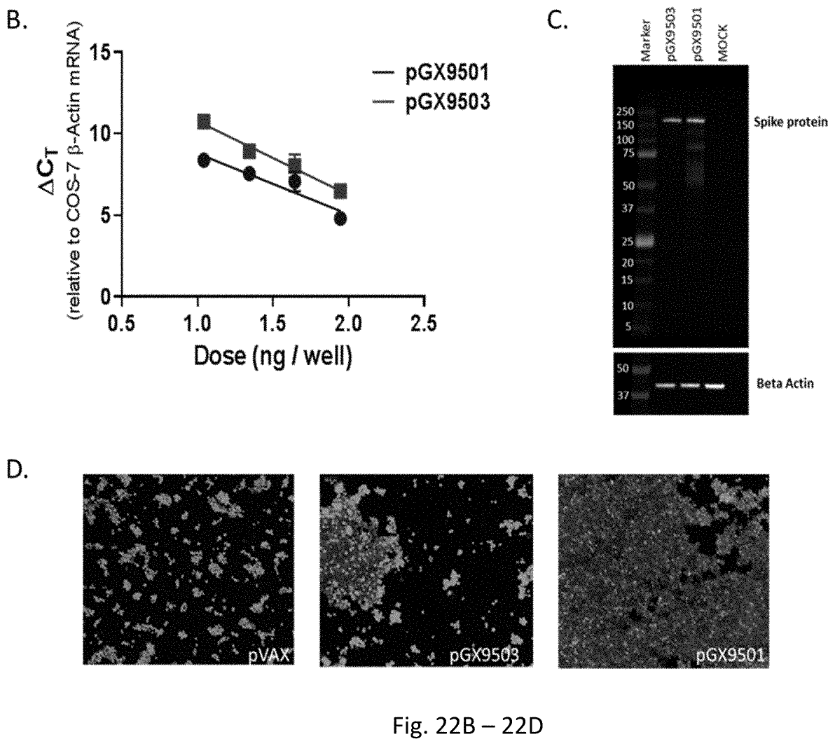

Four spike protein sequences were retrieved from the first four available SARS-CoV-2 full genome sequences published on GISAID (Global Initiative on Sharing All Influenza Data). Three Spike sequences were 100% matched and one was considered an outlier (98.6% sequence identity with the other sequences). After performing a sequence alignment, the SARS-CoV-2 spike glycoprotein sequence was generated and an N-terminal IgE leader sequence was added. The highly optimized DNA sequence encoding SARS-CoV-2 IgE-spike was created using Inovio's proprietary in silico Gene Optimization Algorithm to enhance expression and immunogenicity. The optimized DNA sequence was synthesized, digested with BamHI and XhoI, and cloned into the expression vector pGX0001 under the control of the human cytomegalovirus immediate-early promoter and a bovine growth hormone polyadenylation signal. The resulting plasmids were designated as pGX9501 and pGX9503, designed to encode the SARS-CoV-2 S protein from the 3 matched sequences and the outlier sequence, respectively (FIG. 22A).

In Vitro Characterization of COVID-19 Synthetic DNA Vaccine Constructs

The expression of the encoded SARS-CoV-2 spike transgene was measured at the RNA level in COS-7 cells transfected with pGX9501 and pGX9503. Using the total RNA extracted from the transfected COS-7 cells expression of the spike transgene by RT-PCR was confirmed (FIG. 22B). In vitro spike protein expression in HEK-293T cells was measured by Western blot analysis using a cross-reactive antibody against SARS-CoV S protein on cell lysates. Western blots of the lysates of HEK-293T cells transfected with pGX9501 or pGX9503 constructs revealed bands approximate to the predicted S protein molecular weight, 140-142 kDa (FIG. 22C). In immunofluorescent studies the S protein was detected in 293T cells transfected with pGX9501 or pGX9503 (FIG. 22D). In summary, in vitro studies revealed the expression of the Spike protein at both the RNA and protein level after transfection of cell lines with the candidate vaccine constructs.

Humoral Immune Responses to SARS-CoV-2 S Protein Antigens Measured in Mice Immunized with INO-4800

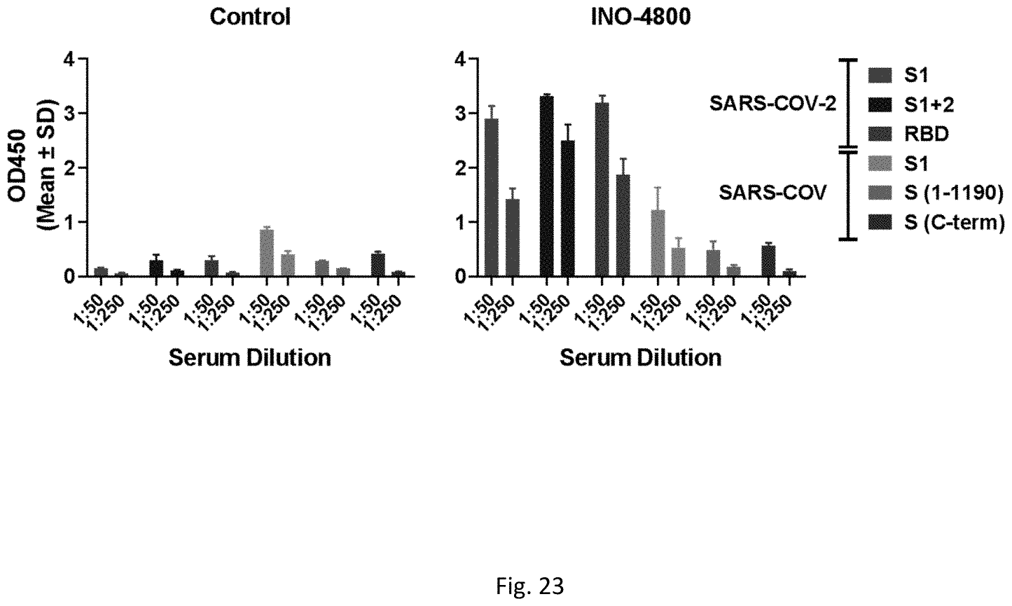

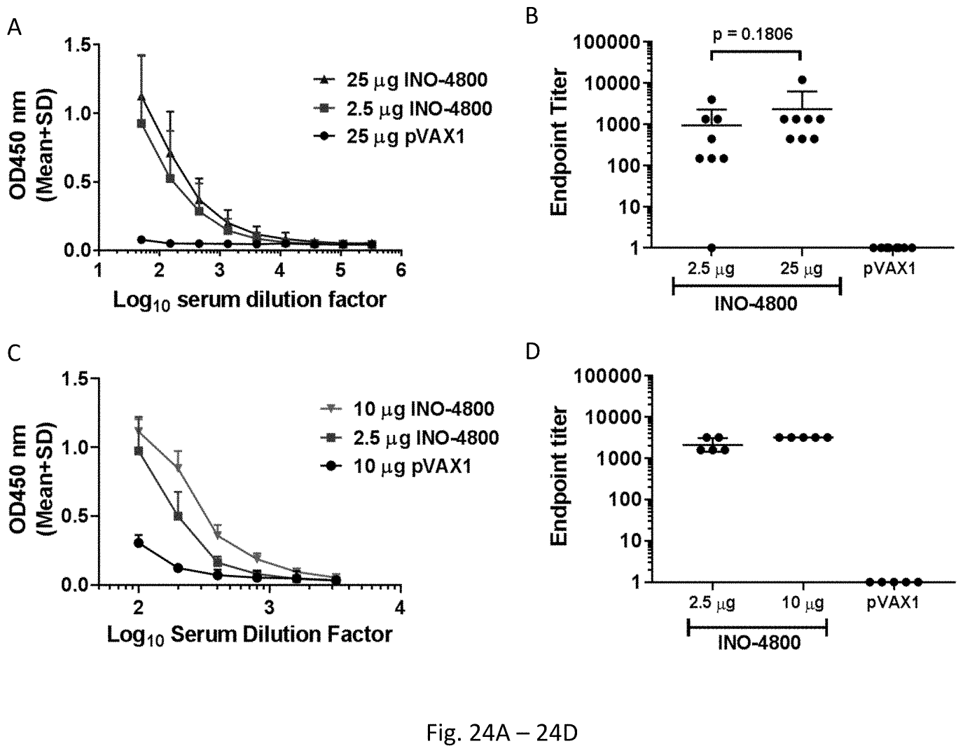

pGX9501 was selected as the vaccine construct to advance to immunogenicity studies, due to the broader coverage it would likely provide compared to the outlier, pGX9503. pGX9501 was subsequently termed INO-4800. The immunogenicity of INO-4800 was evaluated in BALB/c mice, post-administration to the TA muscle followed with CELLECTRA® delivery device¹⁶. The reactivity of the sera from a group of mice immunized with INO-4800 was measured against a panel of SARS-CoV-2 and SARS-CoV antigens (FIG. 23). Analysis revealed IgG binding against SARS-CoV-2 S protein antigens, with limited cross-reactivity to SARS-CoV S protein antigens, in the serum of INO-4800 immunized mice. The serum IgG binding endpoint titers were measured in mice immunized with pDNA against recombinant SARS-CoV-2 spike protein S1+S2 regions (FIG. 24A and FIG. 24B) and recombinant SARS-CoV-2 spike protein receptor binding domain (RBD) (FIG. 24C and FIG. 24D). Endpoint titers were observed in the serum of mice at day 14 after immunization with a single dose of INO-4800 (FIG. 24B-FIG. 24D).

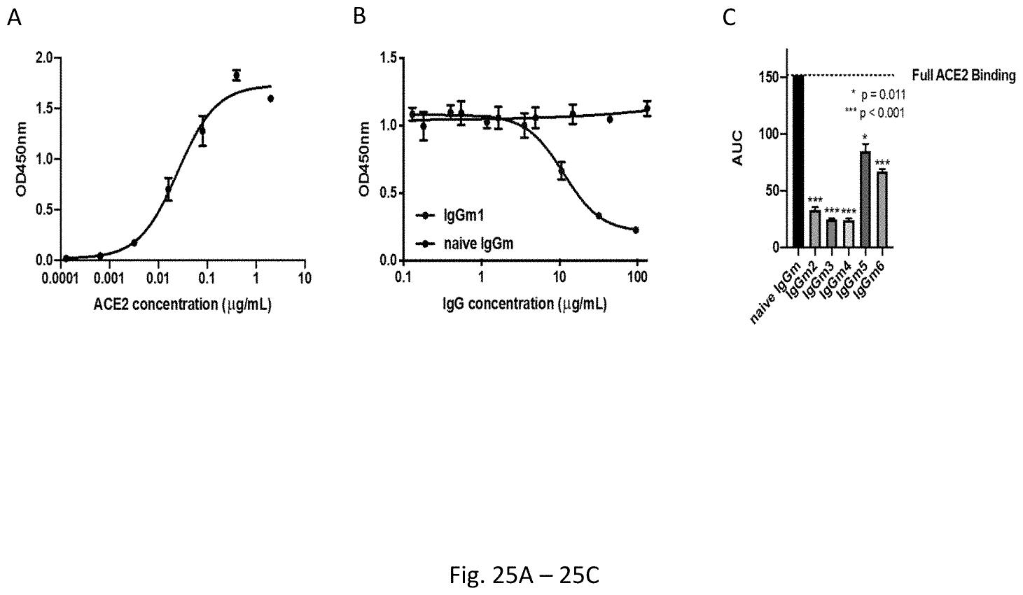

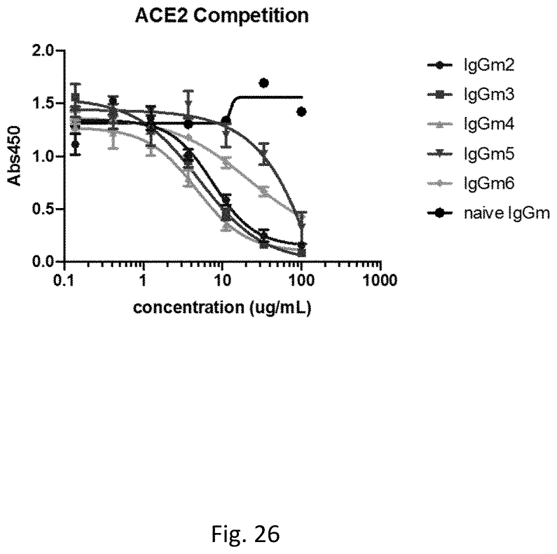

The induction of antibodies capable of inhibiting Spike protein host receptor engagement is a critical goal in SARS-CoV-2 vaccine development. Therefore, experiments were designed to examine the receptor inhibiting functionality of INO-4800-induced antibody responses. Recently an ELISA-based ACE2 inhibition assay was developed as a surrogate for neutralization. The assay is similar in principle to other surrogate neutralization assays which have been validated for coronaviruses (Rosen et al., 2019, J Virol Methods, 265:77-83). As a control in the assay, it was shown that ACE2 can bind to SARS-CoV-2 Spike protein with an EC₅₀ of 0.025 μg/ml (FIG. 25A). BALB/c mice were immunized, on Days 0 and Day 14, with 10 μg of INO-4800, and serum IgG was purified on Day 28 post-immunization to ensure inhibition is antibody mediated. Inhibition of the Spike-ACE2 interaction using serum IgG from a naïve mouse and from an INO-4800 vaccinated mouse were compared (FIG. 25B). The receptor inhibition assay was repeated with a group of five mice immunized, and it was shown that the INO-4800-induced antibodies competed with ACE2 binding to the SARS-CoV-2 Spike protein (FIG. 25C and FIG. 26). ACE2 is considered to be the primary receptor for SARS-CoV-2 cellular entry, blocking this interaction suggests INO-4800-induced antibodies may prevent host infection.

Detection of Humoral Immune Response to SARS-CoV-2 S Protein in Guinea Pigs after Intradermal Delivery of INO-4800

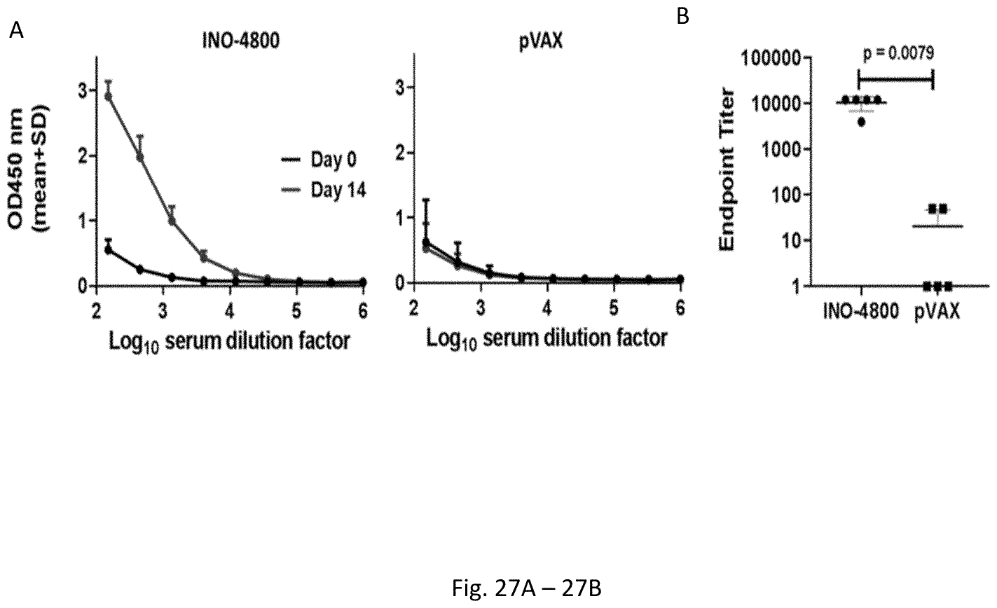

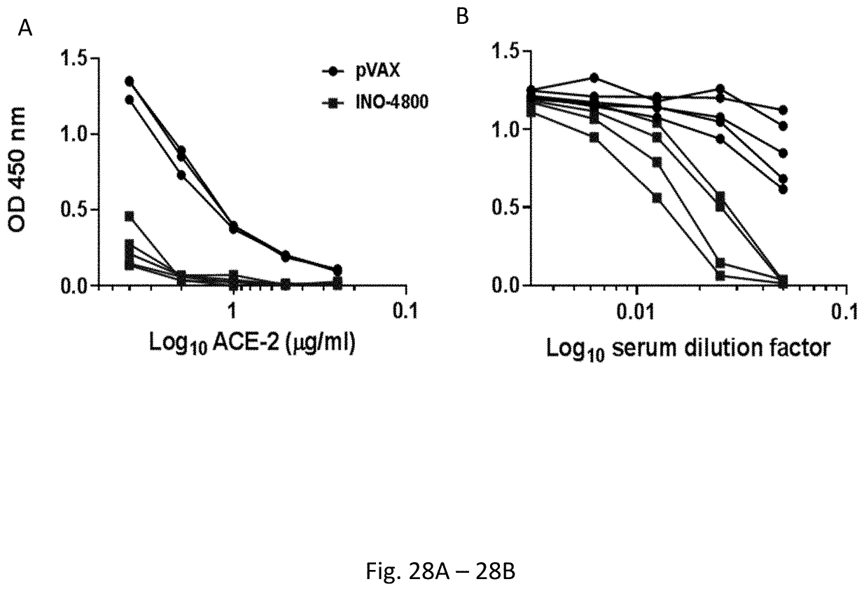

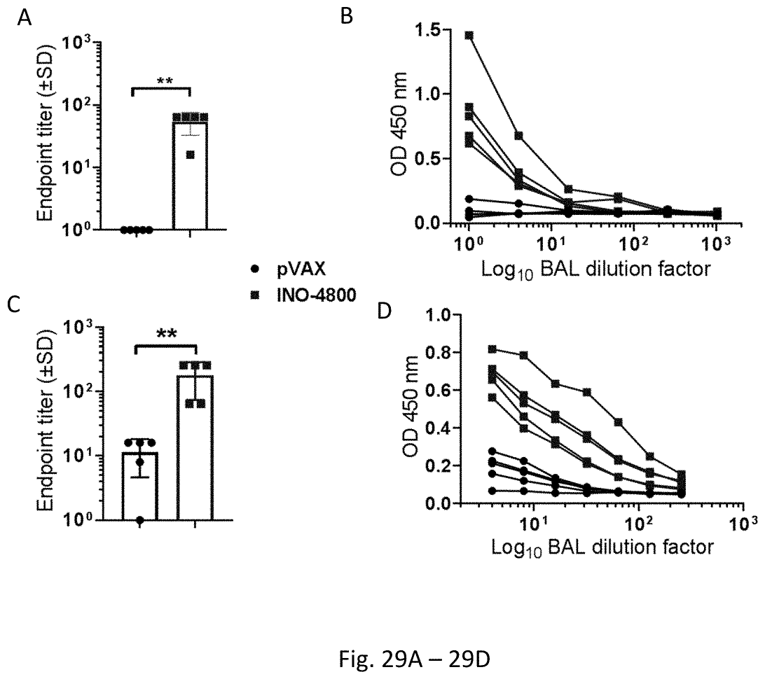

The immunogenicity of INO-4800 was assessed in the Hartley guinea pig model, an established model for intradermal vaccine delivery (Carter et al., 2018, Sci Adv, 4:eaas9930; Schultheis et al., 2017, Vaccine, 35:61-70. 100 μg of pDNA was administered by Mantoux injection to the skin and followed by CELLECTRA® delivery device on day 0 as described in the methods section. On day 14 anti-spike protein binding of serum antibodies was measured by ELISA. Immunization with INO-4800 revealed an immune response in respect to SARS-CoV-2 S1+2 protein binding IgG levels in the serum (FIG. 27A and FIG. 27B). The endpoint SARS-CoV-2 S protein binding titer at day 14 was 10,530 and 21 in guinea pigs treated with 100 μg INO-4800 or pVAX (control), respectively (FIG. 27B). The functionality of the serum antibodies was measured by assessing their ability to inhibit ACE-2 binding to SARS-CoV-2 spike protein. Serum (1:20 dilution) collected from INO-4800 immunized guinea pigs after 2^{nd} immunization inhibited binding of SARS-CoV-2 Spike protein over range of concentrations of ACE-2 (0.25 μg/ml through 4 μg/ml) (FIG. 28A). Furthermore, serum dilution curves revealed serum collected from INO-4800 immunized guinea pigs blocked binding of ACE-2 to SARS-CoV-2 in a dilution-dependent manner (FIG. 28B). Serum collected from pVAX-treated animals displayed negligible activity in the inhibition of ACE-2 binding to the virus protein, the decrease in OD signal at the highest concentration of serum is considered a matrix effect in the assay.

In summary, immunogenicity testing in both mice and guinea pigs revealed the COVID-19 vaccine candidate, INO-4800, was capable of eliciting functional antibody responses to SARS-CoV-2 spike protein.

Biodistribution of SARS-CoV-2 Specific Antibodies to the Lung in INO-4800 Immunized Animals

Lower respiratory disease (LRD) is associated with severe cases of COVID-19. The presence of antibodies at the lung mucosa targeting SARS-CoV-2 could potentially mediate protection against LRD. Therefore, the presence of SARS-CoV-2 specific antibody was evaluated in the lungs of immunized mice and guinea pigs. BALB/c mice and Hartley guinea pigs were immunized, on days 0 and 14 or 0, 14 and 28, respectively, with INO-4800 or pVAX control pDNA. Bronchoalveolar lavage (BAL) fluid was collected following sacrifice, and SARS-CoV-2 S protein ELISAs were performed. In both BALB/c and Hartley guinea pigs which received INO-4800 a statistically significant increased SARS-CoV-2 S protein binding IgG was measured in their BAL fluid compared to animals receiving pVAX control (FIG. 29A through FIG. 29D). Taken together, these data demonstrate the presence of anti-SARS-CoV-2 specific antibody in the lungs following immunization with INO-4800.

Early Detection of Cross-Reactive Cellular Immune Responses Against SARS-CoV-2 and SARS-CoV in Mice Immunized with INO-4800

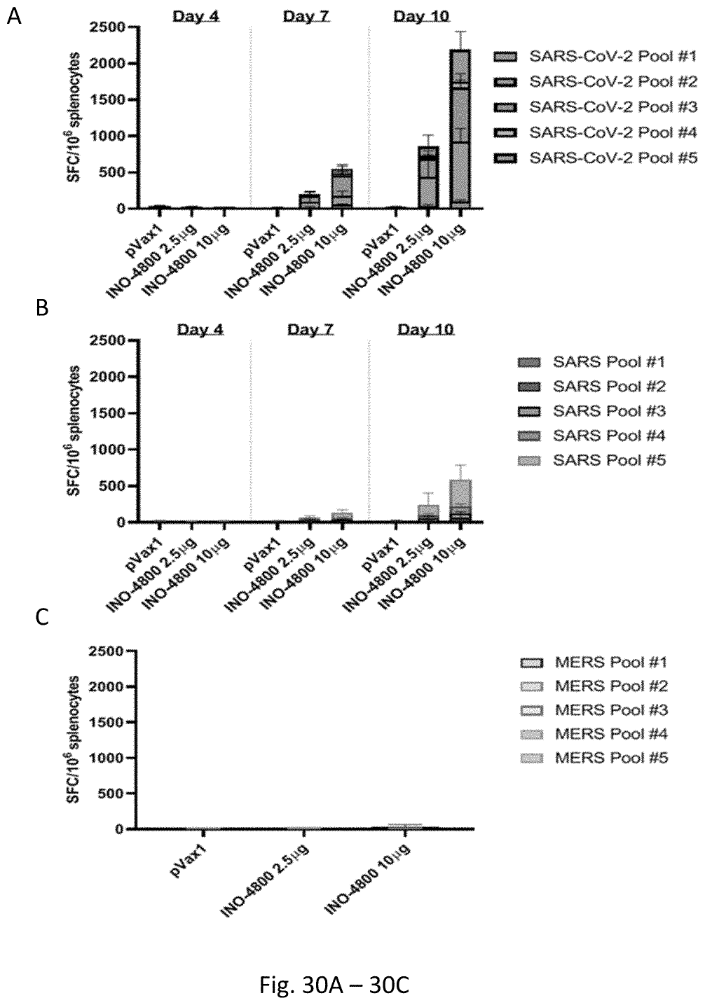

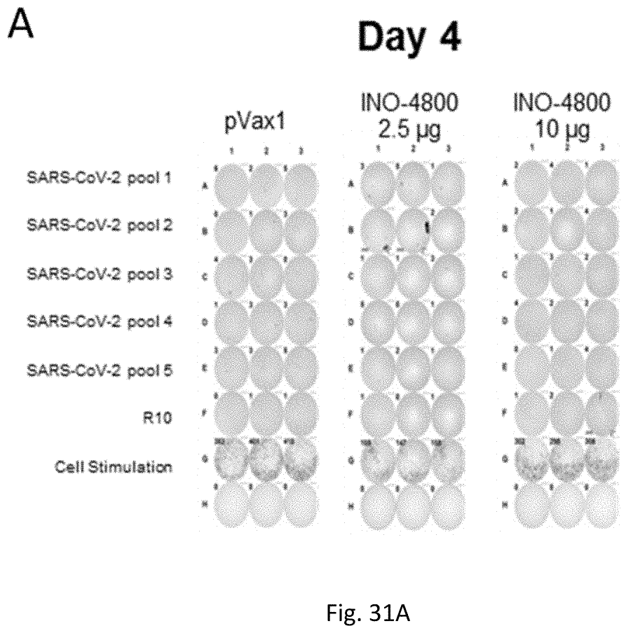

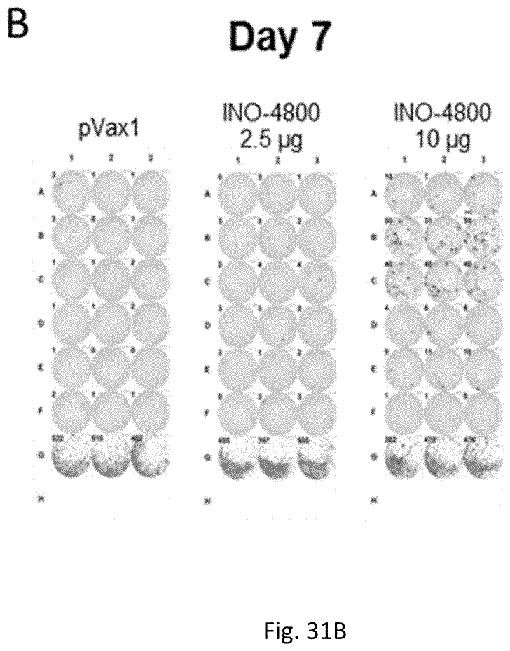

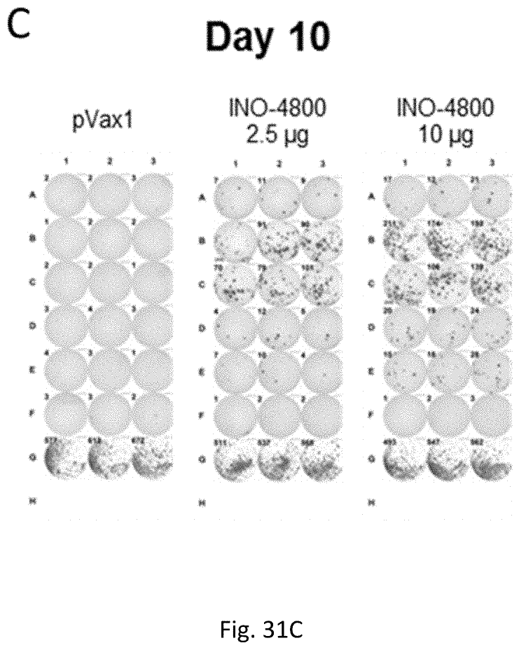

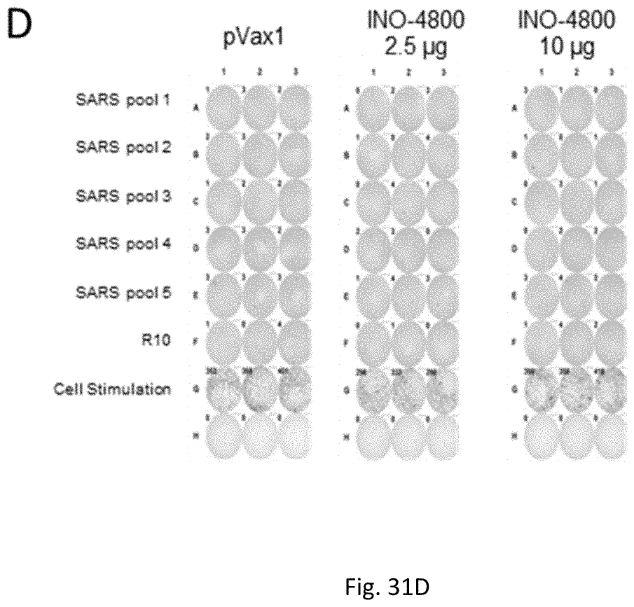

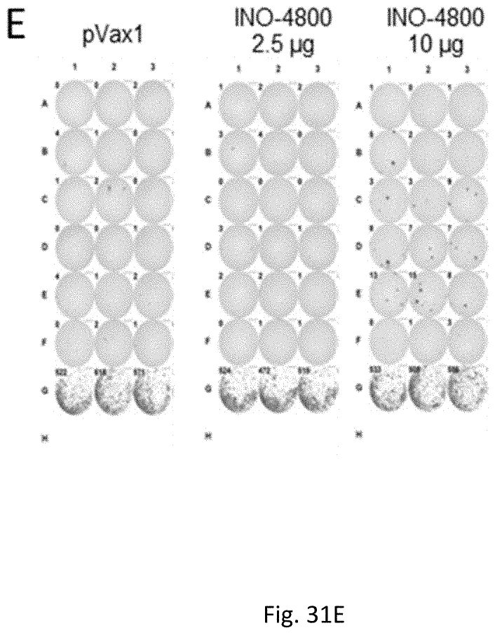

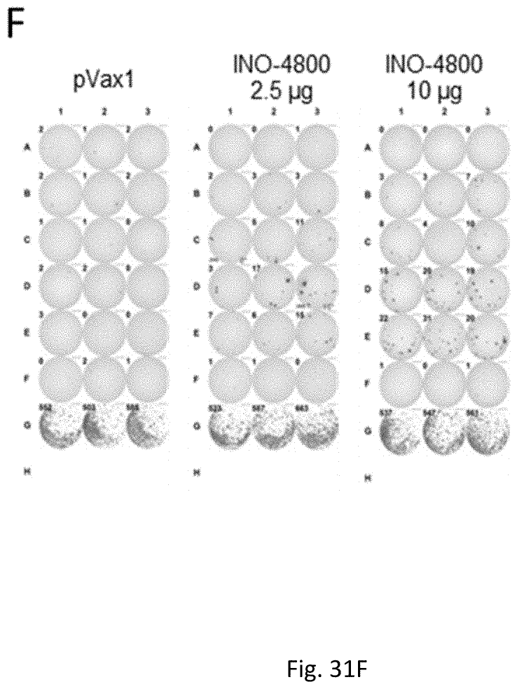

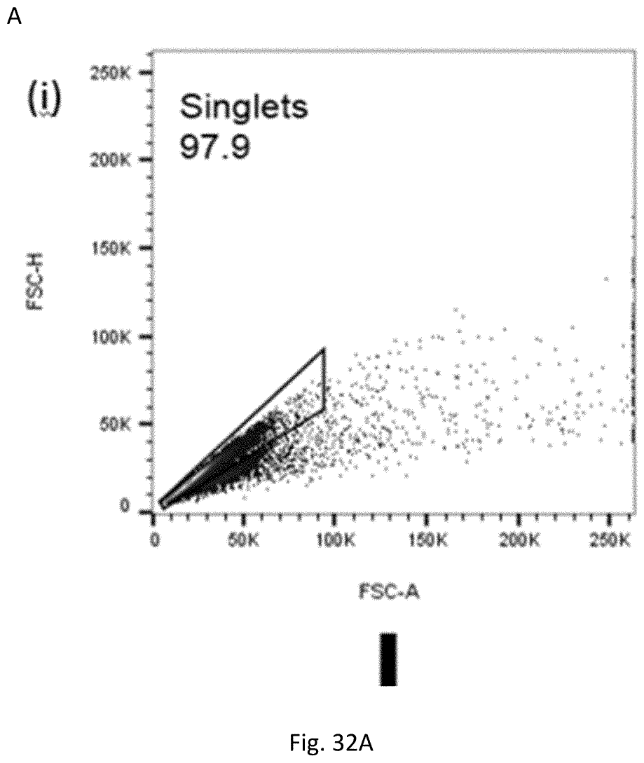

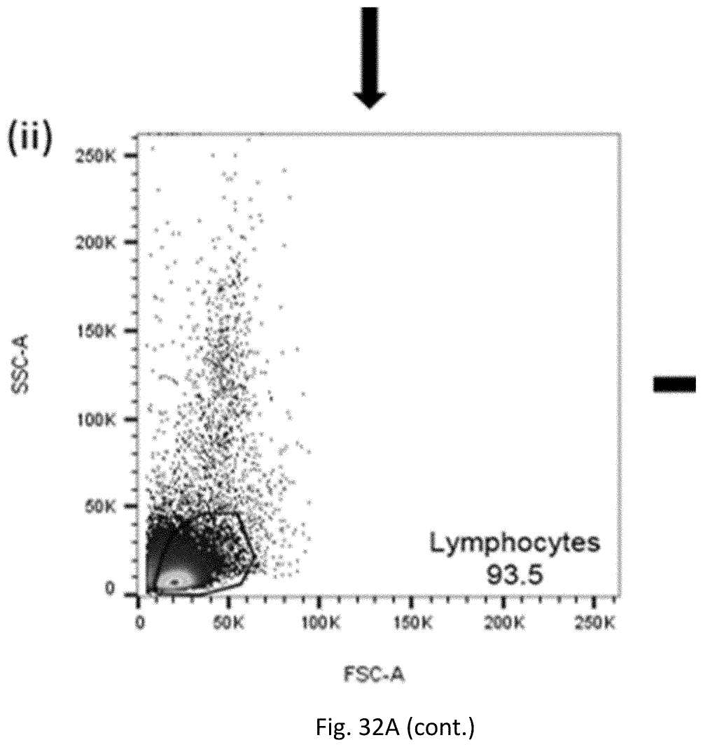

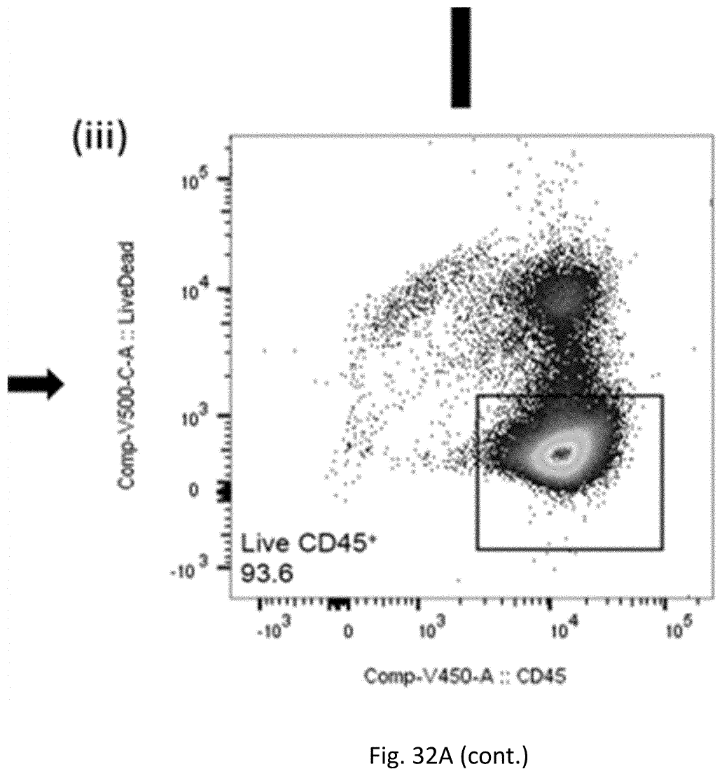

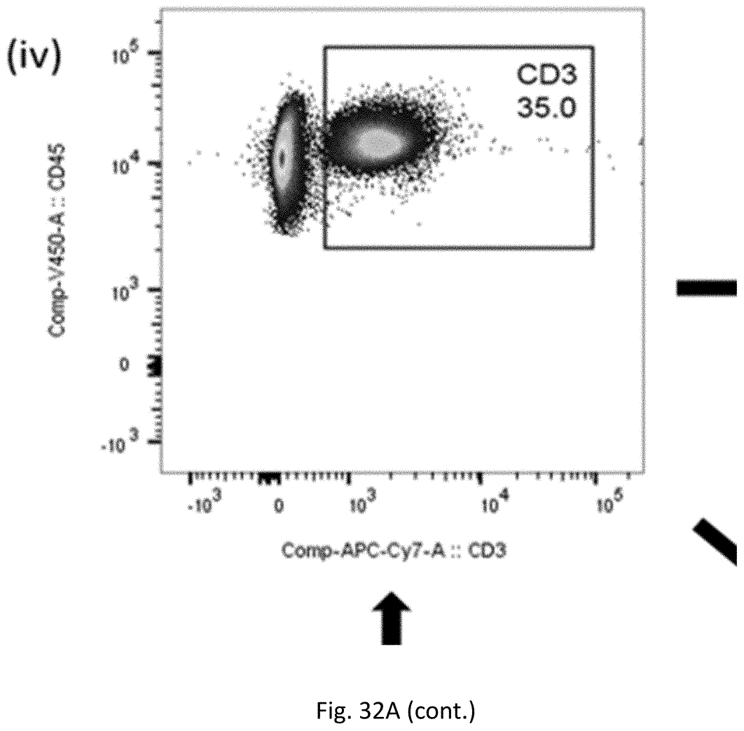

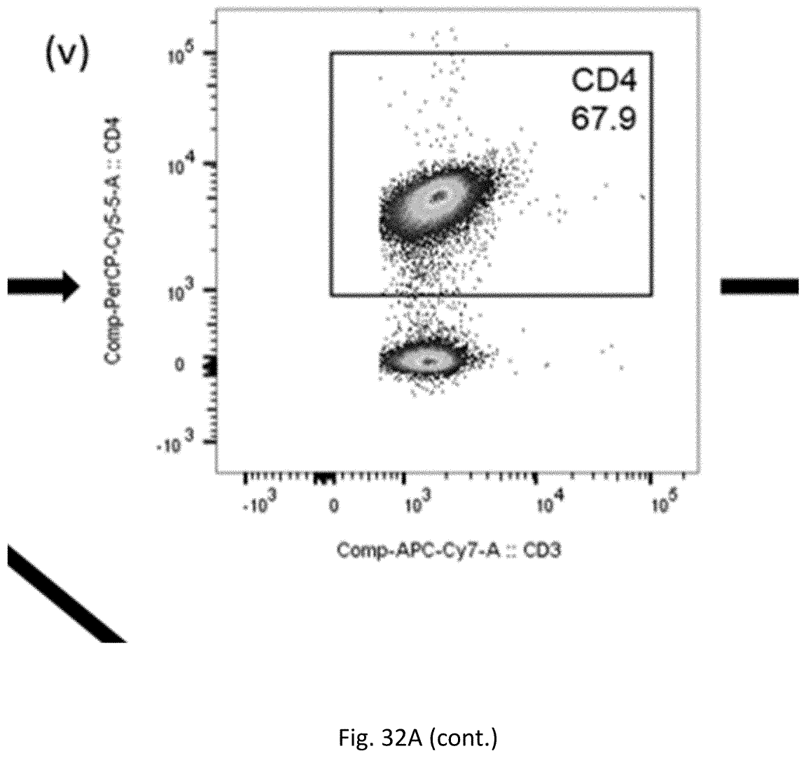

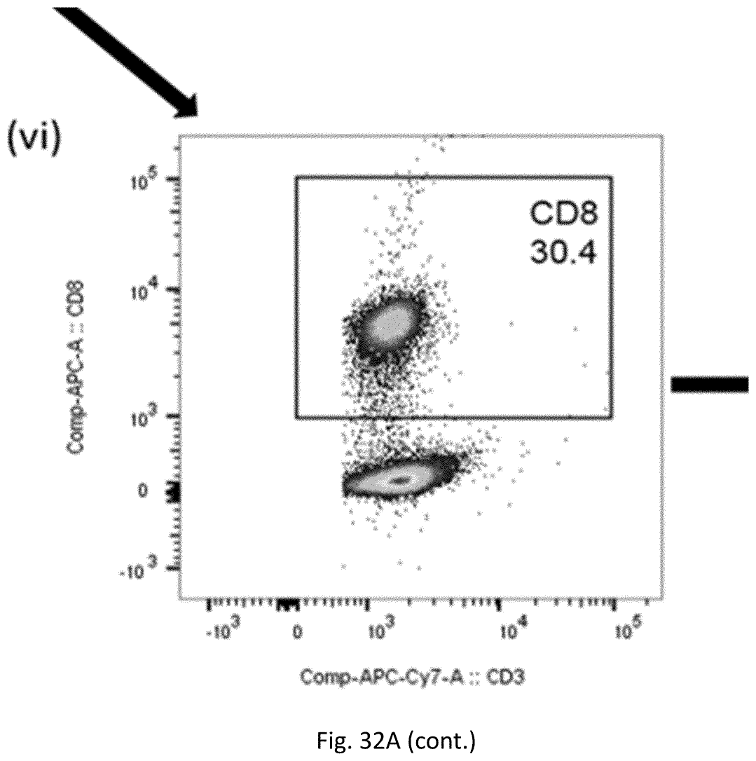

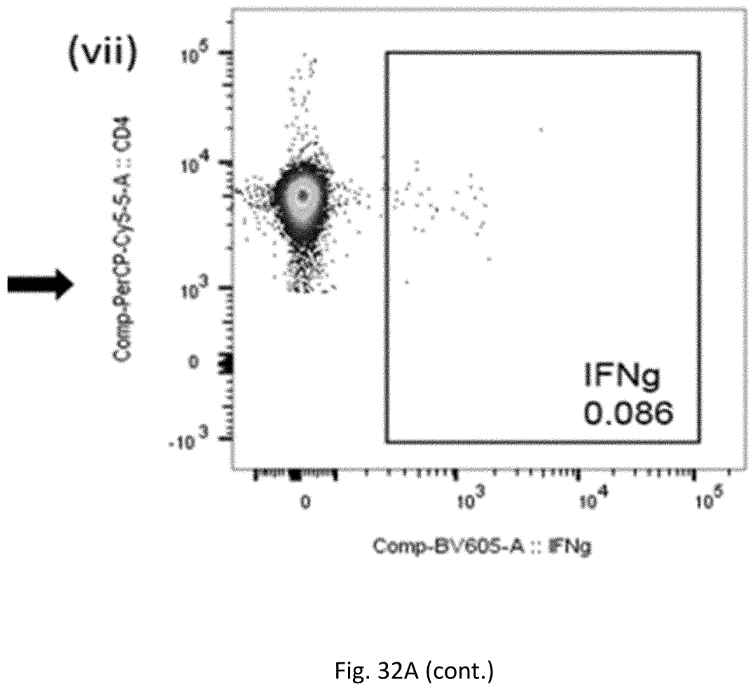

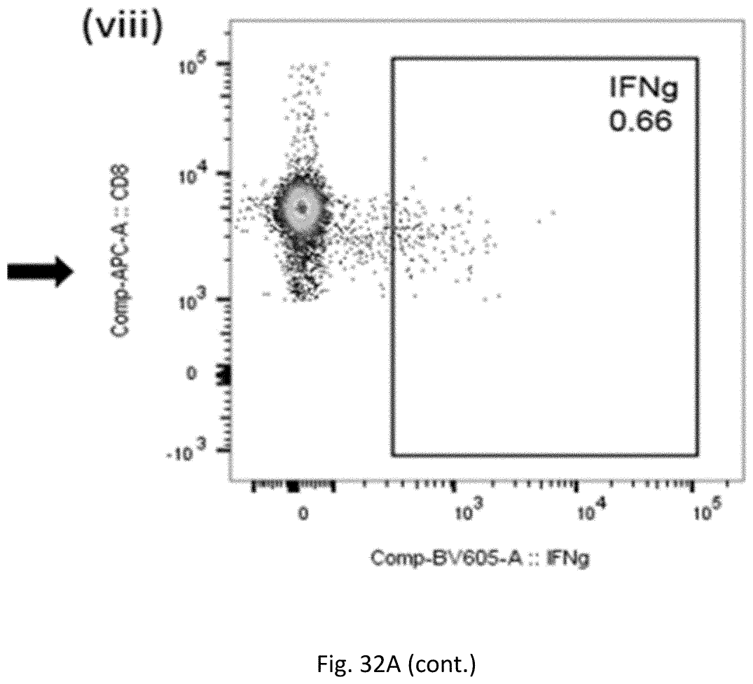

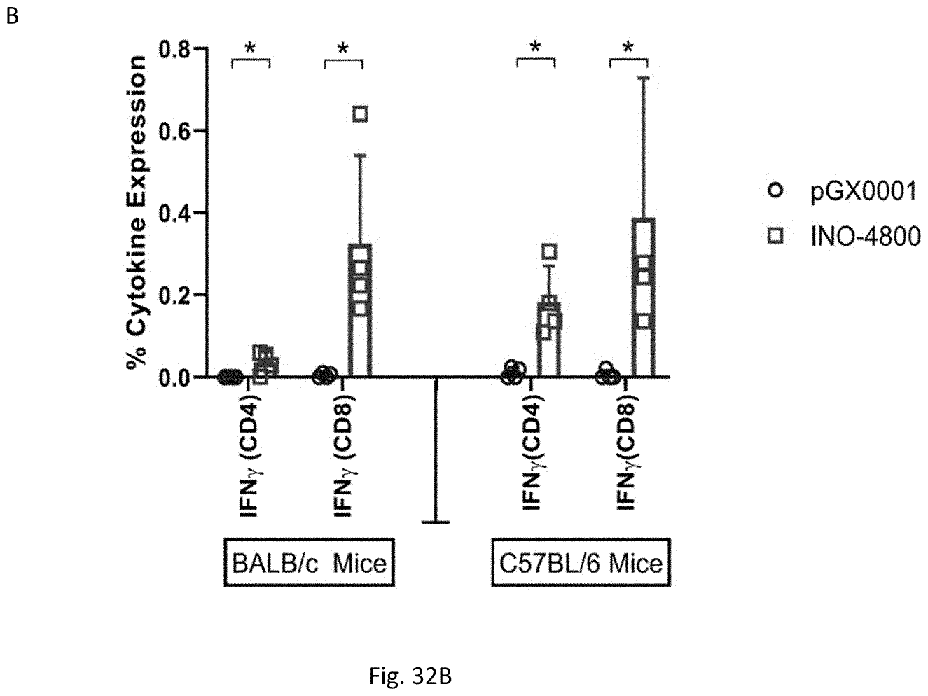

T cell responses against SARS-CoV-2, SARS-CoV, and MERS-CoV S antigens were assayed by IFN-γ ELISpot. Groups of BALB/c mice were sacrificed at days 4, 7, or 10 post-INO-4800 administration (2.5 or 10 μg of pDNA), splenocytes were harvested, and a single-cell suspension was stimulated for 20 hours with pools of 15-mer overlapping peptides spanning the SARS-CoV-2, SARS-CoV, and MERS-CoV spike protein. Day 7 post-INO-4800 administration, T cell responses were measured of 205 and 552 SFU per 10⁶ splenocytes against SARS-CoV-2 for the 2.5 and 10 μg doses, respectively (FIG. 30A). Higher magnitude responses of 852 and 2193 SFU per 10⁶ splenocytes against SARS-CoV-2 were observed on Day 10 post-INO-4800 administration. Additionally, the cross-reactivity of the cellular response elicited by INO-4800 was evaluated against SARS-CoV, and detectable, albeit lower, T cell responses were observed on both Day 7 (74 [2.5 μg dose] and 140 [10 μg dose] SFU per 10⁶ splenocytes) and Day 10 post-administration (242 [2.5 μg dose] and 588 [10 μg dose] SFC per 10⁶ splenocytes) (FIG. 30B). Interestingly, no cross-reactive T cell responses were observed against MERS-CoV peptides (FIG. 30C). Representative images of the IFN-γ ELISpot plates are provided in FIG. 31. The T cell populations which were producing IFN-γ were identified. Flow cytometric analysis on splenocytes harvested from BALB/c mice on Day 14 after a single INO-4800 immunization revealed the T cell compartment to contain 0.0365% CD4+ and 0.3248% CD8+ IFN-γ+ T cells after stimulation with SARS-CoV-2 antigens (FIG. 32).

BALB/c Mouse SARS-CoV-2 Epitope Mapping

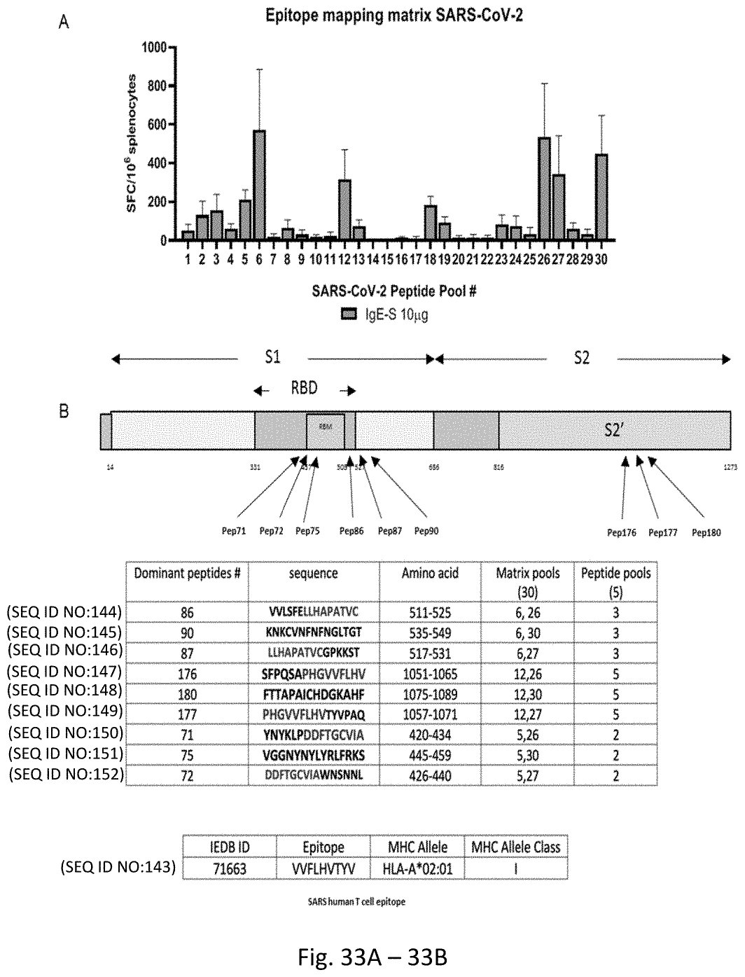

Epitope mapping was performed on the splenocytes from BALB/c mice receiving the 10 μg INO-4800 dose. Thirty matrix mapping pools were used to stimulate splenocytes for 20 hours and immunodominant responses were detected in multiple peptide pools (FIG. 33A). The responses were deconvoluted to identify several epitopes (H2-K^{d}) clustering in the receptor binding domain and in the S2 domain (FIG. 33B). Interestingly, one SARS-CoV-2 H2-K^{d} epitope, PHGVVFLHV (SEQ ID NO:142), was observed to be overlapping and adjacent to the SARS-CoV human HLA-A2 restricted epitope VVFLHVTYV (SEQ ID NO:143) (Ahmed et al., 2020, Viruses 12:254).

In summary, rapid T cell responses against SARS-CoV-2 S protein epitopes were detected in mice immunized with INO-4800.

### Example 6: DMAb and DNA Vaccine Co-Delivery

Studies are designed that demonstrate the efficacy of DMAb+DNA vaccine co-delivery.

### Vaccine Sequences:

| SEQ | | |
|---|---|---|
| ID | | |
| NO: | Sequence Type | Description |
| 133 | DNA | Optimized SARS-CoV-2 Spike antigen |
| 134 | Amino Acid | Optimized SARS-CoV-2 Spike antigen |
| 135 | DNA | Optimized SARS-CoV-2 Spike antigen linked |
| | | to IgE leader |
| 136 | Amino Acid | Optimized SARS-CoV-2 Spike antigen antigen |
| | | linked to IgE leader |
| 137 | DNA | Optimized SARS-CoV-2 outlier Spike antigen |
| 138 | Amino Acid | Optimized SARS-CoV-2 outlier Spike antigen |
| 139 | DNA | Optimized SARS-CoV-2 outlier Spike antigen |
| | | linked to IgE leader |
| 140 | Amino Acid | Optimized SARS-CoV-2 outlier Spike antigen |
| | | antigen linked to IgE leader |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. An anti-SARS-CoV-2 antibody or fragment thereof, wherein the antibody is selected from the group consisting of a monoclonal antibody, a polyclonal antibody, a single chain antibody, an immunoconjugate, a defucosylated antibody, and a bispecific antibody.

2. The anti-SARS-CoV-2 antibody or fragment thereof of claim 1, wherein the immunoconjugate comprises a therapeutic agent or a detection moiety.

3. The anti-SARS-CoV-2 antibody or fragment thereof of claim 1, wherein the antibody is selected from the group consisting of a humanized antibody, a chimeric antibody, a fully human antibody, an antibody mimetic.

4. The anti-SARS-CoV-2 antibody or fragment thereof of claim 1, wherein the antibody comprises at least one selected from the group consisting of:
a) the heavy chain CDR1 sequence selected from the group consisting of SEQ ID NO:26, SEQ ID NO: 80 and SEQ ID NO:108;
b) the heavy chain CDR2 sequence selected from the group consisting of SEQ ID NO:27, SEQ ID NO: 81 and SEQ ID NO:109;
c) the heavy chain CDR3 sequence selected from the group consisting of SEQ ID NO:28, SEQ ID NO: 82 and SEQ ID NO:110;
d) the light chain CDR1 sequence selected from the group consisting of SEQ ID NO:34, SEQ ID NO: 88 and SEQ ID NO:116;
e) the light chain CDR2 sequence selected from the group consisting of SEQ ID NO:35, SEQ ID NO: 89 and SEQ ID NO:117; and
f) the light chain CDR3 sequence selected from the group consisting of SEQ ID NO:36, SEQ ID NO: 90 and SEQ ID NO:118.

5. The anti-SARS-CoV-2 antibody or fragment thereof of claim 1, wherein the antibody comprises at least one selected from the group consisting of:
a) an anti-SARS-CoV-2 antibody heavy chain comprising a sequence having at least 80% identity to an amino acid sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128;
b) an anti-SARS-CoV-2 antibody light chain comprising a sequence having at least 80% identity to an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130;
c) a fragment of an anti-SARS-CoV-2 antibody heavy chain comprising at least 80% of the full length sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and
d) a fragment of an anti-SARS-CoV-2 antibody light chain comprising at least 80% of the full length sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130.

6. The anti-SARS-CoV-2 antibody, or fragment thereof, of claim 1, wherein the antibody comprises at least one selected from the group consisting of:
a) an amino acid sequence having at least 80% identity to an amino acid sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132; and
b) a fragment of an anti-SARS-CoV-2 antibody heavy chain comprising at least 80% of the full length sequence selected from the group consisting of SEQ ID NO:10, SEQ ID NO:16, SEQ ID NO:22, SEQ ID NO:40, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:98, SEQ ID NO:104, SEQ ID NO:126 and SEQ ID NO:132.

7. A nucleic acid molecule encoding an anti-SARS-CoV-2 antibody or fragment thereof of any one of claims 1-6.

8. The nucleic acid molecule of claim 7, further comprising a nucleotide sequence encoding a cleavage domain.

9. The nucleic acid molecule of claim 7, wherein the nucleic acid molecule comprises at least one selected from the group consisting of:
a) a nucleotide sequence selected from the group consisting of SEQ ID NO:23, SEQ ID NO: 77 and SEQ ID NO:105 encoding the heavy chain CDR1 sequence;
b) a nucleotide sequence selected from the group consisting of SEQ ID NO:24, SEQ ID NO: 78 and SEQ ID NO:106 encoding the heavy chain CDR2 sequence;
c) a nucleotide sequence selected from the group consisting of SEQ ID NO:25, SEQ ID NO: 79 and SEQ ID NO:107 encoding the heavy chain CDR3 sequence;
d) a nucleotide sequence selected from the group consisting of SEQ ID NO:31, SEQ ID NO: 85 and SEQ ID NO:113 encoding the light chain CDR1 sequence;
e) a nucleotide sequence selected from the group consisting of SEQ ID NO:32, SEQ ID NO: 86 and SEQ ID NO:114 encoding the light chain CDR2 sequence; and
f) a nucleotide sequence selected from the group consisting of SEQ ID NO:33, SEQ ID NO: 87 and SEQ ID NO:115 encoding the light chain CDR3 sequence.

10. The nucleic acid molecule of claim 7, wherein the nucleic acid molecule comprises at least one nucleotide sequence selected from the group consisting of:
a) a nucleotide sequence having at least 80% identity to a nucleotide selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain;
b) a nucleotide sequence having at least 80% identity to a nucleotide selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain;
c) a fragment of a nucleotide sequence comprising at least 80% of the full length sequence selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and
d) a fragment of a nucleotide sequence comprising at least 80% of the full length sequence selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain.

11. The nucleic acid molecule of claim 7, wherein the nucleic acid molecule comprises a nucleotide sequence selected from the group consisting of:
a) a nucleotide sequence having at least 80% identity to a nucleotide selected from the group consisting of SEQ ID NO:9, SEQ ID NO:15, SEQ ID NO:21, SEQ ID NO:39, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:97, SEQ ID NO:103, SEQ ID NO:125 and SEQ ID NO:131; and
b) a fragment of a nucleotide sequence comprising at least 80% of the full length sequence selected from the group consisting of SEQ ID NO:9, SEQ ID NO:15, SEQ ID NO:21, SEQ ID NO:39, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:97, SEQ ID NO:103, SEQ ID NO:125 and SEQ ID NO:131.

12. The nucleic acid molecule of any one of claims 7-11, wherein the nucleotide sequence encodes a leader sequence.

13. The nucleic acid molecule of any one of claims 7-11, wherein the nucleic acid molecule comprises an expression vector.

14. A composition comprising at least one anti-SARS-CoV-2 antibody or fragment thereof of any one of claims 1-6.

15. A composition comprising at least one nucleic acid molecule of any one of claims 7-11.

16. The composition of claim 15 comprising a first nucleic acid molecule comprising a nucleotide sequence encoding a heavy chain of an anti-SARS-CoV-2 spike antigen synthetic antibody; and a second nucleic acid molecule comprising a nucleotide sequence encoding a light chain of an anti-SARS-CoV-2 spike antigen synthetic antibody.

17. The composition of claim 16, wherein the first nucleic acid molecule comprises a nucleotide sequence encoding at least one selected from the group consisting of:
a) an anti-SARS-CoV-2 antibody heavy chain comprising a sequence having at least 80% identity to an amino acid sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and
b) a fragment of an anti-SARS-CoV-2 antibody heavy chain comprising at least 80% of the full length sequence selected from the group consisting of SEQ ID NO:2, SEQ ID NO:6, SEQ ID NO:12, SEQ ID NO:18, SEQ ID NO:30, SEQ ID NO:42, SEQ ID NO:84, SEQ ID NO:94, SEQ ID NO:100, SEQ ID NO:112, SEQ ID NO:122 and SEQ ID NO:128; and
wherein the second nucleic acid molecule comprises a nucleotide sequence encoding at least one selected from the group consisting of:
c) an anti-SARS-CoV-2 antibody light chain comprising a sequence having at least 80% identity to an amino acid sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130; and
d) a fragment of an anti-SARS-CoV-2 antibody light chain comprising at least 80% of the full length sequence selected from the group consisting of SEQ ID NO:4, SEQ ID NO:8, SEQ ID NO:14, SEQ ID NO:20, SEQ ID NO:38, SEQ ID NO:44, SEQ ID NO:92, SEQ ID NO:96, SEQ ID NO:102, SEQ ID NO:120, SEQ ID NO:124 and SEQ ID NO:130.

18. The composition of claim 16, wherein the first nucleic acid molecule comprises a nucleotide sequence selected from the group consisting of:
a) a nucleotide sequence having at least 80% identity to a nucleotide selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and
b) a fragment of a nucleotide sequence comprising at least 80% of the full length sequence selected from the group consisting of SEQ ID NO:1, SEQ ID NO:5, SEQ ID NO:11, SEQ ID NO:17, SEQ ID NO:29, SEQ ID NO:41, SEQ ID NO:83, SEQ ID NO:93, SEQ ID NO:99, SEQ ID NO:111, SEQ ID NO:121 and SEQ ID NO:127, encoding an anti-SARS-CoV-2 antibody heavy chain; and
wherein the second nucleic acid molecule comprises a nucleotide sequence selected from the group consisting of:
c) a nucleotide sequence having at least 80% identity to a nucleotide selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain; and
d) a fragment of a nucleotide sequence comprising at least 80% of the full length sequence selected from the group consisting of SEQ ID NO:3, SEQ ID NO:7, SEQ ID NO:13, SEQ ID NO:19, SEQ ID NO:37, SEQ ID NO:43, SEQ ID NO:91, SEQ ID NO:95, SEQ ID NO:101, SEQ ID NO:119, SEQ ID NO:123 and SEQ ID NO:129, encoding an anti-SARS-CoV-2 antibody light chain.

19. The composition of claim 14, further comprising a pharmaceutically acceptable excipient.

20. The composition of any one of claim 15-18, further comprising a pharmaceutically acceptable excipient.

21. A method of preventing or treating a disease in a subject, the method comprising administering to the subject the antibody or antibody fragment of any one of claims 1-6, the nucleic acid molecule of any of claims 7-13 or a composition of any of claims 14-20.

22. The method of claim 21, wherein the disease is COVID-19.

23. The method of claim 22, further comprising administering at least one additional SARS-CoV-2 vaccine or therapeutic agent for the treatment of COVID-19 to the subject.

24. A method of inducing an immune response against SARS-CoV-2 in a subject, the method comprising administering to the subject the antibody or antibody fragment of any one of claims 1-6, the nucleic acid molecule of any of claims 7-13 or a composition of any of claims 14-20.

25. A method of inducing an immune response against SARS-CoV-2 in a subject in need thereof, the method comprising administering a combination of a first composition comprising a nucleic acid molecule encoding a synthetic anti-SARS-CoV-2 antibody, or fragment thereof of any one of claims 7-13 and a second composition comprising a nucleic acid molecule encoding a SARS-CoV-2 antigen to the subject.

26. The method of claim 25, wherein the nucleic acid molecule encoding the SARS-CoV-2 antigen comprises a nucleotide sequence encoding a peptide comprising an amino acid sequence having at least about 90% identity over an entire length of the amino acid sequence selected from the group consisting of SEQ ID NO:134, SEQ ID NO:136, SEQ ID NO:138 and SEQ ID NO:140.

27. The method of claim 26, wherein the nucleic acid molecule encoding the SARS-CoV-2 antigen comprises a nucleotide sequence encoding the amino acid sequence selected from the group consisting of SEQ ID NO:134, SEQ ID NO:136, SEQ ID NO:138 and SEQ ID NO:140.

28. The method of claim 25, wherein the nucleic acid molecule encoding the SARS-CoV-2 antigen comprises a nucleotide sequence having at least about 90% identity over an entire length of the nucleic acid sequence selected from the group consisting of SEQ ID NO:133, SEQ ID NO:135, SEQ ID NO:137 and SEQ ID NO:139.

29. The method of claim 28, wherein the nucleic acid molecule encoding the SARS-CoV-2 antigen comprises a nucleotide sequence selected from the group consisting of SEQ ID NO:133, SEQ ID NO:135, SEQ ID NO:137 and SEQ ID NO:139.

30. The method of claim 25, wherein administering includes at least one of electroporation and injection.

31. A method of treating or protecting a subject in need thereof from infection with SARS-CoV-2 or a disease or disorder associated therewith, the method comprising administering a combination of a first composition comprising a nucleic acid molecule of any one of claims 7-13 encoding a synthetic anti-SARS-CoV-2 antibody, or fragment thereof and a second composition comprising a nucleic acid molecule encoding a SARS-CoV-2 antigen to the subject.

32. The method of claim 31, wherein the disease is COVID-19.

33. The method of claim 31, wherein the nucleic acid molecule encoding the SARS-CoV-2 antigen comprises a nucleotide sequence encoding a peptide comprising an amino acid sequence having at least about 90% identity over an entire length of the amino acid sequence selected from the group consisting of SEQ ID NO:134, SEQ ID NO:136, SEQ ID NO:138 and SEQ ID NO:140.

34. The method of claim 33, wherein the nucleic acid molecule encoding the SARS-CoV-2 antigen comprises a nucleotide sequence encoding the amino acid sequence selected from the group consisting of SEQ ID NO:134, SEQ ID NO:136, SEQ ID NO:138 and SEQ ID NO:140.

35. The method of claim 31, wherein the nucleic acid molecule encoding the SARS-CoV-2 antigen comprises a nucleotide sequence having at least about 90% identity over an entire length of the nucleic acid sequence selected from the group consisting of SEQ ID NO:133, SEQ ID NO:135, SEQ ID NO:137 and SEQ ID NO:139.

36. The method of claim 35, wherein the nucleic acid molecule encoding the SARS-CoV-2 antigen comprises a nucleotide sequence selected from the group consisting of SEQ ID NO:133, SEQ ID NO:135, SEQ ID NO:137 and SEQ ID NO:139.

37. The method of claim 31, wherein administering includes at least one of electroporation and injection.


SEQUENCE LISTING (152 sequences)

SEQ ID NO: 1 - DNA, 714 bp, Artificial Sequnce
  Chemically Synethsized
atggtgctgcagactcaggtgttcatttcactgctgctgtggatttcaggagcctacggcgacattcagctgacccagagccccgattccctggccgtgtctctgggagagagggcaaccatcaactgcaagagctcccagagcgtgctgtactctagcatcaacaagaattacctggcctggtatcagcagaagcccggccagccccctaagctgctgatctattgggcaagcaccagggagtccggagtgcctgacagattctctggcagcggctccggcacagacttcaccctgacaatctcctctctgcaggccgaggacgtggccgtgtactattgccagcagtactattccaccccttacacattcggccagggcaccaaggtggagatcaagacagtggccgccccatccgtgttcatctttccaccctctgacgagcagctgaagtctggcacagccagcgtggtgtgcctgctgaacaacttctacccccgcgaggccaaggtgcagtggaaggtggataacgccctgcagtctggcaatagccaggagtccgtgaccgagcaggacaaggattctacatatagcctgagctccaccctgacactgagcaaggccgattacgagaagcacaaggtgtatgcctgtgaggtcacccatcaggggctgtcaagtccagtcacaaaatccttcaatagaggggaatgc

SEQ ID NO: 2 - protein, 238 aa, Artificial Sequnce
  Chemically Synethsized
MVLQTQVFISLLLWISGAYGDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 3 - DNA, 1398 bp, Artificial Sequnce
  Chemically Synethsized
atggactggacctggagaattctgttcctggtcgccgccgcaactgggactcacgctcagatgcagctggtgcagagtgggaccgaggtgaagaagccaggcgagtctctgaagatcagctgcaagggctccggctacggcttcatcacctattggatcggctgggtgaggcagatgcctggcaagggcctggagtggatgggcatcatctaccccggcgatagcgagacaagatattctcctagctttcagggccaggtgaccatctctgccgacaagagcatcaacacagcctacctgcagtggagctccctgaaggcctccgataccgccatctactattgcgccggcggctctggcatcagcacacctatggacgtgtggggacagggaaccacagtgaccgtggcctccacaaagggaccaagcgtgttcccactggcaccctctagcaagtccacctctggcggcacagccgccctgggctgtctggtgaaggactacttcccagagcccgtgaccgtgtcctggaactctggcgccctgacctctggcgtgcacacatttcccgccgtgctgcagtcctctggcctgtactccctgagctccgtggtgaccgtgccttctagctccctgggcacccagacatatatctgcaacgtgaatcacaagccttctaatacaaaggtggacaagaaggtggagccaaagagctgtgataagacccacacatgccctccctgtcctgcaccagagctgctgggcggcccaagcgtgttcctgtttccacccaagcccaaggataccctgatgatctcccggaccccagaggtgacatgcgtggtggtggacgtgtctcacgaggaccccgaggtgaagtttaactggtacgtggacggcgtggaggtgcacaatgccaagacaaagcccagggaggagcagtacaacagcacctatagagtggtgtccgtgctgacagtgctgcaccaggattggctgaacggcaaggagtataagtgcaaggtgagcaataaggccctgcccgcccctatcgagaagaccatctccaaggcaaagggacagcctcgggagccacaggtgtacacactgcctccaagccgcgatgagctgaccaagaaccaggtgtccctgacatgtctggtgaagggcttctatccctccgacatcgccgtggagtgggagtctaatggccagcctgagaacaattacaagaccacaccccctgtgctggacagcgatggctccttctttctgtattctaagctgaccgtggacaagagccgctggcagcagggcaacgtgttcagctgttccgtgatgcacgaagcactgcacaaccattacactcagaagtcactgtcactgtcccctggcaag

SEQ ID NO: 4 - protein, 466 aa, Artificial Sequnce
  Chemically Synethsized
MDWTWRILFLVAAATGTHAQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 384 bp, Artificial Sequnce
  Chemically Synethsized
gaggtgcagctggtgcagtcaggagccgaggtgaaggagcccggcagctccgtgaaggtgtcttgcaaggccagcggcggcaccttcggctcttacgccggcggcacatttggcagctatgccatcaactgggtgcggcaggcacctggacagcgcctggagtggatgggctggatcgatgccgccaacggcaataccaagtacagccagaagttccagggccgggtgaccatcacaggcgacacctctgccagcacagcctatatggagctgtctagcctgaggtccgaggacaccgccgtgtactattgcgcccgggatcgctggatgaccacaagagccttcgacatctggggccagggcaccatggtgacagtgtcctct

SEQ ID NO: 6 - protein, 128 aa, Artificial Sequnce
  Chemically Synethsized
EVQLVQSGAEVKEPGSSVKVSCKASGGTFGSYAGGTFGSYAINWVRQAPGQRLEWMGWIDAANGNTKYSQKFQGRVTITGDTSASTAYMELSSLRSEDTAVYYCARDRWMTTRAFDIWGQGTMVTVSS

SEQ ID NO: 7 - DNA, 333 bp, Artificial Sequnce
  Chemically Synethsized
cagcctgtgctgacacagccacccgcatccgcctctggaaccccaggccagagagtgacaatcagctgttccggcagctcctctaacatcggcagcaattacgtgttctggtatcagcagctgcccggcatggcccctaagctgctgatctcccggaacaatcagcgcccatctggcgtgcccgatcggttttctggcagcaagtccggcacctctgccagcctggcaatctccggaccacagtctgaggacgaggccgattactattgcgcagcatgggacgattccctgaggggaccagtgttcggcggcggaaccagggtgacagtgctg

SEQ ID NO: 8 - protein, 111 aa, Artificial Sequnce
  Chemically Synethsized
QPVLTQPPASASGTPGQRVTISCSGSSSNIGSNYVFWYQQLPGMAPKLLISRNNQRPSGVPDRFSGSKSGTSASLAISGPQSEDEADYYCAAWDDSLRGPVFGGGTRVTVL

SEQ ID NO: 9 - DNA, 2238 bp, Artificial Sequnce
  Chemically Synethsized
atggattggacttggaggattctgtttctggtcgccgccgctaccggaactcacgccgaggtgcagctggtgcagtcaggagccgaggtgaaggagcccggcagctccgtgaaggtgtcttgcaaggccagcggcggcaccttcggctcttacgccggcggcacatttggcagctatgccatcaactgggtgcggcaggcacctggacagcgcctggagtggatgggctggatcgatgccgccaacggcaataccaagtacagccagaagttccagggccgggtgaccatcacaggcgacacctctgccagcacagcctatatggagctgtctagcctgaggtccgaggacaccgccgtgtactattgcgcccgggatcgctggatgaccacaagagccttcgacatctggggccagggcaccatggtgacagtgtcctctgcctctacaaagggaccaagcgtgtttccactggcacccagctccaagtccacctctggcggcacagccgccctgggctgtctggtgaaggattacttccccgagcctgtgaccgtgtcctggaactctggcgccctgaccagcggagtgcacacatttcctgccgtgctgcagtctagcggcctgtactccctgtcctctgtggtgaccgtgccaagctcctctctgggcacccagacatatatctgcaacgtgaatcacaagccctctaatacaaaggtggataagaaggtggagcctaagagctgtgacaagacccacacatgccctccctgtccagcaccagagctgctgggcggccctagcgtgttcctgtttccacccaagccaaaggataccctgatgatctcccgcaccccagaggtgacatgcgtggtggtggacgtgtctcacgaggaccccgaggtgaagttcaactggtacgtggacggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcctgccccaatcgagaagaccatctccaaggcaaagggacagcctcgggagccacaggtgtacacactgcctccaagccgcgatgagctgaccaagaaccaggtgtccctgacatgtctggtgaagggcttctatccatccgacatcgccgtggagtgggagtctaatggccagcccgagaacaattacaagaccacaccccctgtgctggacagcgatggctccttctttctgtattctaagctgaccgtggataagagcaggtggcagcagggcaacgtgtttagctgctccgtgatgcacgaggccctgcacaatcactacacccagaagtctctgagcctgtccccaggcaagaggggcagaaagaggagatctggcagcggcgccacaaacttcagcctgctgaagcaggcaggcgacgtggaggagaatccaggacctatggtgctgcagacccaggtgtttatcagcctgctgctgtggatctccggagcatacggacagcctgtgctgacacagccacccgcatccgcctctggaaccccaggccagagagtgacaatcagctgttccggcagctcctctaacatcggcagcaattacgtgttctggtatcagcagctgcccggcatggcccctaagctgctgatctcccggaacaatcagcgcccatctggcgtgcccgatcggttttctggcagcaagtccggcacctctgccagcctggcaatctccggaccacagtctgaggacgaggccgattactattgcgcagcatgggacgattccctgaggggaccagtgttcggcggcggaaccagggtgacagtgctgagaaccgtggcagcaccaagcgtgttcatctttcctccatccgatgagcagctgaagagcggcacagcctccgtggtgtgcctgctgaacaacttctaccccagggaggccaaggtgcagtggaaggtggacaacgccctgcagtctggcaatagccaggagtccgtgaccgagcaggactctaaggatagcacatattccctgagctccaccctgacactgtccaaggccgactacgagaagcacaaggtgtatgcctgcgaagtcacccaccaggggctgaggtctccagtcactaaatctttcaataggggcgaatgctgataa

SEQ ID NO: 10 - protein, 744 aa, Artificial Sequnce
  Chemically Synethsized
MDWTWRILFLVAAATGTHAEVQLVQSGAEVKEPGSSVKVSCKASGGTFGSYAGGTFGSYAINWVRQAPGQRLEWMGWIDAANGNTKYSQKFQGRVTITGDTSASTAYMELSSLRSEDTAVYYCARDRWMTTRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGQPVLTQPPASASGTPGQRVTISCSGSSSNIGSNYVFWYQQLPGMAPKLLISRNNQRPSGVPDRFSGSKSGTSASLAISGPQSEDEADYYCAAWDDSLRGPVFGGGTRVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 11 - DNA, 372 bp, Artificial Sequnce
  Chemically Synethsized
caggtgcagctggtgcagtctggggccgaagtgaagaagccaggcagctccgtgaaggtgtcctgcaaggcctctggcggcacattctctagctacgccatctcctgggtgaggcaggcaccaggacagggcctggagtgggtgggcagaatcatccctatcttcggcaccgccaactacgcccagtttcagggccgggtgaccatcacagccgacaagtccacctctacagcctatatggagctgtcctctctgaggagcgaggataccgccgtgtactattgcgccagagccagctactgctccaccacatcttgtgcaagcggagccttcgacatctggggacagggcaccctggtgacagtgagctcc

SEQ ID NO: 12 - protein, 124 aa, Artificial Sequnce
  Chemically Synethsized
QVQLVQSGAEVKKPGSSVKVSCKASGGTFSSYAISWVRQAPGQGLEWVGRIIPIFGTANYAQFQGRVTITADKSTSTAYMELSSLRSEDTAVYYCARASYCSTTSCASGAFDIWGQGTLVTVSS

SEQ ID NO: 13 - DNA, 339 bp, Artificial Sequnce
  Chemically Synethsized
gacatccagatgacccagtctcctgatagcctggccgtgtccctgggagagagagcaacaatcaactgtaagtctagccagagcgtgctgtactcctctaacaataagaattacctggcctggtatcagcagaagccaggccagccacccaagctgctgatctattgggcaagcgccagggagtccggagtgccagacagattcagcggctccggctctggaaccgacttcaccctgacaatcagctccctgcagcccgaggacgtggccatctactattgccagcagtactattccgtgcccttcacctttggccctggcacaaaggtggagatcaag

SEQ ID NO: 14 - protein, 113 aa, Artificial Sequnce
  Chemically Synethsized
DIQMTQSPDSLAVSLGERATINCKSSQSVLYSSNNKNYLAWYQQKPGQPPKLLIYWASARESGVPDRFSGSGSGTDFTLTISSLQPEDVAIYYCQQYYSVPFTFGPGTKVEIK

SEQ ID NO: 15 - DNA, 2232 bp, Artificial Sequnce
  Chemically Synethsized
atggattggacatggaggattctgtttctggtcgccgccgccacaggaactcacgctcaggtgcagctggtgcagtctggggccgaagtgaagaagccaggcagctccgtgaaggtgtcctgcaaggcctctggcggcacattctctagctacgccatctcctgggtgaggcaggcaccaggacagggcctggagtgggtgggcagaatcatccctatcttcggcaccgccaactacgcccagtttcagggccgggtgaccatcacagccgacaagtccacctctacagcctatatggagctgtcctctctgaggagcgaggataccgccgtgtactattgcgccagagccagctactgctccaccacatcttgtgcaagcggagccttcgacatctggggacagggcaccctggtgacagtgagctccgcctccacaaagggaccaagcgtgttcccactggcaccctctagcaagagcacctccggcggcacagccgccctgggctgtctggtgaaggattatttccccgagcctgtgaccgtgtcctggaactctggcgccctgacctccggagtgcacacatttcccgccgtgctgcagtcctctggcctgtacagcctgagctccgtggtgaccgtgccttctagctccctgggcacccagacatatatctgcaacgtgaatcacaagcctagcaatacaaaggtggacaagaaggtggagccaaagtcctgtgataagacccacacatgccctccctgtccagcaccagagctgctgggcggcccaagcgtgttcctgtttccacccaagcccaaggacacactgatgatctctcgcacccctgaggtgacatgcgtggtggtggacgtgagccacgaggaccccgaggtgaagtttaactggtacgtggatggcgtggaggtgcacaatgccaagaccaagcctcgggaggagcagtacaacagcacctatcgcgtggtgtccgtgctgacagtgctgcaccaggactggctgaacggcaaggagtataagtgcaaggtgtccaataaggccctgcctgccccaatcgagaagaccatctctaaggcaaagggacagcctcgggagccacaggtgtacacactgcctccatcccgcgacgagctgaccaagaaccaggtgtctctgacatgtctggtgaagggcttctatccatctgatatcgccgtggagtgggagagcaatggccagcccgagaacaattacaagaccacaccccctgtgctggacagcgatggctccttctttctgtattccaagctgaccgtggacaagtctaggtggcagcagggcaacgtgttttcttgcagcgtgatgcacgaggccctgcacaatcactacacccagaagtccctgtctctgagcccaggcaagaggggaaggaagaggagatccggctctggcgccacaaacttcagcctgctgaagcaggcaggcgatgtggaggagaatccaggacctatggtgctgcagacccaggtgtttatctctctgctgctgtggatcagcggcgcctacggcgacatccagatgacccagtctcctgatagcctggccgtgtccctgggagagagagcaacaatcaactgtaagtctagccagagcgtgctgtactcctctaacaataagaattacctggcctggtatcagcagaagccaggccagccacccaagctgctgatctattgggcaagcgccagggagtccggagtgccagacagattcagcggctccggctctggaaccgacttcaccctgacaatcagctccctgcagcccgaggacgtggccatctactattgccagcagtactattccgtgcccttcacctttggccctggcacaaaggtggagatcaagaggaccgtggcagcacctagcgtgttcatctttcctccatccgacgagcagctgaagtctggcacagccagcgtggtgtgcctgctgaacaatttctacccacgcgaggccaaggtgcagtggaaggtggataacgccctgcagtccggcaattctcaggagagcgtgaccgagcaggactccaaggattctacatatagcctgtctagcaccctgacactgagcaaggccgattacgagaagcacaaggtgtatgcctgcgaagtcacccaccaggggctgaggtcaccagtcaccaagtctttcaacagaggggaatgttgataa

SEQ ID NO: 16 - protein, 742 aa, Artificial Sequnce
  Chemically Synethsized
MDWTWRILFLVAAATGTHAQVQLVQSGAEVKKPGSSVKVSCKASGGTFSSYAISWVRQAPGQGLEWVGRIIPIFGTANYAQFQGRVTITADKSTSTAYMELSSLRSEDTAVYYCARASYCSTTSCASGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPDSLAVSLGERATINCKSSQSVLYSSNNKNYLAWYQQKPGQPPKLLIYWASARESGVPDRFSGSGSGTDFTLTISSLQPEDVAIYYCQQYYSVPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 17 - DNA, 375 bp, Artificial Sequnce
  Chemically Synethsized
caggtgcagctggtgcagtctggggctgaagtgaagaaaccaggcagctccgtcaaggtgtcatgcaaagccagcggcggaacattctctagttacgcaatcagctgggtgcgacaggctccaggacagggactggagtggatgggcggcatcattcctatcttcgggaccgctaactacgcacagaagtttcagggccgcgtgaccattacagcagataaatccacttctaccgcctatatggagctgtcaagcctgaggagcgaagacactgcagtctactattgcgccagagtgggatactgctcctctacctcctgtcacatcggggccttcgatatttggggacaggggaccacagtcaccgtgagttca

SEQ ID NO: 18 - protein, 125 aa, Artificial Sequnce
  Chemically Synethsized
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARVGYCSSTSCHIGAFDIWGQGTTVTVSS

SEQ ID NO: 19 - DNA, 321 bp, Artificial Sequnce
  Chemically Synethsized
gaaacaactctgactcagagccccggcaccctgtctctgagtcctggagagcgagctaccctgagctgtagggcatcacagagcgtgtcaagctccatcgcctggtatcagcagaagcctggacaggctccacgcctgctgatgttcgactctagtacaagagctactggcatccccgatcggttctccgggtctggcagtggaaccgactttacactgaacatttcaagcctggagcccgaagatttcgcagtgtactattgccagcagtactcctctagtccttatacatttgggcagggcactaagctggaaatcaaa

SEQ ID NO: 20 - protein, 107 aa, Artificial Sequnce
  Chemically Synethsized
ETTLTQSPGTLSLSPGERATLSCRASQSVSSSIAWYQQKPGQAPRLLMFDSSTRATGIPDRFSGSGSGTDFTLNISSLEPEDFAVYYCQQYSSSPYTFGQGTKLEIK

SEQ ID NO: 21 - DNA, 2216 bp, Artificial Sequnce
  Chemically Synethsized
atggattggacatggagaattctgtttctggtcgccgccgccactggaactcacgctcaggtgcagctggtgcagtctggggctgaagtgaagaaaccaggcagctccgtcaaggtgtcatgcaaagccagcggcggaacattctctagttacgcaatcagctgggtgcgacaggctccaggacagggactggagtggatgggcggcatcattcctatcttcgggaccgctaactacgcacagaagtttcagggccgcgtgaccattacagcagataaatccacttctaccgcctatatggagctgtcaagcctgaggagcgaagacactgcagtctactattgcgccagagtgggatactgctcctctacctcctgtcacatcggggccttcgatatttggggacaggggaccacagtcaccgtgagttcagcaagtacaaaggggccttcagtgtttcccctggcccctagctccaaaagtacttcaggagggaccgccgctctgggatgtctggtgaaggactatttccctgagccagtcaccgtgtcatggaacagcggggccctgacctccggagtccatacatttccagctgtgctgcagtctagtgggctgtactctctgtcaagcgtggtcactgtcccctcctctagtctgggcacacagacttatatctgcaacgtgaatcacaagccttccaataccaaagtcgacaagaaagtggaaccaaagtcttgtgataaaacccatacatgccctccctgtccagcacctgagctgctgggcggaccaagcgtgttcctgtttccacccaagcccaaagatacactgatgattagccggacacctgaagtcacttgcgtggtcgtggacgtgagccacgaggaccccgaagtcaagttcaactggtacgtggacggcgtcgaggtgcataatgctaagaccaaaccccgcgaggaacagtacaatagcacatatcgagtcgtgtccgtcctgactgtgctgcaccaggactggctgaacggaaaggagtataagtgcaaagtgtccaataaggccctgccagctcccatcgagaaaacaatttctaaggccaaaggccagccacgggaaccccaggtgtacactctgcctccaagccgcgatgagctgactaagaaccaggtcagcctgacctgtctggtgaaaggcttctatccaagtgacatcgctgtggagtgggaatctaatggacagcccgaaaacaattacaagactacccctcccgtgctggactccgatggctctttctttctgtattcaaagctgacagtggataaaagccggtggcagcagggaaacgtctttagctgctccgtgatgcatgaggccctgcacaatcattacacacagaagtctctgagtctgtcacccggaaagcgaggacgaaaaaggagaagcggctccggagctactaacttctccctgctgaagcaggcaggagacgtggaggaaaatcctgggccaatggtcctgcagacccaggtgtttatctctctgctgctgtggattagtggcgcttacggagaaacaactctgactcagagccccggcaccctgtctctgagtcctggagagcgagctaccctgagctgtagggcatcacagagcgtgtcaagctccatcgcctggtatcagcagaagcctggacaggctccacgcctgctgatgttcgactctagtacaagagctactggcatccccgatcggttctccgggtctggcagtggaaccgactttacactgaacatttcaagcctggagcccgaagatttcgcagtgtactattgccagcagtactcctctagtccttatacatttgggcagggcactaagctggaaatcaaaaggaccgtcgcagccccttccgtgttcatttttccaccctctgatgagcagctgaagtccggcacagcctctgtggtgtgcctgctgaacaacttctacccaagagaagcaaaggtccagtggaaagtggacaacgccctgcagagtggcaattcacaggagagcgtgaccgaacaggactccaaggattctacatatagtctgtcaagcactctgaccctagcaaagctgactacgagaagcacaaagtgtatgcatgcgaagtgacccaccaggggctgagaagtccagtgacaaaatcctttaacagaggggaatgctgataa

SEQ ID NO: 22 - protein, 737 aa, Artificial Sequnce
  Chemically Synethsized
MDWTWRILFLVAAATGTHAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARVGYCSSTSCHIGAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCRASQSVSSSIAWYQQKPGQAPRLLMFDSSTRATGIPDRFSGSGSGTDFTLNISSLEPEDFAVYYCQQYSSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 23 - DNA, 15 bp, Artificial Sequnce
  Chemically Synethsized
tcctacggaatgcac

SEQ ID NO: 24 - DNA, 48 bp, Artificial Sequnce
  Chemically Synethsized
atctcttatgacggcagcaacaagtactatgccgatagcgtgaagggc

SEQ ID NO: 25 - DNA, 60 bp, Artificial Sequnce
  Chemically Synethsized
gacggatatggcagcggctccgattactactactactactacatggacgtgtggggcaag

SEQ ID NO: 26 - protein, 5 aa, Artificial Sequnce
  Chemically Synethsized
SYGMH

SEQ ID NO: 27 - protein, 16 aa, Artificial Sequnce
  Chemically Synethsized
ISYDGSNKYYADSVKG

SEQ ID NO: 28 - protein, 20 aa, Artificial Sequnce
  Chemically Synethsized
DGYGSGSDYYYYYYMDVWGK

SEQ ID NO: 29 - DNA, 399 bp, Artificial Sequnce
  Chemically Synethsized
gaggtgcagctggtcgagactggagggggcgtggtgcagccaggccggagcctgagactgagctgcgcagcatccggcttcacctttagctcctacggaatgcactgggtgaggcaggcaccaggcaagggcctggagtgggtggccgtgatctcttatgacggcagcaacaagtactatgccgatagcgtgaagggcaggttcaccatctcccgcgacaactctaagaatacactgtacctgcagatgaattccctgagggccgaggataccgccgtgtactattgcgcccgcgacggatatggcagcggctccgattactactactactactacatggacgtgtggggcaagggcaccatggtgacagtgtctagctccgccagcggagcagagctg

SEQ ID NO: 30 - protein, 133 aa, Artificial Sequnce
  Chemically Synethsized
EVQLVETGGGVVQPGRSLRLSCAASGFTFSSYGMHWVRQAPGKGLEWVAVISYDGSNKYYADSVKGRFTISRDNSKNTLYLQMNSLRAEDTAVYYCARDGYGSGSDYYYYYYMDVWGKGTMVTVSSSASGAEL

SEQ ID NO: 31 - DNA, 30 bp, Artificial Sequnce
  Chemically Synethsized
cagggcgatagcctgagatcctactatgcc

SEQ ID NO: 32 - DNA, 21 bp, Artificial Sequnce
  Chemically Synethsized
ggcaagaacaatcggccctcc

SEQ ID NO: 33 - DNA, 30 bp, Artificial Sequnce
  Chemically Synethsized
aactccagggattctagcggcaatcaccct

SEQ ID NO: 34 - protein, 10 aa, Artificial Sequnce
  Chemically Synethsized
QGDSLRSYYA

SEQ ID NO: 35 - protein, 7 aa, Artificial Sequnce
  Chemically Synethsized
GKNNRPS

SEQ ID NO: 36 - protein, 10 aa, Artificial Sequnce
  Chemically Synethsized
NSRDSSGNHP

SEQ ID NO: 37 - DNA, 324 bp, Artificial Sequnce
  Chemically Synethsized
acctctagctccgagctgacacaggaccctgccgtgagcgtggccctgggacagaccgtgcggatcacatgtcagggcgatagcctgagatcctactatgcctcctggtaccagcagaagccaggacaggcaccagtgcctgtgatctatggcaagaacaatcggccctccggcatccctgacagattctctggctctagctccggcaacaccgcaagcctgaccatcacaggagcacaggcagaggacgaggcagattactattgcaactccagggattctagcggcaatcacccttttggcaccggcacaaaggtgaccctg

SEQ ID NO: 38 - protein, 108 aa, Artificial Sequnce
  Chemically Synethsized
TSSSELTQDPAVSVALGQTVRITCQGDSLRSYYASWYQQKPGQAPVPVIYGKNNRPSGIPDRFSGSSSGNTASLTITGAQAEDEADYYCNSRDSSGNHPFGTGTKVTL

SEQ ID NO: 39 - DNA, 2244 bp, Artificial Sequnce
  Chemically Synethsized
atggactggacttggaggattctgtttctggtcgccgccgctactgggactcacgccgaggtgcagctggtcgagactggagggggcgtggtgcagccaggccggagcctgagactgagctgcgcagcatccggcttcacctttagctcctacggaatgcactgggtgaggcaggcaccaggcaagggcctggagtgggtggccgtgatctcttatgacggcagcaacaagtactatgccgatagcgtgaagggcaggttcaccatctcccgcgacaactctaagaatacactgtacctgcagatgaattccctgagggccgaggataccgccgtgtactattgcgcccgcgacggatatggcagcggctccgattactactactactactacatggacgtgtggggcaagggcaccatggtgacagtgtctagctccgccagcggagcagagctggcatctacaaagggaccaagcgtgtttccactggcaccctctagcaagtctaccagcggcggcacagccgccctgggatgtctggtgaaggattacttccctgagccagtgaccgtgtcttggaacagcggcgccctgaccagcggagtgcacacatttcccgccgtgctgcagtcctctggcctgtactccctgagctccgtggtgaccgtgccttctagctccctgggcacccagacatatatctgcaacgtgaatcacaagcctagcaatacaaaggtggacaagaaggtggagccaaagtcctgtgataagacccacacatgccctccctgtccagcacctgagctgctgggcggcccatccgtgttcctgtttccacccaagcccaaggacacactgatgatctctcggaccccagaggtgacatgcgtggtggtggacgtgagccacgaggaccccgaggtgaagttcaactggtacgtggatggcgtggaggtgcacaatgccaagaccaagcccagggaggagcagtacaactccacctatcgcgtggtgtctgtgctgacagtgctgcaccaggactggctgaacggcaaggagtataagtgcaaggtgtccaataaggccctgccagcccccatcgagaagaccatctctaaggcaaagggacagcctcgggagccacaggtgtacacactgcctccatccagagacgagctgaccaagaaccaggtgtctctgacatgtctggtgaagggcttctatccctctgatatcgccgtggagtgggagagcaatggccagcctgagaacaattacaagaccacaccccctgtgctggactccgatggctctttctttctgtattctaagctgaccgtggacaagagcagatggcagcagggcaacgtgttttcctgctctgtgatgcacgaggccctgcacaatcactacacccagaagagcctgtccctgtctcctggcaagaggggaaggaagcggagaagcggctccggagccacaaacttcagcctgctgaagcaggccggcgatgtggaggagaatcctggcccaatggtgctgcagacccaggtgtttatcagcctgctgctgtggatctccggagcatacggcacctctagctccgagctgacacaggaccctgccgtgagcgtggccctgggacagaccgtgcggatcacatgtcagggcgatagcctgagatcctactatgcctcctggtaccagcagaagccaggacaggcaccagtgcctgtgatctatggcaagaacaatcggccctccggcatccctgacagattctctggctctagctccggcaacaccgcaagcctgaccatcacaggagcacaggcagaggacgaggcagattactattgcaactccagggattctagcggcaatcacccttttggcaccggcacaaaggtgaccctgaggacagtggcagcaccaagcgtgttcatctttccaccctccgacgagcagctgaagagcggaaccgcatccgtggtgtgcctgctgaacaacttctacccccgggaggccaaggtgcagtggaaggtggataacgccctgcagtctggcaatagccaggagtccgtgaccgagcaggactctaaggatagcacatattccctgtcctctaccctgacactgagcaaggccgactacgagaagcacaaggtgtatgcctgcgaagtcacccaccaggggctgcggtcaccagtcacaaaatcctttaatagaggcgaatgttgataa

SEQ ID NO: 40 - protein, 746 aa, Artificial Sequnce
  Chemically Synethsized
MDWTWRILFLVAAATGTHAEVQLVETGGGVVQPGRSLRLSCAASGFTFSSYGMHWVRQAPGKGLEWVAVISYDGSNKYYADSVKGRFTISRDNSKNTLYLQMNSLRAEDTAVYYCARDGYGSGSDYYYYYYMDVWGKGTMVTVSSSASGAELASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGTSSSELTQDPAVSVALGQTVRITCQGDSLRSYYASWYQQKPGQAPVPVIYGKNNRPSGIPDRFSGSSSGNTASLTITGAQAEDEADYYCNSRDSSGNHPFGTGTKVTLRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 41 - DNA, 1425 bp, Artificial Sequnce
  Chemically Synethsized
atggactggacctggagaatcctgttcctggtggcagcagcaaccggaacacacgcacaggtgcagctggtgcagtccggagcagaggtgaagaagcctggagcctctgtgaaggtgagctgcaaggcctccggctacacctttacatcttatggaatcagctgggtgaggcaggcaccaggacagggactggagtggatgggctggatcagcgcctacaacggcaatacaaactatgcccagaagctgcagggcagagtgaccatgaccacagacaccagcacatccaccgcctacatggagctgaggtctctgagaagcgacgatacagccgtgtactattgcgcccgggactatacccgcggcgcctggttcggagagtctctgatcggcggatttgataattggggccagggcacactggtgaccgtgtctgccagcacaaagggaccaagcgtgttcccactggcacccagctccaagtccacatctggcggcaccgccgccctgggatgtctggtgaaggattacttcccagagcccgtgaccgtgtcctggaattctggcgccctgacaagcggcgtgcacacctttccagccgtgctgcagtctagcggcctgtactccctgtcctctgtggtgacagtgcccagctcctctctgggcacacagacctatatctgcaatgtgaaccacaagccaagcaacaccaaggtggacaagaaggtggagcccaagtcctgtgataagacacacacctgccctccctgtcctgcaccagagctgctgggcggcccatccgtgttcctgtttccacccaagcctaaggacaccctgatgatctctcggacacccgaggtgacctgcgtggtggtggacgtgagccacgaggaccccgaggtgaagtttaattggtacgtggatggcgtggaggtgcacaacgccaagaccaagcccagggaggagcagtacaactccacatatagagtggtgtctgtgctgaccgtgctgcaccaggactggctgaatggcaaggagtataagtgcaaggtgtccaacaaggccctgcccgcccctatcgagaagacaatctctaaggcaaagggacagcctcgggagccacaggtgtacaccctgcctccatcccgcgacgagctgacaaagaatcaggtgtctctgacctgtctggtgaagggcttctatccttctgatatcgcagtggagtgggagagcaacggacagccagagaacaattacaagaccacaccccctgtgctggacagcgatggctccttctttctgtatagcaagctgacagtggataagtcccgctggcagcagggcaacgtgttcagctgttccgtgatgcacgaggccctgcacaaccactacacccagaagtctctgagcctgtcccctggcaag

SEQ ID NO: 42 - protein, 475 aa, Artificial Sequnce
  Chemically Synethsized
MDWTWRILFLVAAATGTHAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYTRGAWFGESLIGGFDNWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 699 bp, Artificial Sequnce
  Chemically Synethsized
atggtgctgcagacccaggtgttcatctctctgctgctgtggatcagcggagcatacggagagatcgtgctgacccagagccctggcacactgagcctgtccccaggagagagggccacactgtcctgcagagcctctcagagcgtgagctcctcttacctggcctggtatcagcagaagccaggacaggcccccaggctgctgatctatggagccagctcccgggccaccggcatccccgaccgcttctccggctctggcagcggcacagacttcaccctgacaatctctaggctggagcctgaggacttcgccgtgtactattgccagcagcacgataccagcctgacatttggccagggcaccaaggtggagatcaagagaacagtggccgccccatccgtgttcatctttcccccttctgacgagcagctgaagagcggaaccgcatccgtggtgtgcctgctgaacaatttctacccccgggaggccaaggtgcagtggaaggtggataacgccctgcagtccggcaattctcaggagagcgtgaccgagcaggacaaggattccacatattctctgtctagcaccctgacactgtccaaggccgactacgagaagcacaaggtgtatgcatgcgaggtgacccaccagggactgtcctctcccgtgacaaagagctttaaccgcggcgagtgt

SEQ ID NO: 44 - protein, 233 aa, Artificial Sequnce
  Chemically Synethsized
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQQHDTSLTFGQGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 45 - DNA, 1425 bp, Artificial Sequnce; identical to SEQ ID NO: 41
  Chemically Synethsized

SEQ ID NO: 46 - protein, 748 aa, Artificial Sequnce
  Chemically Synethsized
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 47 - DNA, 2244 bp, Artificial Sequnce
  Chemically Synethsized
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacggcgccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcaacaccctgacactgagcaaggccgactatgagaagcacaaggtgtacgcctgcgaagtgacccaccaggggctgagcagtccagtgaccaagtctttcaatcggggagaatgc

SEQ ID NO: 48 - protein, 748 aa, Artificial Sequnce
  Chemically Synethsized
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 49 - DNA, 2244 bp, Artificial Sequnce
  Chemically Synethsized
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccaggaagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacggcgccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcaacaccctgacactgagcaaggccgactatgagaagcacaaggtgtacgcctgcgaagtgacccaccaggggctgagcagtccagtgaccaagtctttcaatcggggagaatgc

SEQ ID NO: 50 - protein, 748 aa, Artificial Sequnce
  Chemically Synethsized
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 51 - DNA, 2244 bp, Artificial Sequnce
  Chemically Synethsized
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagttcgagggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccagcatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcaacaccctgacactgagcaaggccgactatgagaagcacaaggtgtacgcctgcgaagtgacccaccaggggctgagcagtccagtgaccaagtctttcaatcggggagaatgc

SEQ ID NO: 52 - protein, 748 aa, Artificial Sequnce
  Chemically Synethsized
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 53 - DNA, 2211 bp, Artificial Sequnce
  Chemically Synethsized
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacgaggccctgcacaatcactatacccagaagtctctgagcctgtcccctggcaagaggggaaggaagaggagatctggcagcggcgccacaaactttagcctgctgaagcaggccggcgatgtggaggagaatcctggcccaatggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgt

SEQ ID NO: 54 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 55 - DNA, 2211 bp, Artificial Sequnce
  Chemically Synethsized
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacggcgccctgcacaatcactatacccagaagtctctgagcctgtcccctggcaagaggggaaggaagaggagatctggcagcggcgccacaaactttagcctgctgaagcaggccggcgatgtggaggagaatcctggcccaatggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgt

SEQ ID NO: 56 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 57 - DNA, 2211 bp, Artificial Sequnce
  Chemically Synethsized
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcaagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacggcgccctgcacaatcactatacccagaagtctctgagcctgtcccctggcaagaggggaaggaagaggagatctggcagcggcgccacaaactttagcctgctgaagcaggccggcgatgtggaggagaatcctggcccaatggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgt

SEQ ID NO: 58 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 59 - DNA, 2211 bp, Artificial Sequnce
  Chemically Synethsized
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagttcgagggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacgaggccctgcacaatcactatacccagaagtctctgagcctgtcccctggcaagaggggaaggaagaggagatctggcagcggcgccacaaactttagcctgctgaagcaggccggcgatgtggaggagaatcctggcccaatggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgt

SEQ ID NO: 60 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 61 - DNA, 2211 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagttccaggagcgcgtgtctatcacaagggacatgtccacctctacagcctatatggagctgtctagcctgcggtccgaggatacagccgtgtactattgcgccgccccttactgttcctctatcagctgcaacgacggcttcgatatctggggccagggcaccaaggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagaggtggtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtaccactgtcagcactatggctcctctagccagtggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgt

SEQ ID NO: 62 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSSISCNDGFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHYGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 63 - DNA, 2211 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagttccaggagcgcgtgtctatcacaagggacatgtccacctctacagcctatatggagctgtctagcctgcggtccgaggatacagccgtgtactattgcgccgccccttactgttcctctatcagctgcaacgacggcttcgatatctggggccagggcaccaaggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacggcgccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagaggtggtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtaccactgtcagcactatggctcctctagccagtggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgt

SEQ ID NO: 64 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSSISCNDGFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHYGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 65 - DNA, 2211 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagttccaggagcgcgtgtctatcacaagggacatgtccacctctacagcctatatggagctgtctagcctgcggtccgaggatacagccgtgtactattgcgccgccccttactgttcctctatcagctgcaacgacggcttcgatatctggggccagggcaccaaggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcaagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacggcgccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagaggtggtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtaccactgtcagcactatggctcctctagccagtggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgt

SEQ ID NO: 66 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSSISCNDGFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHYGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 67 - DNA, 2211 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagttccaggagcgcgtgtctatcacaagggacatgtccacctctacagcctatatggagctgtctagcctgcggtccgaggatacagccgtgtactattgcgccgccccttactgttcctctatcagctgcaacgacggcttcgatatctggggccagggcaccaaggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagttcgagggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagaggtggtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtaccactgtcagcactatggctcctctagccagtggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgt

SEQ ID NO: 68 - protein, 737 aa, Artificial Sequnce
  Chemically Synethsized
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSSISCNDGFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHYGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 69 - DNA, 2217 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 70 - protein, 737 aa, Artificial Sequnce
  Chemically Synethsized
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 71 - DNA, 2217 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacggcgccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 72 - protein, 737 aa, Artificial Sequnce
  Chemically Synethsized
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 73 - DNA, 2217 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcaagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacggcgccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 74 - protein, 737 aa, Artificial Sequnce
  Chemically Synethsized
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 75 - DNA, 2217 bp, Artificial Sequnce
  Chemically Synethsized
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagttcgagggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 76 - protein, 737 aa, Artificial Sequnce; identical to SEQ ID NO: 68
  Chemically Synethsized

SEQ ID NO: 77 - DNA, 15 bp, Artificial Sequnce
  Chemically Synethsized
agatactggatgagt

SEQ ID NO: 78 - DNA, 51 bp, Artificial Sequnce
  Chemically Synethsized
gaaattaatccagatagcagtacgataaactatacgccatctctaaaggat

SEQ ID NO: 79 - DNA, 33 bp, Artificial Sequnce
  Chemically Synethsized
atctactatggttcccctggtgctatggactac

SEQ ID NO: 80 - protein, 5 aa, Artificial Sequnce
  Chemically Synethsized
RYWMS

SEQ ID NO: 81 - protein, 17 aa, Artificial Sequnce
  Chemically Synethsized
EINPDSSTINYTPSLKD

SEQ ID NO: 82 - protein, 11 aa, Artificial Sequnce
  Chemically Synethsized
IYYGSPGAMDY

SEQ ID NO: 83 - DNA, 414 bp, Artificial Sequnce
  Chemically Synethsized
atggattttgggctgattttttttattgttgctcttttaaaaggggtccagtgtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaaggatctactatggttcccctggtgctatggactactggggtcaaggaacctcagtcaccgtctcctca

SEQ ID NO: 84 - protein, 138 aa, Artificial Sequnce
  Chemically Synethsized
MDFGLIFFIVALLKGVQCEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARIYYGSPGAMDYWGQGTSVTVSS

SEQ ID NO: 85 - DNA, 33 bp, Artificial Sequnce
  Chemically Synethsized
aaggccagtcagagtgtgagtaatgatgtagct

SEQ ID NO: 86 - DNA, 21 bp, Artificial Sequnce
  Chemically Synethsized
tatgcatccaatcgctacact

SEQ ID NO: 87 - DNA, 27 bp, Artificial Sequnce
  Chemically Synethsized
cagcaggattatttctctccgtacacg

SEQ ID NO: 88 - protein, 11 aa, Artificial Sequnce
  Chemically Synethsized
KASQSVSNDVA

SEQ ID NO: 89 - protein, 7 aa, Artificial Sequnce
  Chemically Synethsized
YASNRYT

SEQ ID NO: 90 - protein, 9 aa, Artificial Sequnce
  Chemically Synethsized
QQDYFSPYT

SEQ ID NO: 91 - DNA, 381 bp, Artificial Sequnce
  Chemically Synethsized
atgaagtcacagacccaggtcttcgtatttctactgctctgtgtgtctggtgctcatgggagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatttctctccgtacacgttcggaggggggaccaagctggaaataaaa

SEQ ID NO: 92 - protein, 127 aa, Artificial Sequnce
  Chemically Synethsized
MKSQTQVFVFLLLCVSGAHGSIVMTQTPKFLLVSAGDRVTITCKASQSVSNDVAWYQQKPGQSPKLLIYYASNRYTGVPDRFTGSGYGTDFTFTISTVQAEDLAVYFCQQDYFSPYTFGGGTKLEIK

SEQ ID NO: 93 - DNA, 360 bp, Artificial Sequnce
  Chemically Synethsized
gaggtgaagctgctggagagcggaggaggactggtgcagcctggaggcagcctgaagctgtcctgcgctgcctctggattcgacttttccaggtactggatgtcttgggtgagacaggccccaggcaagggactggagtggatcggcgagatcaacccagatagctccacaatcaactacaccccctccctgaaggacaagttcatcatctctagagataacgctaagaacacactgtacctgcagatgagcaaggtgaggtccgaggacaccgctctgtactactgcgccagaatctactacggcagccctggcgccatggattactggggccagggaacatctgtgaccgtgagcagc

SEQ ID NO: 94 - protein, 120 aa, Artificial Sequnce
  Chemically Synethsized
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARIYYGSPGAMDYWGQGTSVTVSS

SEQ ID NO: 95 - DNA, 321 bp, Artificial Sequnce
  Chemically Synethsized
tccatcgtgatgacacagacccccaagtttctgctggtgtccgccggcgacagggtgacaatcacctgtaaggcttcccagtctgtgagcaacgatgtggcctggtaccagcagaagcctggacagtccccaaagctgctgatctactacgcctctaaccggtacacaggcgtgcctgaccgcttcacaggctctggatacggcaccgacttcaccttcaccatcagcaccgtgcaggctgaggacctggccgtgtacttttgccagcaggattacttcagcccatacacatttggaggcggaaccaagctggagatcaag

SEQ ID NO: 96 - protein, 107 aa, Artificial Sequnce
  Chemically Synethsized
SIVMTQTPKFLLVSAGDRVTITCKASQSVSNDVAWYQQKPGQSPKLLIYYASNRYTGVPDRFTGSGYGTDFTFTISTVQAEDLAVYFCQQDYFSPYTFGGGTKLEIK

SEQ ID NO: 97 - DNA, 2178 bp, Artificial Sequnce
  Chemically Synethsized
atggactggacctggagaatcctgttcctggtggctgctgctaccggaacacacgctgaggtgaagctgctggagagcggaggaggactggtgcagcctggaggcagcctgaagctgtcctgcgctgcctctggattcgacttttccaggtactggatgtcttgggtgagacaggccccaggcaagggactggagtggatcggcgagatcaacccagatagctccacaatcaactacaccccctccctgaaggacaagttcatcatctctagagataacgctaagaacacactgtacctgcagatgagcaaggtgaggtccgaggacaccgctctgtactactgcgccagaatctactacggcagccctggcgccatggattactggggccagggaacatctgtgaccgtgagcagcgccaagaccacaccacctagcgtgtacccactggctcctggctccgctgctcagacaaactctatggtgaccctgggatgtctggtgaagggctacttccctgagccagtgaccgtgacatggaactctggaagcctgtcctctggcgtgcacacctttcccgccgtgctgcagtccgacctgtacacactgagctcctctgtgaccgtgcctagctcccccaggcctagcgagaccgtgacatgcaacgtggctcaccccgcctctagcacaaaggtggacaagaagatcgtgcctagagattgcggctgtaagccctgcatctgtaccgtgcctgaggtgagcagcgtgttcatctttccacccaagcctaaggacgtgctgaccatcacactgaccccaaaggtgacctgcgtggtggtggatatctctaaggacgatccagaggtgcagttcagctggtttgtggacgatgtggaggtgcacacagctcagacccagcctagggaggagcagttcaacagcaccttcaggagcgtgagcgagctgccaatcatgcaccaggattggctgaacggaaaggagttcaagtgccgggtgaactccgctgcctttccagcccccatcgagaagacaatctctaagaccaagggacagccacgggagccacaggtgtacaccctgcctccaagccgcgacgagctgacaaagaaccaggtgtccctgacctgtctggtgaagggattctacccaagcgatatcgctgtggagtgggagtccaacggccagcccgagaacaactacaagaccacaccccctgtgctggactccgatggatctttctttctgtacagcaagctgaccgtggacaagtcccgctggcagcagggcaacgtgttcagctgctccgtgatgcacgaggccctgcacaaccactacacacagaagtctctgagcctgtccccaggaaagaggggaaggaagaggagatctggcagcggagctaccaacttttccctgctgaagcaggccggagatgtggaggagaaccctggcccaatggtgctgcagacacaggtgttcatcagcctgctgctgtggatcagcggagcttacggctccatcgtgatgacacagacccccaagtttctgctggtgtccgccggcgacagggtgacaatcacctgtaaggcttcccagtctgtgagcaacgatgtggcctggtaccagcagaagcctggacagtccccaaagctgctgatctactacgcctctaaccggtacacaggcgtgcctgaccgcttcacaggctctggatacggcaccgacttcaccttcaccatcagcaccgtgcaggctgaggacctggccgtgtacttttgccagcaggattacttcagcccatacacatttggaggcggaaccaagctggagatcaagcggacagtggctgcccctagcgtgttcatcttcccacccagcgacgagcagctgaagtctggaaccgccagcgtggtgtgcctgctgaacaacttctacccacgcgaggctaaggtgcagtggaaggtggataacgccctgcagagcggcaactcccaggagtctgtgacagagcaggacagcaaggattccacctactctctgagctccacactgaccctgtccaaggctgactacgagaagcacaaggtgtacgcctgcgaggtgacacaccagggactgcggtctcccgtgaccaagagctttaaccgcggcgagtgt

SEQ ID NO: 98 - protein, 726 aa, Artificial Sequnce
  Chemically Synethsized
MDWTWRILFLVAAATGTHAEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARIYYGSPGAMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGSIVMTQTPKFLLVSAGDRVTITCKASQSVSNDVAWYQQKPGQSPKLLIYYASNRYTGVPDRFTGSGYGTDFTFTISTVQAEDLAVYFCQQDYFSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 99 - DNA, 360 bp, Artificial Sequnce
  Chemically Synethsized
gaggtcaaactgctggagagtgggggagggctggtgcagcctggcggctccctgaagctgtcttgcgcagcaagcggcttcgacttttctcggtactggatgagctgggtgagacaggcaccaggcaagggcctggagtggatcggcgagatcaaccccgatagctccaccatcaattacacaccttctctgaaggacaagttcatcatcagcagggataacgccaagaataccctgtatctgcagatgtccaaggtgaggtctgaggacacagccctgtactattgcgcccgcatctactatggcagcccaggcgccatggattactggggccagggcacctccgtgacagtgtctagc

SEQ ID NO: 100 - protein, 120 aa, Artificial Sequnce; identical to SEQ ID NO: 94
  Chemically Synethsized

SEQ ID NO: 101 - DNA, 324 bp, Artificial Sequnce
  Chemically Synethsized
tccatcgtgatgacccagacacccaagtttctgctggtgagcgccggcgacagagtgaccatcacctgtaaggccagccagtccgtgtctaacgatgtggcctggtatcagcagaagcctggccagagcccaaagctgctgatctactatgcctccaataggtacaccggagtgcctgaccgcttcaccggctccggctatggcacagatttcacctttacaatctctacagtgcaggccgaggacctggccgtgtacttttgccagcaggattacttctccccctataccttcggcggcggcacaaagctggagatcaagagg

SEQ ID NO: 102 - protein, 108 aa, Artificial Sequnce
  Chemically Synethsized
SIVMTQTPKFLLVSAGDRVTITCKASQSVSNDVAWYQQKPGQSPKLLIYYASNRYTGVPDRFTGSGYGTDFTFTISTVQAEDLAVYFCQQDYFSPYTFGGGTKLEIKR

SEQ ID NO: 103 - DNA, 2202 bp, Artificial Sequnce
  Chemically Synethsized
atggactggacttggagaatcctgttcctggtcgctgctgctaccggcacccacgctgaggtcaaactgctggagagtgggggagggctggtgcagcctggcggctccctgaagctgtcttgcgcagcaagcggcttcgacttttctcggtactggatgagctgggtgagacaggcaccaggcaagggcctggagtggatcggcgagatcaaccccgatagctccaccatcaattacacaccttctctgaaggacaagttcatcatcagcagggataacgccaagaataccctgtatctgcagatgtccaaggtgaggtctgaggacacagccctgtactattgcgcccgcatctactatggcagcccaggcgccatggattactggggccagggcacctccgtgacagtgtctagcgcctccaccaagggaccaagcgtgttcccactggcaccttcctctaagagcacctccggcggcacagccgccctgggctgtctggtgaaggactatttccctgagccagtgaccgtgagctggaactccggcgccctgaccagcggagtgcacacatttcctgccgtgctgcagagctccggcctgtactccctgtctagcgtggtgaccgtgccatcctctagcctgggcacccagacatatatctgcaacgtgaatcacaagccttctaatacaaaggtggacaagaaggtggagccaaagagctgtgataagacccacacatgccctccctgtccagcacctgagctgctgggcggcccaagcgtgttcctgtttccacccaagcccaaggacaccctgatgatctccagaaccccagaggtgacatgcgtggtggtggacgtgtctcacgaggaccccgaggtgaagttcaactggtacgtggatggcgtggaggtgcacaatgccaagaccaagccacgggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagccacgggagccacaggtgtacacactgcctccatctagagacgagctgaccaagaaccaggtgagcctgacatgtctggtgaagggcttttatccctccgatatcgccgtggagtgggagtctaatggccagcctgagaacaattacaagaccacaccccctgtgctggactctgatggcagcttctttctgtattccaagctgaccgtggacaagtctcggtggcagcagggcaacgtgttctcttgcagcgtgatgcacgaggccctgcacaatcactacacccagaagtccctgtctctgagccctggcaagaggggaaggaagcggagatccggctctggagccacaaactttagcctgctgaagcaggccggcgatgtggaggagaatcctggcccaatggtgctgcagacccaggtgttcatctccctgctgctgtggatcagcggcgcctacggctccatcgtgatgacccagacacccaagtttctgctggtgagcgccggcgacagagtgaccatcacctgtaaggccagccagtccgtgtctaacgatgtggcctggtatcagcagaagcctggccagagcccaaagctgctgatctactatgcctccaataggtacaccggagtgcctgaccgcttcaccggctccggctatggcacagatttcacctttacaatctctacagtgcaggccgaggacctggccgtgtacttttgccagcaggattacttctccccctataccttcggcggcggcacaaagctggagatcaagaggaccgtggccgcccctagcgtgttcatctttccaccctccgacgagcagctgaagagcggcacagcctccgtggtgtgcctgctgaacaatttctacccccgcgaggccaaggtgcagtggaaggtggataacgccctgcagtccggcaattctcaggagagcgtgaccgagcaggactccaaggattctacatatagcctgtcctctaccctgacactgagcaaggccgactacgagaagcacaaggtgtatgcctgcgaggtcactcaccaggggctgcgctcaccagtcacaaaatctttcaatagaggggaatgctgataa

SEQ ID NO: 104 - protein, 732 aa, Artificial Sequnce
  Chemically Synethsized
MDWTWRILFLVAAATGTHAEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARIYYGSPGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGSIVMTQTPKFLLVSAGDRVTITCKASQSVSNDVAWYQQKPGQSPKLLIYYASNRYTGVPDRFTGSGYGTDFTFTISTVQAEDLAVYFCQQDYFSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 105 - DNA, 15 bp, Artificial Sequnce
  Chemically Synethsized
gacacctatatgcac

SEQ ID NO: 106 - DNA, 51 bp, Artificial Sequnce
  Chemically Synethsized
aggattgatcctgcgaatggtaatactaaatatgacccgaagttccaggcc

SEQ ID NO: 107 - DNA, 30 bp, Artificial Sequnce
  Chemically Synethsized
tactacgactacgtgggagctatggactac

SEQ ID NO: 108 - protein, 5 aa, Artificial Sequnce
  Chemically Synethsized
DTYMH

SEQ ID NO: 109 - protein, 17 aa, Artificial Sequnce
  Chemically Synethsized
RIDPANGNTKYDPKFQA

SEQ ID NO: 110 - protein, 10 aa, Artificial Sequnce
  Chemically Synethsized
YYDYVGAMDY

SEQ ID NO: 111 - DNA, 414 bp, Artificial Sequnce
  Chemically Synethsized
atgaaatgcagctgggttatcttcttcctgatggcagtggttacaggggtcaattcagaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccaggccaaggccactataacagcaaacacatcctccaacacagcctacctgcacctcagcagcctgacatctgaggacactgccgtctattactgtgccccatactacgactacgtgggagctatggactactggggtcaaggaacctcagtcaccgtctcctca

SEQ ID NO: 112 - protein, 138 aa, Artificial Sequnce
  Chemically Synethsized
MKCSWVIFFLMAVVTGVNSEVQLQQSGAELVKPGASVKLSCTASGFNIKDTYMHWVKQRPEQGLEWIGRIDPANGNTKYDPKFQAKATITANTSSNTAYLHLSSLTSEDTAVYYCAPYYDYVGAMDYWGQGTSVTVSS

SEQ ID NO: 113 - DNA, 33 bp, Artificial Sequnce
  Chemically Synethsized
ctggcaagtcagaccattggtacatggttagca

SEQ ID NO: 114 - DNA, 21 bp, Artificial Sequnce
  Chemically Synethsized
gctgcaaccagcttggcagat

SEQ ID NO: 115 - DNA, 27 bp, Artificial Sequnce
  Chemically Synethsized
caacaactttacaatactcctctgacg

SEQ ID NO: 116 - protein, 11 aa, Artificial Sequnce
  Chemically Synethsized
LASQTIGTWLA

SEQ ID NO: 117 - protein, 7 aa, Artificial Sequnce
  Chemically Synethsized
AATSLAD

SEQ ID NO: 118 - protein, 9 aa, Artificial Sequnce
  Chemically Synethsized
QQLYNTPLT

SEQ ID NO: 119 - DNA, 381 bp, Artificial Sequnce
  Chemically Synethsized
atgaacatgctcactcagctcctgggattactgctgctctggtttgcaggtggtaaatgtgacattcagatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcaaccagcttggcagatggggtcccatcaaggttcagtggtagtggatctggcacaaaattttctttcaagatcagcagcctacaggctgaagattttgtaagttattactgtcaacaactttacaatactcctctgacgttcggtggaggcaccaagctggaaatcaaa

SEQ ID NO: 120 - protein, 127 aa, Artificial Sequnce
  Chemically Synethsized
MNMLTQLLGLLLLWFAGGKCDIQMTQSPASQSASLGESVTITCLASQTIGTWLAWYQQKPGKSPQLLIYAATSLADGVPSRFSGSGSGTKFSFKISSLQAEDFVSYYCQQLYNTPLTFGGGTKLEIK

SEQ ID NO: 121 - DNA, 357 bp, Artificial Sequnce
  Chemically Synethsized
gaggtgcagctgcagcagtccggagctgagctggtgaagccaggagccagcgtgaagctgtcctgcacagcctctggcttcaacatcaaggatacctacatgcactgggtgaagcagaggccagagcagggactggagtggatcggaagaatcgaccccgccaacggcaacaccaagtacgatcctaagttccaggctaaggccaccatcacagctaacacaagctccaacaccgcctacctgcacctgtctagcctgacaagcgaggacaccgccgtgtactactgtgccccttactacgactacgtgggcgctatggattactggggacagggcacatctgtgaccgtgtcctct

SEQ ID NO: 122 - protein, 119 aa, Artificial Sequnce
  Chemically Synethsized
EVQLQQSGAELVKPGASVKLSCTASGFNIKDTYMHWVKQRPEQGLEWIGRIDPANGNTKYDPKFQAKATITANTSSNTAYLHLSSLTSEDTAVYYCAPYYDYVGAMDYWGQGTSVTVSS

SEQ ID NO: 123 - DNA, 330 bp, Artificial Sequnce
  Chemically Synethsized
aagtgcgatatccagatgacccagtccccagcttctcagagcgcctccctgggagagtctgtgaccatcacatgtctggctagccagacaatcggcacctggctggcctggtaccagcagaagccaggaaagagcccccagctgctgatctacgctgccacaagcctggctgacggagtgccctcccggttttctggaagcggctccggaaccaagttctcctttaagatcagctccctgcaggccgaggatttcgtgtcttactactgccagcagctgtacaacacacccctgacctttggcggaggcaccaagctggagatcaagcgc

SEQ ID NO: 124 - protein, 110 aa, Artificial Sequnce
  Chemically Synethsized
KCDIQMTQSPASQSASLGESVTITCLASQTIGTWLAWYQQKPGKSPQLLIYAATSLADGVPSRFSGSGSGTKFSFKISSLQAEDFVSYYCQQLYNTPLTFGGGTKLEIKR

SEQ ID NO: 125 - DNA, 2214 bp, Artificial Sequnce
  Chemically Synethsized
atggactggacctggcgcatcctgttcctggtggctgctgctaccggaacacacgctgaggtgcagctgcagcagtccggagctgagctggtgaagccaggagccagcgtgaagctgtcctgcacagcctctggcttcaacatcaaggatacctacatgcactgggtgaagcagaggccagagcagggactggagtggatcggaagaatcgaccccgccaacggcaacaccaagtacgatcctaagttccaggctaaggccaccatcacagctaacacaagctccaacaccgcctacctgcacctgtctagcctgacaagcgaggacaccgccgtgtactactgtgccccttactacgactacgtgggcgctatggattactggggacagggcacatctgtgaccgtgtcctctgctaagaccacagctccaagcgtgtacccactggctcccgtgtgcggaggaaccacaggcagctccgtgaccctgggctgtctggtgaagggatacttcccagagcccgtgaccctgacatggaacagcggctccctgtctagcggagtgcacacatttcctgccctgctgcagtccggcctgtacaccctgtcctctagcgtgaccgtgacatctaacacatggccaagccagaccatcacatgcaacgtggctcaccccgcctcctctacaaaggtggacaagaagatcgagcctcgggtgccaatcacccagaacccatgtccccctctgaaggagtgcccaccctgtgctgcccctaacctgctgggcggaccaagcgtgttcatctttcctccaaagatcaaggatgtgctgatgatctctctgagccctatcgtgacctgcgtggtggtggacgtgtccgaggacgatccagatgtgcagatctcttggtttgtgaacaacgtggaggtgcacacagctcagacccagacacacagggaggactacaacagcaccctgagagtggtgtccgccctgccaatccagcaccaggactggatgtccggcaaggagttcaagtgcaaggtgaacaacaaggatctgcctgccccaatcgagcggacaatctctaagccaaagggacctgtgcgcgctccacaggtgtacgtgctgccacctccagctgaggagatgaccaagaaggagttctctctgacatgtatgatcaccggctttctgcctgctgagatcgccgtggattggacaagcaacggaagaaccgagcagaactacaagaacaccgctacagtgctggactccgatggctcttacttcatgtactccaagctgcgggtgcagaagtctacctgggagcgcggcagcctgtttgcctgttctgtggtgcacgaggtgctgcacaaccacctgaccacaaagacaatctcccggtctctgggcaagaggggaaggaagaggagaagcggcagcggcgctaccaacttctccctgctgaagcaggctggcgacgtggaggagaacccaggacctatggtgctgcagacacaggtgtttatctctctgctgctgtggatcagcggcgcctacggaaagtgcgatatccagatgacccagtccccagcttctcagagcgcctccctgggagagtctgtgaccatcacatgtctggctagccagacaatcggcacctggctggcctggtaccagcagaagccaggaaagagcccccagctgctgatctacgctgccacaagcctggctgacggagtgccctcccggttttctggaagcggctccggaaccaagttctcctttaagatcagctccctgcaggccgaggatttcgtgtcttactactgccagcagctgtacaacacacccctgacctttggcggaggcaccaagctggagatcaagcgcgctgacgctgcccctacagtgagcatcttccccccttctagcgagcagctgaccagcggaggagcttccgtggtgtgcttcctgaacaacttttaccccaaggacatcaacgtgaagtggaagatcgatggcagcgagaggcagaacggagtgctgaactcctggacagaccaggatagcaaggactccacctactctatgtcctctaccctgacactgaccaaggatgagtacgagaggcacaacagctacacatgcgaggccacccacaagacatctaccagccctatcgtgaagtccttcaacagaaacgagtgt

SEQ ID NO: 126 - protein, 738 aa, Artificial Sequnce
  Chemically Synethsized
MDWTWRILFLVAAATGTHAEVQLQQSGAELVKPGASVKLSCTASGFNIKDTYMHWVKQRPEQGLEWIGRIDPANGNTKYDPKFQAKATITANTSSNTAYLHLSSLTSEDTAVYYCAPYYDYVGAMDYWGQGTSVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGKRGRKRRSGSGATNFSLLKQAGDVEENPGPMVLQTQVFISLLLWISGAYGKCDIQMTQSPASQSASLGESVTITCLASQTIGTWLAWYQQKPGKSPQLLIYAATSLADGVPSRFSGSGSGTKFSFKISSLQAEDFVSYYCQQLYNTPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 127 - DNA, 357 bp, Artificial Sequnce
  Chemically Synethsized
gaagtgcagctgcagcagtctggagccgaactggtgaagccaggcgcctccgtgaagctgtcttgcacagccagcggcttcaacatcaaggacacctacatgcactgggtgaagcagaggcccgagcagggcctggagtggatcggaaggatcgacccagccaacggcaataccaagtacgatcccaagtttcaggccaaggccaccatcacagccaacacaagctccaataccgcctatctgcacctgtctagcctgacaagcgaggataccgccgtgtactattgtgccccttactatgactacgtgggcgccatggattattggggccagggcacatccgtgaccgtgtcctct

SEQ ID NO: 128 - protein, 119 aa, Artificial Sequnce; identical to SEQ ID NO: 122
  Chemically Synethsized

SEQ ID NO: 129 - DNA, 330 bp, Artificial Sequnce
  Chemically Synethsized
aagtgcgatatccagatgacccagagcccagcaagccagtccgcctctctgggagagtctgtgacaatcacctgtctggccagccagacaatcggcacctggctggcctggtaccagcagaagcctggcaagtccccacagctgctgatctatgcagccacatctctggcagacggagtgcctagccgcttcagcggctccggctctggaaccaagttctcctttaagatcagctccctgcaggccgaggatttcgtgtcttactattgccagcagctgtacaacacccctctgaccttcggcggcggcacaaagctggagatcaagagg

SEQ ID NO: 130 - protein, 110 aa, Artificial Sequnce; identical to SEQ ID NO: 124
  Chemically Synethsized

SEQ ID NO: 131 - DNA, 2193 bp, Artificial Sequnce
  Chemically Synethsized
atggattggacctggaggattctgtttctggtcgccgccgctactggaactcacgccgaagtgcagctgcagcagtctggagccgaactggtgaagccaggcgcctccgtgaagctgtcttgcacagccagcggcttcaacatcaaggacacctacatgcactgggtgaagcagaggcccgagcagggcctggagtggatcggaaggatcgacccagccaacggcaataccaagtacgatcccaagtttcaggccaaggccaccatcacagccaacacaagctccaataccgcctatctgcacctgtctagcctgacaagcgaggataccgccgtgtactattgtgccccttactatgactacgtgggcgccatggattattggggccagggcacatccgtgaccgtgtcctctgccagcaccaagggaccatccgtgttcccactggcaccttgctcccggtctacaagcgagtccaccgccgccctgggatgtctggtgaaggactacttccccgagcctgtgacagtgagctggaactccggcgccctgacaagcggagtgcacacctttccagccgtgctgcagagctccggcctgtactccctgtctagcgtggtgaccgtgccatcctctaattttggcacccagacatatacctgcaacgtggaccacaagcccagcaatacaaaggtggataagaccgtggagaggaagtgctgcgtggagtgccctccctgtccagccccacccgtggcaggcccttccgtgttcctgtttcctccaaagcctaaggacacactgatgatcagccgcacaccagaggtgacctgcgtggtggtggacgtgtcccacgaggaccccgaggtgcagttcaactggtacgtggatggcgtggaggtgcacaatgccaagaccaagcctcgggaggagcagttcaatagcaccttccgggtggtgagcgtgctgaccgtggtgcaccaggattggctgaacggcaaggagtataagtgcaaggtgtctaataagggcctgccagcccccatcgagaagacaatcagcaagaccaagggacagccacgggagccacaggtgtacaccctgcccccttccagagaggagatgacaaagaaccaggtgtctctgacctgtctggtgaagggcttctatcccagcgacatcgccgtggagtgggagtccaatggccagcctgagaacaattacaagaccacaccacccatgctggactctgatggcagcttctttctgtatagcaagctgaccgtggataagtccaggtggcagcagggcaacgtgttttcttgcagcgtgatgcacgaggccctgcacaatcactacacacagaagtccctgtctctgagcccaggcaagaggggaaggaagcggagatccggctctggagccaccaacttcagcctgctgaagcaggcaggcgacgtggaggagaatcctggaccaatggtgctgcagacacaggtgtttatctccctgctgctgtggatctctggcgcctatggcaagtgcgatatccagatgacccagagcccagcaagccagtccgcctctctgggagagtctgtgacaatcacctgtctggccagccagacaatcggcacctggctggcctggtaccagcagaagcctggcaagtccccacagctgctgatctatgcagccacatctctggcagacggagtgcctagccgcttcagcggctccggctctggaaccaagttctcctttaagatcagctccctgcaggccgaggatttcgtgtcttactattgccagcagctgtacaacacccctctgaccttcggcggcggcacaaagctggagatcaagaggaccgtggcagcaccatccgtgttcatctttcctccatctgacgagcagctgaagtccggcaccgcctctgtggtgtgcctgctgaacaacttctaccctagagaggccaaggtgcagtggaaggtggataacgccctgcagtccggcaattctcaggagagcgtgacagagcaggactccaaggattctacctatagcctgtctagcacactgaccctgagcaaggccgactacgagaagcacaaggtgtatgcctgtgaggtcacccaccaggggctgaggtcaccagtcaccaagtctttcaacaggggagaatgttgataa

SEQ ID NO: 132 - protein, 729 aa, Artificial Sequnce
  Chemically Synethsized
MDWTWRILFLVAAATGTHAEVQLQQSGAELVKPGASVKLSCTASGFNIKDTYMHWVKQRPEQGLEWIGRIDPANGNTKYDPKFQAKATITANTSSNTAYLHLSSLTSEDTAVYYCAPYYDYVGAMDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGKCDIQMTQSPASQSASLGESVTITCLASQTIGTWLAWYQQKPGKSPQLLIYAATSLADGVPSRFSGSGSGTKFSFKISSLQAEDFVSYYCQQLYNTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 133 - DNA, 3783 bp, Artificial Sequnce
  Chemically Synthesized
agccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 134 - protein, 1261 aa, Artificial Sequnce
  Chemically Synthesized
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 135 - DNA, 3843 bp, Artificial Sequnce
  Chemically Synthesized
atggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacataatga

SEQ ID NO: 136 - protein, 1279 aa, Artificial Sequnce
  Chemically Synthesized
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 137 - DNA, 3783 bp, Artificial Sequnce
  Chemically Synthesized
agccagtgtgtgaacctgaccaccagaacacagctgcctcctgcctacaccaacagcttcaccagaggagtctactacccagacaaggtgttcagaagctctgtgctgcacagcacccaggacctcttcctgcctttcttcagcaacgtgacctggttccacgccatccacgtgtctggcaccaacggcaccaagagatttgacaaccctgtgctgcctttcaatgatggtgtgtactttgccagcacagagaagagcaacatcatccgaggctggatctttggcaccaccctggacagcaaaacacagagcctgctgatcgtgaataatgccaccaacgtggtcatcaaggtgtgtgagttccagttctgcaatgaccctttcctgggcgtgtactaccacaagaacaacaagtcctggatggagtctgagttccgagtgtacagctctgccaacaactgcacatttgaatatgtgtcccagcctttcctgatggacctggagggcaagcagggcaatttcaagaacctgagagaatttgtgttcaagaacatcgatggctacttcaagatctacagcaagcacacacccatcaacctggtgagagatcttcctcagggcttctctgccctggagcctctggtggacctgcccatcggcatcaacatcacccgctttcagaccctgctggccctgcacagaagctacctgaccccaggagacagcagcagcggctggacagctggagctgctgcctactacgtgggctacctgcagccaagaaccttcctgctgaagtacaacgaaaatggcaccatcactgtggctgtggcctgtgccctggaccctctttctgagaccaagtgcaccctgaagtccttcacagtggagaaaggcatctaccagaccagcaacttcagagttcagccaacagagagcatcgtgagatttccaaacatcaccaacctgtgtccttttggagaagtcttcaatgccaccagatttgcttctgtgtacgcctggaacagaaaaagaatcagcaactgtgtggctgactactctgtgctgtacaactctgcctccttctccaccttcaagtgctacggtgtgtctcctaccaagctgaatgacctgtgcttcaccaacgtgtatgctgacagctttgtcatcagaggagatgaagtgcggcagatcgcccctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggagtggaaggcttcaactgctacttccctctgcagagctacggcttccagcccaccaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagttcctgcctttccagcagtttggaagagacattgctgacaccacagatgctgtgagagatcctcagaccctggagatcctggacatcacaccctgctcctttggaggagtttctgtcatcacacctggagccaacaccagcaaccaagtgacagtgctgtaccaagatgtgaactgcacagaagttcctgtggccatccacgctgaccagctgaccccaacctggagagtctacagcacaggcagcaacgtgtttaaaacaagagctggctgcctgattggagcagagcacgtgaacaacagctatgaatgtgacatccctattggagctggcatctgtgccagctaccagacccaaaccaacagcccaagaagagccaggagcacagccagccagagcatcatcgcctacaccatgagcctgggagcagagaactctgtggcctacagcaacaacagcatcgtcatccccaccaacttcaccatctctgtgaccacagagatcctgcctgtgtccatgaccaagacatctgtggactgcaccatgtacatctgcagtgacagcacagaatgcagcaaccctctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaacttctcccagatcctgcctgaccccagcaagcccagcaagagaagcttcattgaagacctgctgttcaacaaagtgaccctggctgatgctggcttcatcaaacaatatggagactgcctgggagacattgctgccagagacctgatctgtgcccagaagtttaatggcctgactgtgctgcctcctctgctgacagatgaaatgatcgcccagtacacatctgccctgctggctggcaccatcacatctggctggacatttggagctggagctgccctgcagatcccttttgccatgcagatggcctacagatttaatggcatcagagtgacccagaacgtgctgtatgaaaaccagaagctgatcgccaaccagttcaactctgccatcggcaagatccaggacagcctgagcagcacagcctctgccctgggcaagctgcaggatgtggtgaaccaaaatgcccaggccctgaacaccctggtgaagcagctgagcagcaccttctccaccatctccagcgtgctgaatgacatcctgagccggctggacaaggtggaagctgaggtgcagatcgacagactcatcacaggccggctgcagagcctgcagacctacgtgacccagcagctgatcagagctgctgagatcagagcttctgccaacctgaaggccaccaagatgtcagaatgtgtgctgggccagagcaagagagtggacttctgtggcaaaggctaccacctgatgtccttccctcagtctgctcctcacggcgtggtgttcctgcacgtgacctacgtgcctgcccaggagaagaacttcaccacagctcctgccacctgccacgatggcaaagcccacttcccaagagaaggcgtctttgtgtccaatggcacccactggttcgtgacccagagaaactttgatgagcctcagatcatcaccacagacaacacatttgtttctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcattgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 138 - protein, 1261 aa, Artificial Sequnce
  Chemically Synthesized
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITVAVACALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGANTSNQVTVLYQDVNCTEVPVAIHADQLTPTWRVYSTGSNVFKTRAGCLIGAEHVNNSYECDIPIGAGICASYQTQTNSPRRARSTASQSIIAYTMSLGAENSVAYSNNSIVIPTNFTISVTTEILPVSMTKTSVDCTMYICSDSTECSNPLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIRVTQNVLYENQKLIANQFNSAIGKIQDSLSSTASALGKLQDVVNQNAQALNTLVKQLSSTFSTISSVLNDILSRLDKVEAEVQIDRLITGRLQSLQTYVTQQLIRAAEIRASANLKATKMSECVLGQSKRVDFCGKGYHLMSFPQSAPHGVVFLHVTYVPAQEKNFTTAPATCHDGKAHFPREGVFVSNGTHWFVTQRNFDEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 139 - DNA, 3843 bp, Artificial Sequnce
  Chemically Synthesized
atggattggacctggattctttttctcgttgcagctgctacacgcgttcatagcagccagtgtgtgaacctgaccaccagaacacagctgcctcctgcctacaccaacagcttcaccagaggagtctactacccagacaaggtgttcagaagctctgtgctgcacagcacccaggacctcttcctgcctttcttcagcaacgtgacctggttccacgccatccacgtgtctggcaccaacggcaccaagagatttgacaaccctgtgctgcctttcaatgatggtgtgtactttgccagcacagagaagagcaacatcatccgaggctggatctttggcaccaccctggacagcaaaacacagagcctgctgatcgtgaataatgccaccaacgtggtcatcaaggtgtgtgagttccagttctgcaatgaccctttcctgggcgtgtactaccacaagaacaacaagtcctggatggagtctgagttccgagtgtacagctctgccaacaactgcacatttgaatatgtgtcccagcctttcctgatggacctggagggcaagcagggcaatttcaagaacctgagagaatttgtgttcaagaacatcgatggctacttcaagatctacagcaagcacacacccatcaacctggtgagagatcttcctcagggcttctctgccctggagcctctggtggacctgcccatcggcatcaacatcacccgctttcagaccctgctggccctgcacagaagctacctgaccccaggagacagcagcagcggctggacagctggagctgctgcctactacgtgggctacctgcagccaagaaccttcctgctgaagtacaacgaaaatggcaccatcactgtggctgtggcctgtgccctggaccctctttctgagaccaagtgcaccctgaagtccttcacagtggagaaaggcatctaccagaccagcaacttcagagttcagccaacagagagcatcgtgagatttccaaacatcaccaacctgtgtccttttggagaagtcttcaatgccaccagatttgcttctgtgtacgcctggaacagaaaaagaatcagcaactgtgtggctgactactctgtgctgtacaactctgcctccttctccaccttcaagtgctacggtgtgtctcctaccaagctgaatgacctgtgcttcaccaacgtgtatgctgacagctttgtcatcagaggagatgaagtgcggcagatcgcccctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggagtggaaggcttcaactgctacttccctctgcagagctacggcttccagcccaccaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagttcctgcctttccagcagtttggaagagacattgctgacaccacagatgctgtgagagatcctcagaccctggagatcctggacatcacaccctgctcctttggaggagtttctgtcatcacacctggagccaacaccagcaaccaagtgacagtgctgtaccaagatgtgaactgcacagaagttcctgtggccatccacgctgaccagctgaccccaacctggagagtctacagcacaggcagcaacgtgtttaaaacaagagctggctgcctgattggagcagagcacgtgaacaacagctatgaatgtgacatccctattggagctggcatctgtgccagctaccagacccaaaccaacagcccaagaagagccaggagcacagccagccagagcatcatcgcctacaccatgagcctgggagcagagaactctgtggcctacagcaacaacagcatcgtcatccccaccaacttcaccatctctgtgaccacagagatcctgcctgtgtccatgaccaagacatctgtggactgcaccatgtacatctgcagtgacagcacagaatgcagcaaccctctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaacttctcccagatcctgcctgaccccagcaagcccagcaagagaagcttcattgaagacctgctgttcaacaaagtgaccctggctgatgctggcttcatcaaacaatatggagactgcctgggagacattgctgccagagacctgatctgtgcccagaagtttaatggcctgactgtgctgcctcctctgctgacagatgaaatgatcgcccagtacacatctgccctgctggctggcaccatcacatctggctggacatttggagctggagctgccctgcagatcccttttgccatgcagatggcctacagatttaatggcatcagagtgacccagaacgtgctgtatgaaaaccagaagctgatcgccaaccagttcaactctgccatcggcaagatccaggacagcctgagcagcacagcctctgccctgggcaagctgcaggatgtggtgaaccaaaatgcccaggccctgaacaccctggtgaagcagctgagcagcaccttctccaccatctccagcgtgctgaatgacatcctgagccggctggacaaggtggaagctgaggtgcagatcgacagactcatcacaggccggctgcagagcctgcagacctacgtgacccagcagctgatcagagctgctgagatcagagcttctgccaacctgaaggccaccaagatgtcagaatgtgtgctgggccagagcaagagagtggacttctgtggcaaaggctaccacctgatgtccttccctcagtctgctcctcacggcgtggtgttcctgcacgtgacctacgtgcctgcccaggagaagaacttcaccacagctcctgccacctgccacgatggcaaagcccacttcccaagagaaggcgtctttgtgtccaatggcacccactggttcgtgacccagagaaactttgatgagcctcagatcatcaccacagacaacacatttgtttctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcattgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacataatga

SEQ ID NO: 140 - protein, 1279 aa, Artificial Sequnce
  Chemically Synthesized
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITVAVACALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGANTSNQVTVLYQDVNCTEVPVAIHADQLTPTWRVYSTGSNVFKTRAGCLIGAEHVNNSYECDIPIGAGICASYQTQTNSPRRARSTASQSIIAYTMSLGAENSVAYSNNSIVIPTNFTISVTTEILPVSMTKTSVDCTMYICSDSTECSNPLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIRVTQNVLYENQKLIANQFNSAIGKIQDSLSSTASALGKLQDVVNQNAQALNTLVKQLSSTFSTISSVLNDILSRLDKVEAEVQIDRLITGRLQSLQTYVTQQLIRAAEIRASANLKATKMSECVLGQSKRVDFCGKGYHLMSFPQSAPHGVVFLHVTYVPAQEKNFTTAPATCHDGKAHFPREGVFVSNGTHWFVTQRNFDEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 141 - protein, 18 aa, Artificial Sequnce
  Chemically Synthesized
MDWTWILFLVAAATRVHS

SEQ ID NO: 142 - protein, 9 aa, Artificial Sequnce
  Chemically Synthesized
PHGVVFLHV

SEQ ID NO: 143 - protein, 9 aa, Artificial Sequnce
  Chemically Synthesized
VVFLHVTYV

SEQ ID NO: 144 - protein, 15 aa, Artificial Sequnce
  Chemically Synthesized
VVLSFELLHAPATVC

SEQ ID NO: 145 - protein, 15 aa, Artificial Sequnce
  Chemically Synthesized
KNKCVNFNFNGLTGT

SEQ ID NO: 146 - protein, 15 aa, Artificial Sequnce
  Chemically Synthesized
LLHAPATVCGPKKST

SEQ ID NO: 147 - protein, 15 aa, Artificial Sequnce
  Chemically Synthesized
SFPQSAPHGVVFLHV

SEQ ID NO: 148 - protein, 15 aa, Artificial Sequnce
  Chemically Synthesized
FTTAPAICHDGKAHF

SEQ ID NO: 149 - protein, 15 aa, Artificial Sequnce
  Chemically Synthesized
PHGVVFLHVTYVPAQ

SEQ ID NO: 150 - protein, 15 aa, Artificial Sequnce
  Chemically Synthesized
YNYKLPDDFTGCVIA

SEQ ID NO: 151 - protein, 15 aa, Artificial Sequnce
  Chemically Synthesized
VGGNYNYLYRLFRKS

SEQ ID NO: 152 - protein, 15 aa, Artificial Sequnce
  Chemically Synthesized
DDFTGCVIAWNSNNL